(19) United States
(12) Patent Application Publication
(10) Pub. No.: US 2026/0201466 A1
(43) Pub. Date: Jul. 16, 2026

(54) SORTING METHOD USING BARCODED CHAMBERS FOR SINGLE CELL WORKFLOW

(71) Applicant: Becton, Dickinson and Company, Franklin Lakes, NJ (US)

(72) Inventors: Aruna Ayer, Santa Clara, CA (US); Rosary Kim Nguyen, San Jose, CA (US); Devon Joseph Jensen, Campbell, CA (US); Ricelle Agbayani Acob, Fremont, CA (US); Vadir Lopez-Salmeron, Heidelberg (DE)

(21) Appl. No.: 19/131,316

(22) PCT Filed: Dec. 18, 2023
(86) PCT No.: PCT/US2023/084669

§ 371 Date: May 20, 2025

Related U.S. Application Data
(60) Provisional application No. 63/387,997, filed on Dec. 19, 2022.
(60) Provisional application No. 63/607,516, filed on Dec. 7, 2023.

(51) Int. Cl. C12Q 1/6874; C12Q 1/6806

(52) U.S. Cl. CPC C12Q 1/6874; C12Q 1/6806

(57) ABSTRACT

Disclosed herein include systems, methods, compositions, and kits for methods of assigning the sequencing data of single cells to chambers. There are provided, in some embodiments, methods of associating sequencing data and phenotypic data of single cells. Disclosed herein include solid supports each comprising a plurality of oligonucleotide barcodes comprising a cell label sequence. Each cell label sequence can comprise predetermined chamber indexing subsequence(s). Oligonucleotide barcodes situated within the same chamber can comprise the same chamber indexing subsequence(s), and oligonucleotide barcodes situated within different chambers can comprise different chamber indexing subsequence(s).

### RELATED APPLICATIONS

The present application is a National Stage application of PCT Application No. PCT/US2023/084669, filed on Dec. 18, 2023, which claims priority to U.S. Provisional Application No. 63/387,997, filed on Dec. 19, 2022; and U.S. Provisional Application No. 63/607,516, filed on Dec. 7, 2023. The entire contents of these applications are hereby expressly incorporated by reference in their entireties.

### BACKGROUND

### Field

The present disclosure relates generally to the field of molecular biology, for example determining gene expression using molecular barcoding.

### Description of the Related Art

Current technology allows measurement of gene expression of single cells in a massively parallel manner (e.g., >10000 cells) by attaching cell specific oligonucleotide barcodes to poly(A) mRNA molecules from individual cells as each of the cells is co-localized with a barcoded reagent bead in a compartment. Phenotypic information regarding the single calls can be derived from experiments performed prior to the measurement of gene expression and/or protein expression via sequencing. There is a need for compositions, systems, and methods of associating sequencing data and phenotypic data of single cells. In order to correlate image or fluorescence-based sorting with single cell sequencing data, there is a need for compositions, systems, and methods of sorting cells into chambers such that the information is retained as to which population of cells is sorted into which chambers. Generally, there is a need for compositions, systems, and methods of assigning sequencing data to a chamber of a microwell array.

### SUMMARY

Disclosed herein include compositions. In some embodiments, the composition comprises two or more pluralities of solid supports. In some embodiments, the solid supports each comprise a plurality of oligonucleotide barcodes each comprising a cell label sequence, wherein each cell label sequence comprises predetermined chamber indexing subsequence(s). In some embodiments, oligonucleotide barcodes associated with the same solid support comprise the same cell label sequence, wherein oligonucleotide barcodes associated with different solid supports comprise different cell label sequences.

In some embodiments, the two or more pluralities of solid supports comprise a plurality of first solid supports and a plurality of second solid supports, wherein oligonucleotide barcodes associated with plurality of first solid supports have a first predetermined chamber indexing subsequence(s), wherein oligonucleotide barcodes associated with plurality of second solid supports have a second predetermined chamber indexing subsequence(s), and wherein the first predetermined chamber indexing subsequence(s) and the second predetermined chamber indexing subsequence(s) are different.

In some embodiments, the two or more pluralities of solid supports comprise a plurality of first solid supports and a plurality of second solid supports, wherein the first predetermined chamber indexing subsequence(s) of the plurality of first solid supports are selected from a first set of chamber indexing subsequence(s), wherein the second predetermined chamber indexing subsequence(s) of the plurality of second solid supports are selected from a second set of chamber indexing subsequence(s), and wherein each of the chamber indexing subsequence(s) of first set of chamber indexing subsequence(s) differ from the chamber indexing subsequence(s) of the second set of chamber indexing subsequence(s). In some embodiments, based on the predetermined chamber indexing subsequence(s) of a sequencing read derived from an oligonucleotide barcode, or a product thereof, a user can determine if the oligonucleotide barcode is associated with the plurality of first solid supports or the plurality of second solid supports.

The two or more pluralities of solid supports can comprise: a plurality of third solid supports, optionally: (a) oligonucleotide barcodes associated with the plurality of third solid supports have a third chamber indexing subsequence(s) and/or (b) third chamber indexing subsequence(s) of the plurality of third solid supports are selected from a third set of chamber indexing subsequence(s); a plurality of fourth solid supports, optionally: (a) oligonucleotide barcodes associated with the plurality of fourth solid supports have a fourth chamber indexing subsequence(s) and/or (b) fourth chamber indexing subsequence(s) of the plurality of fourth solid supports are selected from a fourth set of chamber indexing subsequence(s); a plurality of fifth solid supports, optionally: (a) oligonucleotide barcodes associated with the plurality of fifth solid supports have a fifth chamber indexing subsequence(s) and/or (b) fifth chamber indexing subsequence(s) of the plurality of fifth solid supports are selected from a fifth set of chamber indexing subsequence(s); a plurality of sixth solid supports, optionally: (a) oligonucleotide barcodes associated with the plurality of sixth solid supports have a sixth chamber indexing subsequence(s) and/or (b) sixth chamber indexing subsequence(s) of the plurality of sixth solid supports are selected from a sixth set of chamber indexing subsequence(s); a plurality of seventh solid supports, optionally: (a) oligonucleotide barcodes associated with the plurality of seventh solid supports have a seventh chamber indexing subsequence(s) and/or (b) seventh chamber indexing subsequence(s) of the plurality of seventh solid supports are selected from a seventh set of chamber indexing subsequence(s); and/or a plurality of eighth solid supports, optionally: (a) oligonucleotide barcodes associated with the plurality of eighth solid supports have a eighth chamber indexing subsequence(s) and/or (b) eighth chamber indexing subsequence(s) of the plurality of eighth solid supports are selected from an eighth set of chamber indexing subsequence(s). In some embodiments, the first chamber indexing subsequence(s), the second chamber indexing subsequence(s), the third chamber indexing subsequence(s), the fourth chamber indexing subsequence(s), the fifth chamber indexing subsequence(s), the sixth chamber indexing subsequence(s), the seventh chamber indexing subsequence(s), and/or the eighth chamber indexing subsequence(s), do not share a sequence with each other. In some embodiments, based on the predetermined chamber indexing subsequence(s) of a sequencing read derived from an oligonucleotide barcode, or a product thereof, a user can determine if the oligonucleotide barcode is associated with a first solid support, a second solid support, a third solid support, a fourth solid support, a fifth solid support, a sixth solid support, a seventh solid support, or an eighth solid support.

In some embodiments, the cell label comprises a plurality of cell label portions and one or more linkers. In some embodiments, the cell label comprises a first cell label portion, a first linker, and a second cell label portion, optionally the cell label comprises a second linker and a third cell label portion, further optionally the cell label comprises a third linker and a fourth cell label portion. In some embodiments, the predetermined chamber indexing subsequence(s) comprise the first cell label portion, the second cell label portion, the third cell label portion, the fourth cell label portion, or any combination thereof. In some embodiments, the first, second, third, fourth, fifth, sixth, seventh, and/or eighth set of chamber indexing subsequence(s) comprises a set of less than about 960, about 864, about 768, about 672, about 576, about 480, about 384, about 288, about 192, about 96, or about 48, unique sequences distinct from the chamber indexing subsequence(s) of other sets of chamber indexing subsequence(s). In some embodiments, the first cell label portion, the second cell label portion, the third cell label portion, the fourth cell label portion, or any combination thereof, are selected from a set of less than about 480, about 384, about 288, about 192, about 96, or about 48, unique sequences. In some embodiments, based on the sequence(s) of the first cell label portion, the second cell label portion, the third cell label portion, the fourth cell label portion, or any combination thereof, of a sequencing read derived from an oligonucleotide barcode, or a product thereof, a user can determine if the oligonucleotide barcode is associated with a first solid support, a second solid support, a third solid support, a fourth solid support, a fifth solid support, a sixth solid support, a seventh solid support, or an eighth solid support.

Disclosed herein include methods. In some embodiments, the method comprises: distributing a plurality of precursor first solid supports and a first population of first oligonucleotides to a first plurality of first partitions, wherein colocalized solid supports and first oligonucleotides become associated; distributing a plurality of precursor second solid supports and a second population of first oligonucleotides to a first plurality of second partitions, wherein colocalized solid supports and first oligonucleotides become associated; pooling the plurality of precursor first solid supports associated with first oligonucleotides; pooling the plurality of precursor second solid supports associated with first oligonucleotides; distributing the plurality of precursor first solid supports associated with first oligonucleotides and a first population of second oligonucleotides to a second plurality of first partitions, wherein colocalized solid supports and second oligonucleotides become associated; distributing the plurality of precursor second solid supports associated with first oligonucleotides and a second population of second oligonucleotides to a second plurality of second partitions, wherein colocalized solid supports and second oligonucleotides become associated; pooling the plurality of precursor first solid supports associated with first and second oligonucleotides; pooling the plurality of precursor second solid supports associated with first and second oligonucleotides; distributing the plurality of precursor first solid supports associated with first and second oligonucleotides and a first population of third oligonucleotides to a third plurality of first partitions, wherein colocalized solid supports and third oligonucleotides become associated; and distributing the plurality of precursor second solid supports associated with first and second oligonucleotides and a second population of third oligonucleotides to a third plurality of second partitions, wherein colocalized solid supports and third oligonucleotides become associated. In some embodiments, the method comprises: pooling the plurality of precursor first solid supports associated with first, second, and third oligonucleotides to generate a plurality of first solid supports; and pooling the plurality of precursor second solid supports associated with first, second, and third oligonucleotides to generate a plurality of second solid supports.

In some embodiments, (i) the first and second populations of first oligonucleotides are the same, the first and second populations of second oligonucleotides are the same, and the first and second populations of third oligonucleotides are different; (ii) the first and second populations of first oligonucleotides are the same, the first and second populations of second oligonucleotides are different, and the first and second populations of third oligonucleotides are the same; (iii) the first and second populations of first oligonucleotides are the same, the first and second populations of second oligonucleotides are different, and the first and second populations of third oligonucleotides are different; (iv) the first and second populations of first oligonucleotides are different, the first and second populations of second oligonucleotides are different, and the first and second populations of third oligonucleotides are different; (v) the first and second populations of first oligonucleotides are different, the first and second populations of second oligonucleotides are different, and the first and second populations of third oligonucleotides are the same; (vi) the first and second populations of first oligonucleotides are different, the first and second populations of second oligonucleotides are the same, and the first and second populations of third oligonucleotides are different; or (vii) the first and second populations of first oligonucleotides are different, the first and second populations of second oligonucleotides are the same, and the first and second populations of third oligonucleotides are the same.

The first and second populations of first oligonucleotides can each comprise about 192 first cell label portions with distinct sequences. In some embodiments, the first and second populations of second oligonucleotides each comprise about 192 second cell label portions with distinct sequences. In some embodiments, the first and second populations of third oligonucleotides each comprise about 192 third cell label portions with distinct sequences.

In some embodiments, the first and second populations of first oligonucleotides are the same; the first and second populations of second oligonucleotides are the same; and the first and second populations of third oligonucleotides are different. In some embodiments, the first and second populations of third oligonucleotides comprise non-overlapping subsets of the cell label portion sequences of the first and second populations of first oligonucleotides and/or the first and second populations of second oligonucleotides.

The first and second populations of first oligonucleotides can each comprise about 384 first cell label portions with distinct sequences. In some embodiments, the first and second populations of second oligonucleotides each comprise about 384 second cell label portions with distinct sequences. In some embodiments, the first and second populations of third oligonucleotides each comprise about 48 third cell label portions with distinct sequences.

In some embodiments, the method comprises: distributing the plurality of precursor first solid supports associated with first, second, and third oligonucleotides and fourth oligonucleotides to a fourth plurality of first partitions, wherein colocalized solid supports and fourth oligonucleotides become associated; distributing the plurality of precursor second solid supports associated with first, second, and third oligonucleotides and a fifth oligonucleotides to a fourth plurality of second partitions, wherein colocalized solid supports and fifth oligonucleotides become associated; pooling the plurality of precursor first solid supports associated with first, second, third, and fourth oligonucleotides to generate a plurality of first solid supports; and pooling the plurality of precursor second solid supports associated with first, second, third, and fifth oligonucleotides to generate a plurality of second solid supports.

In some embodiments, the first and second populations of first oligonucleotides are the same; the first and second populations of second oligonucleotides are the same; the first and second populations of third oligonucleotides are the same; and the fifth oligonucleotides and sixth oligonucleotides are different.

In some embodiments, the first oligonucleotide comprises a first cell label portion and a first linker, or complements thereof. In some embodiments, the second oligonucleotide comprises a first linker, a second cell label portion, and a second linker, or complements thereof. In some embodiments, the third oligonucleotide comprises a second linker and a third cell label portion, or complements thereof, optionally the second oligonucleotide further comprises a third linker or complement thereof. In some embodiments, the fourth oligonucleotide comprises a third linker and a fourth cell label portion, or complements thereof.

In some embodiments, the first oligonucleotide and the second oligonucleotide are configured to connect via the first linker. In some embodiments, the second oligonucleotide and the third oligonucleotide are configured to connect via the second linker. In some embodiments, the third oligonucleotide and the fourth oligonucleotide are configured to connect via the third linker. In some embodiments, the plurality of first solid supports and/or the plurality of second solid supports comprise at least about 1000, about 10000, about 100000, about 1000000, about 7000000, about 10000000, about 56000000, unique cell label sequences. In some embodiments, the sequence of the first second cell label portion, the second cell label portion, and/or the third cell label portion is the same. In some embodiments, the sequence of the first second cell label portion, the second cell label portion, and/or the third cell label portion is different. In some embodiments, the first oligonucleotide, the second oligonucleotide, the third oligonucleotide, and/or the fourth oligonucleotide are single-stranded, double-stranded, and/or comprise one or two single-stranded overhangs. In some embodiments, the first solid supports and second solid supports each comprise a plurality of oligonucleotide barcodes each comprising a cell label sequence, wherein each cell label sequence comprises predetermined chamber indexing subsequence(s).

In some embodiments, oligonucleotide barcodes associated with plurality of first solid supports have a first predetermined chamber indexing subsequence(s), oligonucleotide barcodes associated with plurality of second solid supports have a second predetermined chamber indexing subsequence(s), and the first predetermined chamber indexing subsequence(s) and the second predetermined chamber indexing subsequence(s) are different.

The first predetermined chamber indexing subsequence(s) of the plurality of first solid supports can be selected from a first set of chamber indexing subsequence(s), the second predetermined chamber indexing subsequence(s) of the plurality of second solid supports are selected from a second set of chamber indexing subsequence(s), and each of the chamber indexing subsequence(s) of first set of chamber indexing subsequence(s) differ from the chamber indexing subsequence(s) of the second set of chamber indexing subsequence(s). The methods provided herein can generate the compositions provided herein.

In some embodiments, the method comprises: providing a microwell array comprising a plurality of chambers, wherein each chamber comprises a plurality of partitions; partitioning the plurality of first solid supports and the plurality of second solid supports to the partitions of an identified first chamber and identified second chamber of the plurality of chambers, respectively. In some embodiments, distributing oligonucleotides to a plurality of partitions comprises providing a plurality of partitions comprising said oligonucleotides. In some embodiments, the plurality of partitions comprise a 384-well plate, a 288-well plate, a 192-well plate, a 96-well plate or a 48-well plate.

In some embodiments, the solid support comprises a synthetic particle. In some embodiments, at least one oligonucleotide barcode of the plurality of oligonucleotide barcodes is immobilized on the synthetic particle, partially immobilized on the synthetic particle, enclosed in the synthetic particle, partially enclosed in the synthetic particle, or a combination thereof. In some embodiments, the synthetic particle is disruptable. In some embodiments, the synthetic particle comprises a bead. In some embodiments, the bead comprises a Sepharose bead, a streptavidin bead, an agarose bead, a magnetic bead, a conjugated bead, a protein A conjugated bead, a protein G conjugated bead, a protein A/G conjugated bead, a protein L conjugated bead, an oligo(dT) conjugated bead, a silica bead, a silica-like bead, an anti-biotin microbead, an anti-fluorochrome microbead, or any combination thereof. In some embodiments, the synthetic particle comprises a material selected from the group consisting of polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, and any combination thereof. In some embodiments, the synthetic particle comprises a disruptable hydrogel particle.

Disclosed herein include methods of assigning sequencing data to chambers. In some embodiments, the method comprises: providing a microwell array comprising a plurality of chambers, wherein each chamber comprises a plurality of partitions; partitioning each of two or more pluralities of solid supports to the partitions of an identified chamber of the plurality of chambers, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s); partitioning each of two or more populations of single cells to the partitions of an identified chamber of the plurality of chambers, wherein each of the two or more populations of single cells comprises a plurality of single cells, wherein the single cells comprise copies of a nucleic acid target, wherein single cells of the same population are situated within the same chamber, and wherein single cells of different populations are situated within different chambers; barcoding the copies of a nucleic acid target from at least one of the plurality of single cells of at least one population of single cells using the plurality of oligonucleotide barcodes to generate a plurality of barcoded nucleic acid targets; obtaining sequencing data comprising a plurality of sequencing reads of the plurality of barcoded nucleic acid targets, or products thereof; identifying the chamber indexing subsequence(s) of each cell label sequence in the sequencing data; and assigning each of the plurality of sequencing reads to a chamber of the plurality of chambers based on the chamber indexing subsequence(s) of each cell label sequence in the sequencing data.

Disclosed herein include methods of assigning sequencing data to chambers. In some embodiments, the method comprises: providing a microwell array comprising a plurality of chambers, wherein each chamber comprises a plurality of partitions, wherein each chamber comprises a predetermined plurality of solid supports selected from two or more pluralities of solid supports, wherein the solid supports are situated within the partitions of said chambers, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s); partitioning each of two or more populations of single cells to the partitions of an identified chamber of the plurality of chambers, wherein each of the two or more populations of single cells comprises a plurality of single cells, wherein the single cells comprise copies of a nucleic acid target, wherein single cells of the same population are situated within the same chamber, and wherein single cells of different populations are situated within different chambers; barcoding the copies of a nucleic acid target from at least one of the plurality of single cells of at least one population of single cells using the plurality of oligonucleotide barcodes to generate a plurality of barcoded nucleic acid targets; obtaining sequencing data comprising a plurality of sequencing reads of the plurality of barcoded nucleic acid targets, or products thereof; identifying the chamber indexing subsequence(s) of each cell label sequence in the sequencing data; and assigning each of the plurality of sequencing reads to a chamber of the plurality of chambers based on the chamber indexing subsequence(s) of each cell label sequence in the sequencing data.

Disclosed herein include methods of assigning sequencing data to a population of single cells. In some embodiments, the method comprises: providing a microwell array comprising a plurality of chambers, wherein each chamber comprises a plurality of partitions; partitioning each of two or more pluralities of solid supports to the partitions of an identified chamber of the plurality of chambers, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s); partitioning each of two or more populations of single cells to the partitions of an identified chamber of the plurality of chambers, wherein each of the two or more populations of single cells comprises a plurality of single cells, wherein the single cells comprise copies of a nucleic acid target, wherein single cells of the same population are situated within the same chamber, and wherein single cells of different populations are situated within different chambers; barcoding the copies of a nucleic acid target from at least one of the plurality of single cells of at least one population of single cells using the plurality of oligonucleotide barcodes to generate a plurality of barcoded nucleic acid targets; obtaining sequencing data comprising a plurality of sequencing reads of the plurality of barcoded nucleic acid targets, or products thereof; identifying the chamber indexing subsequence(s) of each cell label sequence in the sequencing data; assigning each of the plurality of sequencing reads to a chamber of the plurality of chambers based on the chamber indexing subsequence(s) of each cell label sequence in the sequencing data; and assigning each of the plurality of sequencing reads to a population of the two or more populations of single cells based on the chamber assigned to the sequencing read.

Disclosed herein include methods of assigning sequencing data to a population of single cells. In some embodiments, the method comprises: providing a microwell array comprising a plurality of chambers, wherein each chamber comprises a plurality of partitions, wherein each chamber comprises a predetermined plurality of solid supports selected from two or more pluralities of solid supports, wherein the solid supports are situated within the partitions of said chambers, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s); partitioning each of two or more populations of single cells to the partitions of an identified chamber of the plurality of chambers, wherein each of the two or more populations of single cells comprises a plurality of single cells, wherein the single cells comprise copies of a nucleic acid target, wherein single cells of the same population are situated within the same chamber, and wherein single cells of different populations are situated within different chambers; barcoding the copies of a nucleic acid target from at least one of the plurality of single cells of at least one population of single cells using the plurality of oligonucleotide barcodes to generate a plurality of barcoded nucleic acid targets; obtaining sequencing data comprising a plurality of sequencing reads of the plurality of barcoded nucleic acid targets, or products thereof; identifying the chamber indexing subsequence(s) of each cell label sequence in the sequencing data; and assigning each of the plurality of sequencing reads to a chamber of the plurality of chambers based on the chamber indexing subsequence(s) of each cell label sequence in the sequencing data; and assigning each of the plurality of sequencing reads to a population of the two or more populations of single cells based on the chamber assigned to the sequencing read.

Disclosed herein include methods of associating sequencing data and phenotypic data of a population of single cells. In some embodiments, the method comprises: obtaining phenotypic data of each of two or more populations of single cells; providing a microwell array comprising a plurality of chambers, wherein each chamber comprises a plurality of partitions; partitioning each of two or more pluralities of solid supports to the partitions of an identified chamber of the plurality of chambers, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s); partitioning each of two or more populations of single cells to the partitions of an identified chamber of the plurality of chambers, wherein each of the two or more populations of single cells comprises a plurality of single cells, wherein the single cells comprise copies of a nucleic acid target, wherein single cells of the same population are situated within the same chamber, and wherein single cells of different populations are situated within different chambers; barcoding the copies of a nucleic acid target from at least one of the plurality of single cells of at least one population of single cells using the plurality of oligonucleotide barcodes to generate a plurality of barcoded nucleic acid targets; obtaining sequencing data comprising a plurality of sequencing reads of the plurality of barcoded nucleic acid targets, or products thereof; identifying the chamber indexing subsequence(s) of each cell label sequence in the sequencing data; and associating the sequencing data and the phenotypic data of at least one population of single cells based on the chamber indexing subsequence(s) of each cell label sequence in the sequencing data.

Disclosed herein include methods of associating sequencing data and phenotypic data of a population of single cells. In some embodiments, the method comprises: obtaining phenotypic data of each of two or more populations of single cells; providing a microwell array comprising a plurality of chambers, wherein each chamber comprises a plurality of partitions, wherein each chamber comprises a predetermined plurality of solid supports selected from two or more pluralities of solid supports, wherein the solid supports are situated within the partitions of said chambers, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s); partitioning each of two or more populations of single cells to the partitions of an identified chamber of the plurality of chambers, wherein each of the two or more populations of single cells comprises a plurality of single cells, wherein the single cells comprise copies of a nucleic acid target, wherein single cells of the same population are situated within the same chamber, and wherein single cells of different populations are situated within different chambers; barcoding the copies of a nucleic acid target from at least one of the plurality of single cells of at least one population of single cells using the plurality of oligonucleotide barcodes to generate a plurality of barcoded nucleic acid targets; obtaining sequencing data comprising a plurality of sequencing reads of the plurality of barcoded nucleic acid targets, or products thereof; identifying the chamber indexing subsequence(s) of each cell label sequence in the sequencing data; and associating the sequencing data and the phenotypic data of at least one population of single cells based on the chamber indexing subsequence(s) of each cell label sequence in the sequencing data.

The method can comprise: assigning each of the plurality of sequencing reads to a population of the two or more populations of single cells based on the chamber assigned to the sequencing read. The method can comprise: obtaining phenotypic data of each of two or more populations of single cells; and associating the sequencing data and the phenotypic data of at least one population of single cells based on the chamber indexing subsequence(s) of each cell label sequence in the sequencing data.

In some embodiments, the two or more pluralities of solid supports are the two or more pluralities of solid supports of the compositions provided herein and/or are generated by the methods provided herein. In some embodiments, the two or more pluralities of solid supports comprise a plurality of first solid supports and a plurality of second solid supports. In some embodiments, oligonucleotide barcodes associated with plurality of first solid supports have a first predetermined chamber indexing subsequence(s). In some embodiments, oligonucleotide barcodes associated with plurality of second solid supports have a second predetermined chamber indexing subsequence(s). In some embodiments, the first predetermined chamber indexing subsequence(s) and the second predetermined chamber indexing subsequence(s) are different.

In some embodiments, the two or more pluralities of solid supports comprise a plurality of first solid supports and a plurality of second solid supports. In some embodiments, the first predetermined chamber indexing subsequence(s) of the plurality of first solid supports are selected from a first set of chamber indexing subsequence(s). In some embodiments, the second predetermined chamber indexing subsequence(s) of the plurality of second solid supports are selected from a second set of chamber indexing subsequence(s). In some embodiments, each of the chamber indexing subsequence(s) of first set of chamber indexing subsequence(s) differ from the chamber indexing subsequence(s) of the second set of chamber indexing subsequence(s). In some embodiments, based on the predetermined chamber indexing subsequence(s) of a sequencing read derived from an oligonucleotide barcode, or a product thereof, a user can determine if the oligonucleotide barcode is associated with the plurality of first solid supports or the plurality of second solid supports.

Partitioning each of two or more pluralities of solid supports to the partitions of an identified chamber of the plurality of chambers can comprise partitioning a predetermined plurality of solid supports selected from two or more pluralities of solid supports to an identified chamber of the plurality of chambers. In some embodiments, barcoding the copies of a nucleic acid target comprises: contacting the plurality of oligonucleotide barcodes with the copies of a nucleic acid target for hybridization; and extending the plurality of oligonucleotide barcodes hybridized to the copies of a nucleic acid target to generate a plurality of barcoded nucleic acid targets,

The partition can be a microwell having a volume ranging from about 1,000 μm³ to about 786,000 μm³. In some embodiments, the oligonucleotide barcodes each comprise a molecular label sequence. In some embodiments, oligonucleotide barcodes of the same plurality of solid supports comprise the same chamber indexing subsequence(s), and wherein oligonucleotide barcodes of different pluralities of solid supports comprise different chamber indexing subsequence(s). In some embodiments, each cell label of the plurality of oligonucleotide barcodes comprises at least 6 nucleotides. In some embodiments, the cell label comprises a plurality of portions and one or more linkers. In some embodiments, the cell label comprises a first cell label portion, a first linker, and a second cell label portion, optionally the cell label comprises a second linker and a third cell label portion, further optionally the cell label comprises a third linker and a fourth cell label portion. In some embodiments, the first cell label portion comprises the chamber indexing subsequence(s). The chamber indexing subsequence(s) can be 2-15 nucleotides in length.

In some embodiments, the populations of single cells are distinct samples. In some embodiments, each population of the two or more populations of single cells is a biological replicate sample, a technical replicate sample, a control sample, an experimental sample, or a combination thereof. In some embodiments, the two or more populations of single cells are derived from one or more samples separated on the basis of phenotypic data, optionally the plurality of single cells comprises T cells, B cells, tumor cells, myeloid cells, blood cells, normal cells, fetal cells, maternal cells, or a mixture thereof. In some embodiments, the plurality of chambers is at least about 4 chambers, about 10 chambers, about 20 chambers, about 40 chambers, about 60 chambers, about 80 chambers, about 100 chambers, about 200 chambers, about 300 chambers, about 400 chambers, about 500 chambers, about 750 chambers, or about 1000 chambers. In some embodiments, the plurality of partitions is at least about 100 partitions, about 500 partitions, about 1000 partitions, about 5000 partitions, about 10000 partitions, about 25000 partitions, about 50000 partitions, about 75000 partitions, or about 100000 partitions. In some embodiments, the sequencing of the cell label identifies the chamber of origin of each sequenced barcoded nucleic acid target, or products thereof, within the microwell array.

In some embodiments, a partition of the plurality of partitions comprises a single cell of the plurality of single cells and a single solid support of the plurality of solid supports. In some embodiments, the plurality of barcoded nucleic acid targets each comprise a sequence complementary to at least a portion of the nucleic acid target and the molecular label. In some embodiments, each of the plurality of sequencing reads comprise (1) a cell label sequence and (2) a molecular label sequence. In some embodiments, each oligonucleotide barcode comprises a first universal sequence. In some embodiments, the oligonucleotide barcode comprises a target-binding region comprising a capture sequence. In some embodiments, the target-binding region comprises a gene-specific sequence, an oligo(dT) sequence, a random multimer, or any combination thereof. In some embodiments, partitioning a plurality of solid supports to the partitions of each of the plurality of chambers comprises flow cytometrically depositing each plurality of solid supports into an identified chamber of the microwell array. In some embodiments, partitioning each of two or more populations of single cells to the partitions of an identified chamber of the plurality of chambers comprises flow cytometrically depositing each population of single cells into an identified chamber of the microwell array. The method can comprise: aligning a sorting component of a flow cytometer with the microwell array.

The phenotypic data can comprise event data. The event data can comprise quantitative biological event data derived from a sorting device. In some embodiments, the event data comprises a side scatter signal, a forward scatter signal, one or more fluorescence signals, or any combination thereof. The method can comprise: correlation analysis of the phenotypic data and the sequencing data of the single cells. In some embodiments, the correlation analysis identifies one or more of the following: candidate biomarkers, candidate therapeutic agents, candidate doses of therapeutic agents, and/or cellular targets of candidate therapeutic agents. The method can comprise: lysing the one or more of the single cells. The viability of the single cells can be maintained for a period of time after partitioning and before lysis. The period of time can be at least about 10 min, 20 min, 30 min, 40 min, 50 min, 60 min, 100 min, 250 min, 500 min, 750 min, 1000 min, 2500 min, 5000 min, 7500 min, or 10000 min.

The method can comprise: determining the copy number of the nucleic acid target in one or more of the plurality of single cells of at least one population of single cells. In some embodiments, determining the copy number of the nucleic acid target in one or more of the plurality of single cells comprises determining the copy number of the nucleic acid target in the plurality of single cells based on the number of molecular labels with distinct sequences, complements thereof, or a combination thereof, associated with the plurality of barcoded nucleic acid targets, or products thereof.

The method can comprise: contacting random primers with the plurality of barcoded nucleic acid targets, wherein each of the random primers comprises a second universal sequence, or a complement thereof; and extending the random primers hybridized to the plurality of barcoded nucleic acid targets to generate a plurality of extension products. The method can comprise: amplifying the plurality of extension products using primers capable of hybridizing to the first universal sequence or complements thereof, and primers capable of hybridizing the second universal sequence or complements thereof, thereby generating a first plurality of barcoded amplicons. In some embodiments, amplifying the plurality of extension products comprises adding sequences of binding sites of sequencing primers and/or sequencing adaptors, complementary sequences thereof, and/or portions thereof, to the plurality of extension products. The method can comprise: determining the copy number of the nucleic acid target in one or more of the plurality of single cells based on the number of molecular labels with distinct sequences associated with the first plurality of barcoded amplicons, or products thereof. In some embodiments, determining the copy number of the nucleic acid target in one or more of the plurality of single cells comprises determining the number of each of the plurality of nucleic acid targets in one or more of the plurality of single cells based on the number of the molecular labels with distinct sequences associated with barcoded amplicons of the first plurality of barcoded amplicons comprising a sequence of the each of the plurality of nucleic acid targets. In some embodiments, the sequence of the each of the plurality of nucleic acid targets comprises a subsequence of the each of the plurality of nucleic acid targets. In some embodiments, the sequence of the nucleic acid target in the first plurality of barcoded amplicons comprises a subsequence of the nucleic acid target. The method can comprise: amplifying the first plurality of barcoded amplicons using primers capable of hybridizing to the first universal sequence or complements thereof, and primers capable of hybridizing the second universal sequence or complements thereof, thereby generating a second plurality of barcoded amplicons. In some embodiments, amplifying the first plurality of barcoded amplicons comprises adding sequences of binding sites of sequencing primers and/or sequencing adaptors, complementary sequences thereof, and/or portions thereof, to the first plurality of barcoded amplicons. The method can comprise: determining the copy number of the nucleic acid target in one or more of the plurality of single cells based on the number of molecular labels with distinct sequences associated with the second plurality of barcoded amplicons, or products thereof. In some embodiments, the first plurality of barcoded amplicons and/or the second plurality of barcoded amplicons comprise whole transcriptome amplification (WTA) products.

The method can comprise: synthesizing a third plurality of barcoded amplicons using the plurality of barcoded nucleic acid targets as templates to generate a third plurality of barcoded amplicons. In some embodiments, synthesizing a third plurality of barcoded amplicons comprises performing polymerase chain reaction (PCR) amplification of the plurality of the barcoded nucleic acid targets. In some embodiments, synthesizing a third plurality of barcoded amplicons comprises PCR amplification using primers capable of hybridizing to the first universal sequence, or a complement thereof, and a target-specific primer. The method can comprise: obtaining sequence data of the third plurality of barcoded amplicons, or products thereof, and optionally obtaining the sequence information comprises attaching sequencing adaptors to the third plurality of barcoded amplicons, or products thereof. The method can comprise: determining the copy number of the nucleic acid target in one or more of the plurality of single cells based on the number of molecular labels with distinct sequences associated with the third plurality of barcoded amplicons, or products thereof.

In some embodiments, the nucleic acid target comprises a nucleic acid molecule. In some embodiments, the nucleic acid molecule comprises ribonucleic acid (RNA), messenger RNA (mRNA), microRNA, small interfering RNA (siRNA), RNA degradation product, RNA comprising a poly(A) tail, or any combination thereof. In some embodiments, the nucleic acid target comprises a sample indexing oligonucleotide, and optionally the sample indexing oligonucleotide comprises a sample indexing sequence, and sample indexing sequences of at least two sample indexing compositions of a plurality of sample indexing compositions comprise different sequences. In some embodiments, the nucleic acid target comprises a cellular component-binding reagent specific oligonucleotide. In some embodiments, a cellular component-binding reagent specific oligonucleotide comprises a unique identifier sequence for a cellular component-binding reagent. In some embodiments, extending the plurality of oligonucleotide barcodes comprises extending the plurality of oligonucleotide barcodes using a reverse transcriptase and/or a DNA polymerase lacking at least one of 5′ to 3′ exonuclease activity and 3′ to 5′ exonuclease activity. In some embodiments, the DNA polymerase comprises a Klenow Fragment. In some embodiments, the reverse transcriptase comprises a viral reverse transcriptase, optionally wherein the viral reverse transcriptase is a murine leukemia virus (MLV) reverse transcriptase or a Moloney murine leukemia virus (MMLV) reverse transcriptase. In some embodiments, the first universal sequence and the second universal sequence are the same. In some embodiments, the first universal sequence and the second universal sequence are different. In some embodiments, the first universal sequence, and/or the second universal sequence comprise the binding sites of sequencing primers and/or sequencing adaptors, complementary sequences thereof, and/or portions thereof. In some embodiments, the sequencing adaptors comprise a P5 sequence, a P7 sequence, complementary sequences thereof, and/or portions thereof. In some embodiments, the sequencing primers comprise a Read 1 sequencing primer, a Read 2 sequencing primer, complementary sequences thereof, and/or portions thereof. In some embodiments, at least 10 of the plurality of oligonucleotide barcodes comprise different molecular label sequences. In some embodiments, each molecular label of the plurality of oligonucleotide barcodes comprises at least 6 nucleotides.

In some embodiments, the solid support comprises a synthetic particle. In some embodiments, the solid support comprises a planar surface. In some embodiments, at least one of the plurality of oligonucleotide barcodes is immobilized on, partially immobilized, enclosed in, or partially enclosed in the synthetic particle. In some embodiments, the synthetic particle is disruptable. In some embodiments, the synthetic particle comprises a bead. In some embodiments, the bead comprises a Sepharose bead, a streptavidin bead, an agarose bead, a magnetic bead, a conjugated bead, a protein A conjugated bead, a protein G conjugated bead, a protein A/G conjugated bead, a protein L conjugated bead, an oligo(dT) conjugated bead, a silica bead, a silica-like bead, an anti-biotin microbead, an anti-fluorochrome microbead, or any combination thereof. In some embodiments, the synthetic particle comprises a material selected from polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, and a combination thereof. The synthetic particle can be a disruptable hydrogel particle.

Disclosed herein include compositions. In some embodiments, the composition comprises: a microwell array, wherein the microwell array comprises a plurality of chambers, wherein each chamber comprises a plurality of partitions, wherein each partition is a microwell having a volume ranging from about 1,000 μm³ to about 786,000 μm³. The composition can comprise: a cartridge, wherein the cartridge comprises at least one of: an inlet port, an outlet port, a pump, a valve, a vent, a reservoir, a sample collection chamber, a temperature control apparatus, or any combination thereof.

Disclosed herein include compositions. In some embodiments, the composition comprises: a cartridge, wherein the cartridge comprises at least one of: an inlet port, an outlet port, a pump, a valve, a vent, a reservoir, a sample collection chamber, a temperature control apparatus, or any combination thereof, wherein the cartridge comprises a microwell array, wherein the microwell array comprises a plurality of chambers, wherein each chamber comprises a plurality of partitions, wherein each partition is a microwell having a volume ranging from about 1,000 μm³ to about 786,000 μm³.

Disclosed herein include compositions. In some embodiments, the composition comprises: two or more pluralities of solid supports, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes of the same plurality of solid supports comprise the same chamber indexing subsequence(s), and wherein oligonucleotide barcodes of different pluralities of solid supports comprise different chamber indexing subsequence(s). In some embodiments, the two or more pluralities of solid supports are the two or more pluralities of solid supports of the compositions disclosed herein and/or are generated by a method disclosed herein.

In some embodiments, a partition of the plurality of partitions comprises a single solid support of a plurality of solid supports, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s).

The composition can comprise: a chamber indexing subsequence(s) lookup table. The chamber indexing subsequence(s) lookup table can, for example, identify the chamber indexing subsequence(s) associated with each solid support distributed in each microwell of the array.

In some embodiments, the cartridge is configured to maintain the viability of single cells partitioned within said microwells, optionally the period of time is at least about 10 min, 20 min, 30 min, 40 min, 50 min, 60 min, 100 min, 250 min, 500 min, 750 min, 1000 min, 2500 min, 5000 min, 7500 min, or 10000 min. In some embodiments, the cartridge comprises a transparent window for optical imaging of the microwells. The composition can comprise: an imaging system configured to capture and process images of all or a portion of the microwells, wherein the imaging system further comprises an illumination subsystem, an imaging subsystem, and a processor. In some embodiments, the imaging system is configured to perform bright-field, dark-field, fluorescence, or quantitative phase imaging. The composition can comprise: a buffer. The composition can comprise: one or more reagents for a reverse transcription reaction, one or more reagents for an amplification reaction, or both.

In some embodiments, the oligonucleotide barcodes each comprise a molecular label sequence. In some embodiments, each cell label of the plurality of oligonucleotide barcodes comprises at least 6 nucleotides. In some embodiments, the cell label comprises a plurality of portions and one or more linkers. In some embodiments, the cell label comprises a first cell label portion, a first linker, and a second cell label portion, optionally the cell label comprises a second linker and a third cell label portion, further optionally the cell label comprises a third linker and a fourth cell label portion. In some embodiments, the first cell label portion comprises the chamber indexing subsequence(s). In some embodiments, the chamber indexing subsequence(s) is 2-15 nucleotides in length. In some embodiments, the plurality of chambers is at least about 4 chambers, about 10 chambers, about 20 chambers, about 40 chambers, about 60 chambers, about 80 chambers, about 100 chambers, about 200 chambers, about 300 chambers, about 400 chambers, about 500 chambers, about 750 chambers, or about 1000 chambers. In some embodiments, the plurality of partitions is at least about 100 partitions, about 500 partitions, about 1000 partitions, about 5000 partitions, about 10000 partitions, about 25000 partitions, about 50000 partitions, about 75000 partitions, or about 100000 partitions.

The composition can comprise: a plurality of solid supports each comprising a plurality of oligonucleotide barcodes. In some embodiments, the oligonucleotide barcodes each comprise a molecular label and a cell label. In some embodiments, oligonucleotide barcodes associated with the same solid support comprise the same cell label sequence, and wherein oligonucleotide barcodes associated with different solid supports comprise different cell label sequences. In some embodiments, each oligonucleotide barcode comprises a first universal sequence. In some embodiments, the oligonucleotide barcode comprises a target-binding region comprising a capture sequence. In some embodiments, the target-binding region comprises a gene-specific sequence, an oligo(dT) sequence, a random multimer, or any combination thereof. In some embodiments, each molecular label of the plurality of oligonucleotide barcodes comprises at least 6 nucleotides.

In some embodiments, the solid support comprises a planar surface. In some embodiments, the solid support comprises a synthetic particle. In some embodiments, at least one oligonucleotide barcode of the plurality of oligonucleotide barcodes is immobilized on the synthetic particle, partially immobilized on the synthetic particle, enclosed in the synthetic particle, partially enclosed in the synthetic particle, or a combination thereof. In some embodiments, the synthetic particle is disruptable. In some embodiments, the synthetic particle comprises a bead. In some embodiments, the bead comprises a Sepharose bead, a streptavidin bead, an agarose bead, a magnetic bead, a conjugated bead, a protein A conjugated bead, a protein G conjugated bead, a protein A/G conjugated bead, a protein L conjugated bead, an oligo(dT) conjugated bead, a silica bead, a silica-like bead, an anti-biotin microbead, an anti-fluorochrome microbead, or any combination thereof. In some embodiments, the synthetic particle comprises a material selected from polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, and a combination thereof. The synthetic particle can be a disruptable hydrogel particle. In some embodiments, the composition further comprises instructions for use.

### BRIEF DESCRIPTION OF THE DRAWINGS

FIG. 1 illustrates a non-limiting exemplary barcode.

FIG. 2 shows a non-limiting exemplary workflow of barcoding and digital counting.

FIG. 3 is a schematic illustration showing a non-limiting exemplary process for generating an indexed library of targets barcoded at the 3′-ends from a plurality of targets.

FIGS. 4A-4B depict non-limiting exemplary schematics of a microwell array (FIG. 4A) and chamber (FIG. 4B) provided herein.

FIG. 5 depicts a non-limiting exemplary method for manufacturing oligonucleotide barcodes comprising chamber indexing subsequence(s) provided herein.

FIG. 6 depicts a non-limiting exemplary method for manufacturing oligonucleotide barcodes comprising chamber indexing subsequence(s) provided herein.

FIG. 7 depicts a non-limiting exemplary method for manufacturing oligonucleotide barcodes comprising chamber indexing subsequence(s) provided herein.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Quantifying small numbers of nucleic acids, for example messenger ribonucleotide acid (mRNA) molecules, is clinically important for determining, for example, the genes that are expressed in a cell at different stages of development or under different environmental conditions. However, it can also be very challenging to determine the absolute number of nucleic acid molecules (e.g., mRNA molecules), especially when the number of molecules is very small. One method to determine the absolute number of molecules in a sample is digital polymerase chain reaction (PCR). Ideally, PCR produces an identical copy of a molecule at each cycle. However, PCR can have disadvantages such that each molecule replicates with a stochastic probability, and this probability varies by PCR cycle and gene sequence, resulting in amplification bias and inaccurate gene expression measurements. Stochastic barcodes with unique molecular labels (also referred to as molecular indexes (MIs)) can be used to count the number of molecules and correct for amplification bias. Stochastic barcoding, such as the Precise™ assay (Cellular Research, Inc. (Palo Alto, CA)) and Rhapsody™ assay (Becton, Dickinson and Company (Franklin Lakes, NJ)), can correct for bias induced by PCR and library preparation steps by using molecular labels (MLS) to label mRNAs during reverse transcription (RT).

The Precise™ assay can utilize a non-depleting pool of stochastic barcodes with large number, for example 6561 to 65536, unique molecular label sequences on poly(T) oligonucleotides to hybridize to all poly(A)-mRNAs in a sample during the RT step. A stochastic barcode can comprise a universal PCR priming site. During RT, target gene molecules react randomly with stochastic barcodes. Each target molecule can hybridize to a stochastic barcode resulting to generate stochastically barcoded complementary ribonucleotide acid (cDNA) molecules). After labeling, stochastically barcoded cDNA molecules from microwells of a microwell plate can be pooled into a single tube for PCR amplification and sequencing. Raw sequencing data can be analyzed to produce the number of reads, the number of stochastic barcodes with unique molecular label sequences, and the numbers of mRNA molecules.

Disclosed herein include compositions. In some embodiments, the composition comprises two or more pluralities of solid supports. In some embodiments, the solid supports each comprise a plurality of oligonucleotide barcodes each comprising a cell label sequence, wherein each cell label sequence comprises predetermined chamber indexing subsequence(s). In some embodiments, oligonucleotide barcodes associated with the same solid support comprise the same cell label sequence, wherein oligonucleotide barcodes associated with different solid supports comprise different cell label sequences.

Disclosed herein include methods. In some embodiments, the method comprises: distributing a plurality of precursor first solid supports and a first population of first oligonucleotides to a first plurality of first partitions, wherein colocalized solid supports and first oligonucleotides become associated; distributing a plurality of precursor second solid supports and a second population of first oligonucleotides to a first plurality of second partitions, wherein colocalized solid supports and first oligonucleotides become associated; pooling the plurality of precursor first solid supports associated with first oligonucleotides; pooling the plurality of precursor second solid supports associated with first oligonucleotides; distributing the plurality of precursor first solid supports associated with first oligonucleotides and a first population of second oligonucleotides to a second plurality of first partitions, wherein colocalized solid supports and second oligonucleotides become associated; distributing the plurality of precursor second solid supports associated with first oligonucleotides and a second population of second oligonucleotides to a second plurality of second partitions, wherein colocalized solid supports and second oligonucleotides become associated; pooling the plurality of precursor first solid supports associated with first and second oligonucleotides; pooling the plurality of precursor second solid supports associated with first and second oligonucleotides; distributing the plurality of precursor first solid supports associated with first and second oligonucleotides and a first population of third oligonucleotides to a third plurality of first partitions, wherein colocalized solid supports and third oligonucleotides become associated; and distributing the plurality of precursor second solid supports associated with first and second oligonucleotides and a second population of third oligonucleotides to a third plurality of second partitions, wherein colocalized solid supports and third oligonucleotides become associated.

Disclosed herein include methods of assigning sequencing data to chambers. In some embodiments, the method comprises: providing a microwell array comprising a plurality of chambers, wherein each chamber comprises a plurality of partitions; partitioning each of two or more pluralities of solid supports to the partitions of an identified chamber of the plurality of chambers, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s); partitioning each of two or more populations of single cells to the partitions of an identified chamber of the plurality of chambers, wherein each of the two or more populations of single cells comprises a plurality of single cells, wherein the single cells comprise copies of a nucleic acid target, wherein single cells of the same population are situated within the same chamber, and wherein single cells of different populations are situated within different chambers; barcoding the copies of a nucleic acid target from at least one of the plurality of single cells of at least one population of single cells using the plurality of oligonucleotide barcodes to generate a plurality of barcoded nucleic acid targets; obtaining sequencing data comprising a plurality of sequencing reads of the plurality of barcoded nucleic acid targets, or products thereof; identifying the chamber indexing subsequence(s) of each cell label sequence in the sequencing data; and assigning each of the plurality of sequencing reads to a chamber of the plurality of chambers based on the chamber indexing subsequence(s) of each cell label sequence in the sequencing data.

Disclosed herein include methods of assigning sequencing data to chambers. In some embodiments, the method comprises: providing a microwell array comprising a plurality of chambers, wherein each chamber comprises a plurality of partitions, wherein each chamber comprises a predetermined plurality of solid supports selected from two or more pluralities of solid supports, wherein the solid supports are situated within the partitions of said chambers, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s); partitioning each of two or more populations of single cells to the partitions of an identified chamber of the plurality of chambers, wherein each of the two or more populations of single cells comprises a plurality of single cells, wherein the single cells comprise copies of a nucleic acid target, wherein single cells of the same population are situated within the same chamber, and wherein single cells of different populations are situated within different chambers; barcoding the copies of a nucleic acid target from at least one of the plurality of single cells of at least one population of single cells using the plurality of oligonucleotide barcodes to generate a plurality of barcoded nucleic acid targets; obtaining sequencing data comprising a plurality of sequencing reads of the plurality of barcoded nucleic acid targets, or products thereof; identifying the chamber indexing subsequence(s) of each cell label sequence in the sequencing data; and assigning each of the plurality of sequencing reads to a chamber of the plurality of chambers based on the chamber indexing subsequence(s) of each cell label sequence in the sequencing data.

Disclosed herein include methods of assigning sequencing data to a population of single cells. In some embodiments, the method comprises: providing a microwell array comprising a plurality of chambers, wherein each chamber comprises a plurality of partitions; partitioning each of two or more pluralities of solid supports to the partitions of an identified chamber of the plurality of chambers, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s); partitioning each of two or more populations of single cells to the partitions of an identified chamber of the plurality of chambers, wherein each of the two or more populations of single cells comprises a plurality of single cells, wherein the single cells comprise copies of a nucleic acid target, wherein single cells of the same population are situated within the same chamber, and wherein single cells of different populations are situated within different chambers; barcoding the copies of a nucleic acid target from at least one of the plurality of single cells of at least one population of single cells using the plurality of oligonucleotide barcodes to generate a plurality of barcoded nucleic acid targets; obtaining sequencing data comprising a plurality of sequencing reads of the plurality of barcoded nucleic acid targets, or products thereof; identifying the chamber indexing subsequence(s) of each cell label sequence in the sequencing data; assigning each of the plurality of sequencing reads to a chamber of the plurality of chambers based on the chamber indexing subsequence(s) of each cell label sequence in the sequencing data; and assigning each of the plurality of sequencing reads to a population of the two or more populations of single cells based on the chamber assigned to the sequencing read.

Disclosed herein include methods of assigning sequencing data to a population of single cells. In some embodiments, the method comprises: providing a microwell array comprising a plurality of chambers, wherein each chamber comprises a plurality of partitions, wherein each chamber comprises a predetermined plurality of solid supports selected from two or more pluralities of solid supports, wherein the solid supports are situated within the partitions of said chambers, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s); partitioning each of two or more populations of single cells to the partitions of an identified chamber of the plurality of chambers, wherein each of the two or more populations of single cells comprises a plurality of single cells, wherein the single cells comprise copies of a nucleic acid target, wherein single cells of the same population are situated within the same chamber, and wherein single cells of different populations are situated within different chambers; barcoding the copies of a nucleic acid target from at least one of the plurality of single cells of at least one population of single cells using the plurality of oligonucleotide barcodes to generate a plurality of barcoded nucleic acid targets; obtaining sequencing data comprising a plurality of sequencing reads of the plurality of barcoded nucleic acid targets, or products thereof; identifying the chamber indexing subsequence(s) of each cell label sequence in the sequencing data; and assigning each of the plurality of sequencing reads to a chamber of the plurality of chambers based on the chamber indexing subsequence(s) of each cell label sequence in the sequencing data; and assigning each of the plurality of sequencing reads to a population of the two or more populations of single cells based on the chamber assigned to the sequencing read.

Disclosed herein include methods of associating sequencing data and phenotypic data of a population of single cells. In some embodiments, the method comprises: obtaining phenotypic data of each of two or more populations of single cells; providing a microwell array comprising a plurality of chambers, wherein each chamber comprises a plurality of partitions; partitioning each of two or more pluralities of solid supports to the partitions of an identified chamber of the plurality of chambers, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s); partitioning each of two or more populations of single cells to the partitions of an identified chamber of the plurality of chambers, wherein each of the two or more populations of single cells comprises a plurality of single cells, wherein the single cells comprise copies of a nucleic acid target, wherein single cells of the same population are situated within the same chamber, and wherein single cells of different populations are situated within different chambers; barcoding the copies of a nucleic acid target from at least one of the plurality of single cells of at least one population of single cells using the plurality of oligonucleotide barcodes to generate a plurality of barcoded nucleic acid targets; obtaining sequencing data comprising a plurality of sequencing reads of the plurality of barcoded nucleic acid targets, or products thereof; identifying the chamber indexing subsequence(s) of each cell label sequence in the sequencing data; and associating the sequencing data and the phenotypic data of at least one population of single cells based on the chamber indexing subsequence(s) of each cell label sequence in the sequencing data.

Disclosed herein include methods of associating sequencing data and phenotypic data of a population of single cells. In some embodiments, the method comprises: obtaining phenotypic data of each of two or more populations of single cells; providing a microwell array comprising a plurality of chambers, wherein each chamber comprises a plurality of partitions, wherein each chamber comprises a predetermined plurality of solid supports selected from two or more pluralities of solid supports, wherein the solid supports are situated within the partitions of said chambers, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s); partitioning each of two or more populations of single cells to the partitions of an identified chamber of the plurality of chambers, wherein each of the two or more populations of single cells comprises a plurality of single cells, wherein the single cells comprise copies of a nucleic acid target, wherein single cells of the same population are situated within the same chamber, and wherein single cells of different populations are situated within different chambers; barcoding the copies of a nucleic acid target from at least one of the plurality of single cells of at least one population of single cells using the plurality of oligonucleotide barcodes to generate a plurality of barcoded nucleic acid targets; obtaining sequencing data comprising a plurality of sequencing reads of the plurality of barcoded nucleic acid targets, or products thereof; identifying the chamber indexing subsequence(s) of each cell label sequence in the sequencing data; and associating the sequencing data and the phenotypic data of at least one population of single cells based on the chamber indexing subsequence(s) of each cell label sequence in the sequencing data.

The method can comprise: assigning each of the plurality of sequencing reads to a population of the two or more populations of single cells based on the chamber assigned to the sequencing read. The method can comprise: obtaining phenotypic data of each of two or more populations of single cells; and associating the sequencing data and the phenotypic data of at least one population of single cells based on the chamber indexing subsequence(s) of each cell label sequence in the sequencing data.

Disclosed herein include compositions. In some embodiments, the composition comprises: a microwell array, wherein the microwell array comprises a plurality of chambers, wherein each chamber comprises a plurality of partitions, wherein each partition is a microwell having a volume ranging from about 1,000 μm³ to about 786,000 μm³. The composition can comprise: a cartridge, wherein the cartridge comprises at least one of: an inlet port, an outlet port, a pump, a valve, a vent, a reservoir, a sample collection chamber, a temperature control apparatus, or any combination thereof.

Disclosed herein include compositions. In some embodiments, the composition comprises: a cartridge, wherein the cartridge comprises at least one of: an inlet port, an outlet port, a pump, a valve, a vent, a reservoir, a sample collection chamber, a temperature control apparatus, or any combination thereof, wherein the cartridge comprises a microwell array, wherein the microwell array comprises a plurality of chambers, wherein each chamber comprises a plurality of partitions, wherein each partition is a microwell having a volume ranging from about 1,000 μm³ to about 786,000 μm³.

Disclosed herein include compositions. In some embodiments, the composition comprises: two or more pluralities of solid supports, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes of the same plurality of solid supports comprise the same chamber indexing subsequence(s), and wherein oligonucleotide barcodes of different pluralities of solid supports comprise different chamber indexing subsequence(s).

### Definitions

Unless defined otherwise, technical and scientific terms used herein have the same meaning as commonly understood by one of ordinary skill in the art to which the present disclosure belongs. See, e.g., Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “adaptor” can mean a sequence to facilitate amplification or sequencing of associated nucleic acids. The associated nucleic acids can comprise target nucleic acids. The associated nucleic acids can comprise one or more of spatial labels, target labels, sample labels, indexing label, or barcode sequences (e.g., molecular labels). The adaptors can be linear. The adaptors can be pre-adenylated adaptors. The adaptors can be double- or single-stranded. One or more adaptor can be located on the 5′ or 3′ end of a nucleic acid. When the adaptors comprise known sequences on the 5′ and 3′ ends, the known sequences can be the same or different sequences. An adaptor located on the 5′ and/or 3′ ends of a polynucleotide can be capable of hybridizing to one or more oligonucleotides immobilized on a surface. An adaptor can, in some embodiments, comprise a universal sequence. A universal sequence can be a region of nucleotide sequence that is common to two or more nucleic acid molecules. The two or more nucleic acid molecules can also have regions of different sequence. Thus, for example, the 5′ adaptors can comprise identical and/or universal nucleic acid sequences and the 3′ adaptors can comprise identical and/or universal sequences. A universal sequence that may be present in different members of a plurality of nucleic acid molecules can allow the replication or amplification of multiple different sequences using a single universal primer that is complementary to the universal sequence. Similarly, at least one, two (e.g., a pair) or more universal sequences that may be present in different members of a collection of nucleic acid molecules can allow the replication or amplification of multiple different sequences using at least one, two (e.g., a pair) or more single universal primers that are complementary to the universal sequences. Thus, a universal primer includes a sequence that can hybridize to such a universal sequence. The target nucleic acid sequence-bearing molecules may be modified to attach universal adaptors (e.g., non-target nucleic acid sequences) to one or both ends of the different target nucleic acid sequences. The one or more universal primers attached to the target nucleic acid can provide sites for hybridization of universal primers. The one or more universal primers attached to the target nucleic acid can be the same or different from each other.

As used herein the term “associated” or “associated with” can mean that two or more species are identifiable as being co-located at a point in time. An association can mean that two or more species are or were within a similar container. An association can be an informatics association. For example, digital information regarding two or more species can be stored and can be used to determine that one or more of the species were co-located at a point in time. An association can also be a physical association. In some embodiments, two or more associated species are “tethered”, “attached”, or “immobilized” to one another or to a common solid or semisolid surface. An association may refer to covalent or non-covalent means for attaching labels to solid or semi-solid supports such as beads. An association may be a covalent bond between a target and a label. An association can comprise hybridization between two molecules (such as a target molecule and a label).

As used herein, the term “complementary” can refer to the capacity for precise pairing between two nucleotides. For example, if a nucleotide at a given position of a nucleic acid is capable of hydrogen bonding with a nucleotide of another nucleic acid, then the two nucleic acids are considered to be complementary to one another at that position. Complementarity between two single-stranded nucleic acid molecules may be “partial,” in which only some of the nucleotides bind, or it may be complete when total complementarity exists between the single-stranded molecules. A first nucleotide sequence can be said to be the “complement” of a second sequence if the first nucleotide sequence is complementary to the second nucleotide sequence. A first nucleotide sequence can be said to be the “reverse complement” of a second sequence, if the first nucleotide sequence is complementary to a sequence that is the reverse (i.e., the order of the nucleotides is reversed) of the second sequence. As used herein, a “complementary” sequence can refer to a “complement” or a “reverse complement” of a sequence. It is understood from the disclosure that if a molecule can hybridize to another molecule it may be complementary, or partially complementary, to the molecule that is hybridizing.

As used herein, the term “digital counting” can refer to a method for estimating a number of target molecules in a sample. Digital counting can include the step of determining a number of unique labels that have been associated with targets in a sample. This methodology, which can be stochastic in nature, transforms the problem of counting molecules from one of locating and identifying identical molecules to a series of yes/no digital questions regarding detection of a set of predefined labels.

As used herein, the term “label” or “labels” can refer to nucleic acid codes associated with a target within a sample. A label can be, for example, a nucleic acid label. A label can be an entirely or partially amplifiable label. A label can be entirely or partially sequenceable label. A label can be a portion of a native nucleic acid that is identifiable as distinct. A label can be a known sequence. A label can comprise a junction of nucleic acid sequences, for example a junction of a native and non-native sequence. As used herein, the term “label” can be used interchangeably with the terms, “index”, “tag,” or “label-tag.” Labels can convey information. For example, in various embodiments, labels can be used to determine an identity of a sample, a source of a sample, an identity of a cell, and/or a target.

As used herein, the term “non-depleting reservoirs” can refer to a pool of barcodes (e.g., stochastic barcodes) made up of many different labels. A non-depleting reservoir can comprise large numbers of different barcodes such that when the non-depleting reservoir is associated with a pool of targets each target is likely to be associated with a unique barcode. The uniqueness of each labeled target molecule can be determined by the statistics of random choice, and depends on the number of copies of identical target molecules in the collection compared to the diversity of labels. The size of the resulting set of labeled target molecules can be determined by the stochastic nature of the barcoding process, and analysis of the number of barcodes detected then allows calculation of the number of target molecules present in the original collection or sample. When the ratio of the number of copies of a target molecule present to the number of unique barcodes is low, the labeled target molecules are highly unique (i.e., there is a very low probability that more than one target molecule will have been labeled with a given label).

As used herein, the term “nucleic acid” refers to a polynucleotide sequence, or fragment thereof. A nucleic acid can comprise nucleotides. A nucleic acid can be exogenous or endogenous to a cell. A nucleic acid can exist in a cell-free environment. A nucleic acid can be a gene or fragment thereof. A nucleic acid can be DNA. A nucleic acid can be RNA. A nucleic acid can comprise one or more analogs (e.g., altered backbone, sugar, or nucleobase). Some non-limiting examples of analogs include: 5-bromouracil, peptide nucleic acid, xeno nucleic acid, morpholinos, locked nucleic acids, glycol nucleic acids, threose nucleic acids, dideoxynucleotides, cordycepin, 7-deaza-GTP, fluorophores (e.g., rhodamine or fluorescein linked to the sugar), thiol containing nucleotides, biotin linked nucleotides, fluorescent base analogs, CpG islands, methyl-7-guanosine, methylated nucleotides, inosine, thiouridine, pseudouridine, dihydrouridine, queuosine, and wyosine. “Nucleic acid”, “polynucleotide, “target polynucleotide”, and “target nucleic acid” can be used interchangeably.

A nucleic acid can comprise one or more modifications (e.g., a base modification, a backbone modification), to provide the nucleic acid with a new or enhanced feature (e.g., improved stability). A nucleic acid can comprise a nucleic acid affinity tag. A nucleoside can be a base-sugar combination. The base portion of the nucleoside can be a heterocyclic base. The two most common classes of such heterocyclic bases are the purines and the pyrimidines. Nucleotides can be nucleosides that further include a phosphate group covalently linked to the sugar portion of the nucleoside. For those nucleosides that include a pentofuranosyl sugar, the phosphate group can be linked to the 2′, the 3′, or the 5′ hydroxyl moiety of the sugar. In forming nucleic acids, the phosphate groups can covalently link adjacent nucleosides to one another to form a linear polymeric compound. In turn, the respective ends of this linear polymeric compound can be further joined to form a circular compound; however, linear compounds are generally suitable. In addition, linear compounds may have internal nucleotide base complementarity and may therefore fold in a manner as to produce a fully or partially double-stranded compound. Within nucleic acids, the phosphate groups can commonly be referred to as forming the internucleoside backbone of the nucleic acid. The linkage or backbone can be a 3′ to 5′ phosphodiester linkage.

A nucleic acid can comprise a modified backbone and/or modified internucleoside linkages. Modified backbones can include those that retain a phosphorus atom in the backbone and those that do not have a phosphorus atom in the backbone. Suitable modified nucleic acid backbones containing a phosphorus atom therein can include, for example, phosphorothioates, chiral phosphorothioates, phosphorodithioates, phosphotriesters, aminoalkyl phosphotriesters, methyl and other alkyl phosphonate such as 3′-alkylene phosphonates, 5′-alkylene phosphonates, chiral phosphonates, phosphinates, phosphoramidates including 3′-amino phosphoramidate and aminoalkyl phosphoramidates, phosphorodiamidates, thionophosphoramidates, thionoalkylphosphonates, thionoalkylphosphotriesters, selenophosphates, and boranophosphates having normal 3′-5′ linkages, 2′-5′ linked analogs, and those having inverted polarity wherein one or more internucleotide linkages is a 3′ to 3′, a 5′ to 5′ or a 2′ to 2′ linkage.

A nucleic acid can comprise polynucleotide backbones that are formed by short chain alkyl or cycloalkyl internucleoside linkages, mixed heteroatom and alkyl or cycloalkyl internucleoside linkages, or one or more short chain heteroatomic or heterocyclic internucleoside linkages. These can include those having morpholino linkages (formed in part from the sugar portion of a nucleoside); siloxane backbones; sulfide, sulfoxide and sulfone backbones; formacetyl and thioformacetyl backbones; methylene formacetyl and thioformacetyl backbones; riboacetyl backbones; alkene containing backbones; sulfamate backbones; methyleneimino and methylenehydrazino backbones; sulfonate and sulfonamide backbones; amide backbones; and others having mixed N, O, S and CH₂ component parts.

A nucleic acid can comprise a nucleic acid mimetic. The term “mimetic” can be intended to include polynucleotides wherein only the furanose ring or both the furanose ring and the internucleotide linkage are replaced with non-furanose groups, replacement of only the furanose ring can also be referred as being a sugar surrogate. The heterocyclic base moiety or a modified heterocyclic base moiety can be maintained for hybridization with an appropriate target nucleic acid. One such nucleic acid can be a peptide nucleic acid (PNA). In a PNA, the sugar-backbone of a polynucleotide can be replaced with an amide containing backbone, in particular an aminoethylglycine backbone. The nucleotides can be retained and are bound directly or indirectly to aza nitrogen atoms of the amide portion of the backbone. The backbone in PNA compounds can comprise two or more linked aminoethylglycine units which gives PNA an amide containing backbone. The heterocyclic base moieties can be bound directly or indirectly to aza nitrogen atoms of the amide portion of the backbone.

A nucleic acid can comprise a morpholino backbone structure. For example, a nucleic acid can comprise a 6-membered morpholino ring in place of a ribose ring. In some of these embodiments, a phosphorodiamidate or other non-phosphodiester internucleoside linkage can replace a phosphodiester linkage.

A nucleic acid can comprise linked morpholino units (e.g., morpholino nucleic acid) having heterocyclic bases attached to the morpholino ring. Linking groups can link the morpholino monomeric units in a morpholino nucleic acid. Non-ionic morpholino-based oligomeric compounds can have less undesired interactions with cellular proteins. Morpholino-based polynucleotides can be nonionic mimics of nucleic acids. A variety of compounds within the morpholino class can be joined using different linking groups. A further class of polynucleotide mimetic can be referred to as cyclohexenyl nucleic acids (CeNA). The furanose ring normally present in a nucleic acid molecule can be replaced with a cyclohexenyl ring. CeNA DMT protected phosphoramidite monomers can be prepared and used for oligomeric compound synthesis using phosphoramidite chemistry. The incorporation of CeNA monomers into a nucleic acid chain can increase the stability of a DNA/RNA hybrid. CeNA oligoadenylates can form complexes with nucleic acid complements with similar stability to the native complexes. A further modification can include Locked Nucleic Acids (LNAs) in which the 2′-hydroxyl group is linked to the 4′ carbon atom of the sugar ring thereby forming a 2′-C, 4′-C-oxymethylene linkage thereby forming a bicyclic sugar moiety. The linkage can be a methylene (—CH₂), group bridging the 2′ oxygen atom and the 4′ carbon atom wherein n is 1 or 2. LNA and LNA analogs can display very high duplex thermal stabilities with complementary nucleic acid (Tm=+3 to +10° C.), stability towards 3′-exonucleolytic degradation and good solubility properties.

A nucleic acid may also include nucleobase (often referred to simply as “base”) modifications or substitutions. As used herein, “unmodified” or “natural” nucleobases can include the purine bases, (e.g., adenine (A) and guanine (G)), and the pyrimidine bases, (e.g., thymine (T), cytosine (C) and uracil (U)). Modified nucleobases can include other synthetic and natural nucleobases such as 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl (—C═C—CH₃) uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-aminoadenine, 8-azaguanine and 8-azaadenine, 7-deazaguanine and 7-deazaadenine and 3-deazaguanine and 3-deazaadenine. Modified nucleobases can include tricyclic pyrimidines such as phenoxazine cytidine (1H-pyrimido(5,4-b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b)(1,4)benzoxazin-2(3H)-one), phenothiazine cytidine (1H-pyrimido(5,4-b)(1,4)benzothiazin-2(3H)-one), G-clamps such as a substituted phenoxazine cytidine (e.g., 9-(2-aminoethoxy)-H-pyrimido(5,4-(b) (1,4)benzoxazin-2(3H)-one), carbazole cytidine (2H-pyrimido(4,5-b) indol-2-one), pyridoindole cytidine (H-pyrido (3′,2′:4,5)pyrrolo[2,3-d]pyrimidin-2-one).

As used herein, the term “sample” can refer to a composition comprising targets. Suitable samples for analysis by the disclosed methods, devices, and systems include cells, tissues, organs, or organisms.

As used herein, the term “sampling device” or “device” can refer to a device which may take a section of a sample and/or place the section on a substrate. A sample device can refer to, for example, a fluorescence activated cell sorting (FACS) machine, a cell sorter machine, a biopsy needle, a biopsy device, a tissue sectioning device, a microfluidic device, a blade grid, and/or a microtome.

As used herein, the term “solid support” can refer to discrete solid or semi-solid surfaces to which a plurality of barcodes (e.g., stochastic barcodes) may be attached. A solid support may encompass any type of solid, porous, or hollow sphere, ball, bearing, cylinder, or other similar configuration composed of plastic, ceramic, metal, or polymeric material (e.g., hydrogel) onto which a nucleic acid may be immobilized (e.g., covalently or non-covalently). A solid support may comprise a discrete particle that may be spherical (e.g., microspheres) or have a non-spherical or irregular shape, such as cubic, cuboid, pyramidal, cylindrical, conical, oblong, or disc-shaped, and the like. A bead can be non-spherical in shape. A plurality of solid supports spaced in an array may not comprise a substrate. A solid support may be used interchangeably with the term “bead.”

As used herein, the term “stochastic barcode” can refer to a polynucleotide sequence comprising labels of the present disclosure. A stochastic barcode can be a polynucleotide sequence that can be used for stochastic barcoding. Stochastic barcodes can be used to quantify targets within a sample. Stochastic barcodes can be used to control for errors which may occur after a label is associated with a target. For example, a stochastic barcode can be used to assess amplification or sequencing errors. A stochastic barcode associated with a target can be called a stochastic barcode-target or stochastic barcode-tag-target.

As used herein, the term “gene-specific stochastic barcode” can refer to a polynucleotide sequence comprising labels and a target-binding region that is gene-specific. A stochastic barcode can be a polynucleotide sequence that can be used for stochastic barcoding. Stochastic barcodes can be used to quantify targets within a sample. Stochastic barcodes can be used to control for errors which may occur after a label is associated with a target. For example, a stochastic barcode can be used to assess amplification or sequencing errors. A stochastic barcode associated with a target can be called a stochastic barcode-target or stochastic barcode-tag-target.

As used herein, the term “stochastic barcoding” can refer to the random labeling (e.g., barcoding) of nucleic acids. Stochastic barcoding can utilize a recursive Poisson strategy to associate and quantify labels associated with targets. As used herein, the term “stochastic barcoding” can be used interchangeably with “stochastic labeling.”

As used here, the term “target” can refer to a composition which can be associated with a barcode (e.g., a stochastic barcode). Exemplary suitable targets for analysis by the disclosed methods, devices, and systems include oligonucleotides, DNA, RNA, mRNA, microRNA, tRNA, and the like. Targets can be single or double stranded. In some embodiments, targets can be proteins, peptides, or polypeptides. In some embodiments, targets are lipids. As used herein, “target” can be used interchangeably with “species.”

As used herein, the term “reverse transcriptases” can refer to a group of enzymes having reverse transcriptase activity (i.e., that catalyze synthesis of DNA from an RNA template). In general, such enzymes include, but are not limited to, retroviral reverse transcriptase, retrotransposon reverse transcriptase, retroplasmid reverse transcriptases, retron reverse transcriptases, bacterial reverse transcriptases, group II intron-derived reverse transcriptase, and mutants, variants or derivatives thereof. Non-retroviral reverse transcriptases include non-LTR retrotransposon reverse transcriptases, retroplasmid reverse transcriptases, retron reverse transcriptases, and group II intron reverse transcriptases. Examples of group II intron reverse transcriptases include the Lactococcus lactis LI.LtrB intron reverse transcriptase, the Thermosynechococcus elongatus TeI4c intron reverse transcriptase, or the Geobacillus stearothermophilus GsI-IIC intron reverse transcriptase. Other classes of reverse transcriptases can include many classes of non-retroviral reverse transcriptases (i.e., retrons, group II introns, and diversity-generating retroelements among others).

The terms “universal adaptor primer,” “universal primer adaptor” or “universal adaptor sequence” are used interchangeably to refer to a nucleotide sequence that can be used to hybridize to barcodes (e.g., stochastic barcodes) to generate gene-specific barcodes. A universal adaptor sequence can, for example, be a known sequence that is universal across all barcodes used in methods of the disclosure. For example, when multiple targets are being labeled using the methods disclosed herein, each of the target-specific sequences may be linked to the same universal adaptor sequence. In some embodiments, more than one universal adaptor sequences may be used in the methods disclosed herein. For example, when multiple targets are being labeled using the methods disclosed herein, at least two of the target-specific sequences are linked to different universal adaptor sequences. A universal adaptor primer and its complement may be included in two oligonucleotides, one of which comprises a target-specific sequence and the other comprises a barcode. For example, a universal adaptor sequence may be part of an oligonucleotide comprising a target-specific sequence to generate a nucleotide sequence that is complementary to a target nucleic acid. A second oligonucleotide comprising a barcode and a complementary sequence of the universal adaptor sequence may hybridize with the nucleotide sequence and generate a target-specific barcode (e.g., a target-specific stochastic barcode). In some embodiments, a universal adaptor primer has a sequence that is different from a universal PCR primer used in the methods of this disclosure.

### Barcodes

Barcoding, such as stochastic barcoding, has been described in, for example, Fu et al., Proc Natl Acad Sci U.S.A., 2011 May 31,108 (22): 9026-31; US2011/0160078; Fan et al., Science, 2015 Feb. 6, 347 (6222): 1258367; US2015/0299784; and WO2015/031691; the content of each of these, including any supporting or supplemental information or material, is incorporated herein by reference in its entirety. In some embodiments, the barcode disclosed herein can be a stochastic barcode which can be a polynucleotide sequence that may be used to stochastically label (e.g., barcode, tag) a target. Barcodes can be referred to stochastic barcodes if the ratio of the number of different barcode sequences of the stochastic barcodes and the number of occurrence of any of the targets to be labeled can be, or be about, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 50:1, 60:1, 70:1, 80:1, 90:1, 100:1, or a number or a range between any two of these values. A target can be an mRNA species comprising mRNA molecules with identical or nearly identical sequences. Barcodes can be referred to as stochastic barcodes if the ratio of the number of different barcode sequences of the stochastic barcodes and the number of occurrence of any of the targets to be labeled is at least, or is at most, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 50:1, 60:1, 70:1, 80:1, 90:1, or 100:1. Barcode sequences of stochastic barcodes can be referred to as molecular labels.

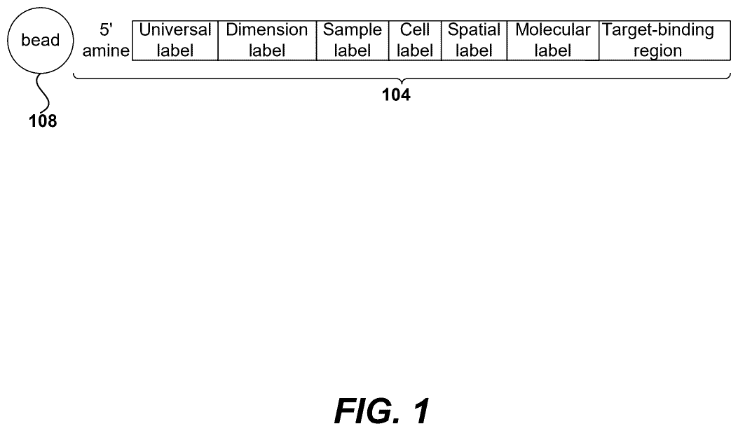

A barcode, for example a stochastic barcode, can comprise one or more labels. Exemplary labels can include a universal label, a cell label, a barcode sequence (e.g., a molecular label), a sample label, a plate label, a spatial label, and/or a pre-spatial label. FIG. 1 illustrates an exemplary barcode 104 with a spatial label. The barcode 104 can comprise a 5′amine that may link the barcode to a solid support 105. The barcode can comprise a universal label, a dimension label, a spatial label, a cell label, and/or a molecular label. The order of different labels (including but not limited to the universal label, the dimension label, the spatial label, the cell label, and the molecule label) in the barcode can vary. For example, as shown in FIG. 1, the universal label may be the 5′-most label, and the molecular label may be the 3′-most label. The spatial label, dimension label, and the cell label may be in any order. In some embodiments, the universal label, the spatial label, the dimension label, the cell label, and the molecular label are in any order. The barcode can comprise a target-binding region. The target-binding region can interact with a target (e.g., target nucleic acid, RNA, mRNA, DNA) in a sample. For example, a target-binding region can comprise an oligo(dT) sequence which can interact with poly(A) tails of mRNAs. In some instances, the labels of the barcode (e.g., universal label, dimension label, spatial label, cell label, and barcode sequence) may be separated by 1, 2, 3, 4, 5, 6, 7, 8, 9, 10, 11, 12, 13, 14, 15, 16, 17, 18, 19, or 20 or more nucleotides.

A label, for example the cell label, can comprise a unique set of nucleic acid sub-sequences of defined length, e.g., seven nucleotides each (equivalent to the number of bits used in some Hamming error correction codes), which can be designed to provide error correction capability. The set of error correction sub-sequences comprise seven nucleotide sequences can be designed such that any pairwise combination of sequences in the set exhibits a defined “genetic distance” (or number of mismatched bases), for example, a set of error correction sub-sequences can be designed to exhibit a genetic distance of three nucleotides. In this case, review of the error correction sequences in the set of sequence data for labeled target nucleic acid molecules (described more fully below) can allow one to detect or correct amplification or sequencing errors. In some embodiments, the length of the nucleic acid sub-sequences used for creating error correction codes can vary, for example, they can be, or be about 1, 2, 3, 4, 5, 6, 7, 8, 9, 10, 15, 20, 30, 31, 40, 50, or a number or a range between any two of these values, nucleotides in length. In some embodiments, nucleic acid sub-sequences of other lengths can be used for creating error correction codes.

The barcode can comprise a target-binding region. The target-binding region can interact with a target in a sample. The target can be, or comprise, ribonucleic acids (RNAs), messenger RNAs (mRNAs), microRNAs, small interfering RNAs (siRNAs), RNA degradation products, RNAs each comprising a poly(A) tail, or any combination thereof. In some embodiments, the plurality of targets can include deoxyribonucleic acids (DNAs).

In some embodiments, a target-binding region can comprise an oligo(dT) sequence which can interact with poly(A) tails of mRNAs. One or more of the labels of the barcode (e.g., the universal label, the dimension label, the spatial label, the cell label, and the barcode sequences (e.g., molecular label)) can be separated by a spacer from another one or two of the remaining labels of the barcode. The spacer can be, for example, 1, 2, 3, 4, 5, 6, 7, 8, 9, 10, 11, 12, 13, 14, 15, 16, 17, 18, 19, or 20, or more nucleotides. In some embodiments, none of the labels of the barcode is separated by spacer.

### Universal Labels

A barcode can comprise one or more universal labels. In some embodiments, the one or more universal labels can be the same for all barcodes in the set of barcodes attached to a given solid support. In some embodiments, the one or more universal labels can be the same for all barcodes attached to a plurality of beads. In some embodiments, a universal label can comprise a nucleic acid sequence that is capable of hybridizing to a sequencing primer. Sequencing primers can be used for sequencing barcodes comprising a universal label. Sequencing primers (e.g., universal sequencing primers) can comprise sequencing primers associated with high-throughput sequencing platforms. In some embodiments, a universal label can comprise a nucleic acid sequence that is capable of hybridizing to a PCR primer. In some embodiments, the universal label can comprise a nucleic acid sequence that is capable of hybridizing to a sequencing primer and a PCR primer. The nucleic acid sequence of the universal label that is capable of hybridizing to a sequencing or PCR primer can be referred to as a primer binding site. A universal label can comprise a sequence that can be used to initiate transcription of the barcode. A universal label can comprise a sequence that can be used for extension of the barcode or a region within the barcode. A universal label can be, or be about, 1, 2, 3, 4, 5, 10, 15, 20, 25, 30, 35, 40, 45, 50, or a number or a range between any two of these values, nucleotides in length. For example, a universal label can comprise at least about 10 nucleotides. A universal label can be at least, or be at most, 1, 2, 3, 4, 5, 10, 15, 20, 25, 30, 35, 40, 45, 50, 100, 200, or 300 nucleotides in length. In some embodiments, a cleavable linker or modified nucleotide can be part of the universal label sequence to enable the barcode to be cleaved off from the support.

### Dimension Labels

A barcode can comprise one or more dimension labels. In some embodiments, a dimension label can comprise a nucleic acid sequence that provides information about a dimension in which the labeling (e.g., stochastic labeling) occurred. For example, a dimension label can provide information about the time at which a target was barcoded. A dimension label can be associated with a time of barcoding (e.g., stochastic barcoding) in a sample. A dimension label can be activated at the time of labeling. Different dimension labels can be activated at different times. The dimension label provides information about the order in which targets, groups of targets, and/or samples were barcoded. For example, a population of cells can be barcoded at the GO phase of the cell cycle. The cells can be pulsed again with barcodes (e.g., stochastic barcodes) at the Gl phase of the cell cycle. The cells can be pulsed again with barcodes at the S phase of the cell cycle, and so on. Barcodes at each pulse (e.g., each phase of the cell cycle), can comprise different dimension labels. In this way, the dimension label provides information about which targets were labelled at which phase of the cell cycle. Dimension labels can interrogate many different biological times. Exemplary biological times can include, but are not limited to, the cell cycle, transcription (e.g., transcription initiation), and transcript degradation. In another example, a sample (e.g., a cell, a population of cells) can be labeled before and/or after treatment with a drug and/or therapy. The changes in the number of copies of distinct targets can be indicative of the sample's response to the drug and/or therapy.

A dimension label can be activatable. An activatable dimension label can be activated at a specific time point. The activatable label can be, for example, constitutively activated (e.g., not turned off). The activatable dimension label can be, for example, reversibly activated (e.g., the activatable dimension label can be turned on and turned off). The dimension label can be, for example, reversibly activatable at least 1, 2, 3, 4, 5, 6, 7, 8, 9, 10, or more times. The dimension label can be reversibly activatable, for example, at least 1, 2, 3, 4, 5, 6, 7, 8, 9, 10 or more times. In some embodiments, the dimension label can be activated with fluorescence, light, a chemical event (e.g., cleavage, ligation of another molecule, addition of modifications (e.g., pegylated, sumoylated, acetylated, methylated, deacetylated, demethylated), a photochemical event (e.g., photocaging), and introduction of a non-natural nucleotide.

The dimension label can, in some embodiments, be identical for all barcodes (e.g., stochastic barcodes) attached to a given solid support (e.g., a bead), but different for different solid supports (e.g., beads). In some embodiments, at least 60%, 70%, 80%, 85%, 90%, 95%, 97%, 99% or 100%, of barcodes on the same solid support can comprise the same dimension label. In some embodiments, at least 60% of barcodes on the same solid support can comprise the same dimension label. In some embodiments, at least 95% of barcodes on the same solid support can comprise the same dimension label.

There can be as many as 10⁶ or more unique dimension label sequences represented in a plurality of solid supports (e.g., beads). A dimension label can be, or be about 1, 2, 3, 4, 5, 10, 15, 20, 25, 30, 35, 40, 45, 50, or a number or a range between any two of these values, nucleotides in length. A dimension label can be at least, or be at most, 1, 2, 3, 4, 5, 10, 15, 20, 25, 30, 35, 40, 45, 50, 100, 200, or 300, nucleotides in length. A dimension label can comprise between about 5 to about 200 nucleotides. A dimension label can comprise between about 10 to about 150 nucleotides. A dimension label can comprise between about 20 to about 125 nucleotides in length.

### Spatial Labels

A barcode can comprise one or more spatial labels. In some embodiments, a spatial label can comprise a nucleic acid sequence that provides information about the spatial orientation of a target molecule which is associated with the barcode. A spatial label can be associated with a coordinate in a sample. The coordinate can be a fixed coordinate. For example, a coordinate can be fixed in reference to a substrate. A spatial label can be in reference to a two or three-dimensional grid. A coordinate can be fixed in reference to a landmark. The landmark can be identifiable in space. A landmark can be a structure which can be imaged. A landmark can be a biological structure, for example an anatomical landmark. A landmark can be a cellular landmark, for instance an organelle. A landmark can be a non-natural landmark such as a structure with an identifiable identifier such as a color code, bar code, magnetic property, fluorescents, radioactivity, or a unique size or shape. A spatial label can be associated with a physical partition (e.g., A well, a container, or a droplet). In some embodiments, multiple spatial labels are used together to encode one or more positions in space.

The spatial label can be identical for all barcodes attached to a given solid support (e.g., a bead), but different for different solid supports (e.g., beads). In some embodiments, the percentage of barcodes on the same solid support comprising the same spatial label can be, or be about, 60%, 70%, 80%, 85%, 90%, 95%, 97%, 99%, 100%, or a number or a range between any two of these values. In some embodiments, the percentage of barcodes on the same solid support comprising the same spatial label can be at least, or be at most, 60%, 70%, 80%, 85%, 90%, 95%, 97%, 99%, or 100%. In some embodiments, at least 60% of barcodes on the same solid support can comprise the same spatial label. In some embodiments, at least 95% of barcodes on the same solid support can comprise the same spatial label.

There can be as many as 10⁶ or more unique spatial label sequences represented in a plurality of solid supports (e.g., beads). A spatial label can be, or be about, 1, 2, 3, 4, 5, 10, 15, 20, 25, 30, 35, 40, 45, 50, or a number or a range between any two of these values, nucleotides in length. A spatial label can be at least or at most 1, 2, 3, 4, 5, 10, 15, 20, 25, 30, 35, 40, 45, 50, 100, 200, or 300 nucleotides in length. A spatial label can comprise between about 5 to about 200 nucleotides. A spatial label can comprise between about 10 to about 150 nucleotides. A spatial label can comprise between about 20 to about 125 nucleotides in length.

### Cell Labels

A barcode (e.g., a stochastic barcode) can comprise one or more cell labels. In some embodiments, a cell label can comprise a nucleic acid sequence that provides information for determining which target nucleic acid originated from which cell. In some embodiments, the cell label is identical for all barcodes attached to a given solid support (e.g., a bead), but different for different solid supports (e.g., beads). In some embodiments, the percentage of barcodes on the same solid support comprising the same cell label can be, or be about 60%, 70%, 80%, 85%, 90%, 95%, 97%, 99%, 100%, or a number or a range between any two of these values. In some embodiments, the percentage of barcodes on the same solid support comprising the same cell label can be, or be about 60%, 70%, 80%, 85%, 90%, 95%, 97%, 99%, or 100%. For example, at least 60% of barcodes on the same solid support can comprise the same cell label. As another example, at least 95% of barcodes on the same solid support can comprise the same cell label.

There can be as many as 10⁶ or more unique cell label sequences represented in a plurality of solid supports (e.g., beads). A cell label can be, or be about, 1, 2, 3, 4, 5, 10, 15, 20, 25, 30, 35, 40, 45, 50, or a number or a range between any two of these values, nucleotides in length. A cell label can be at least, or be at most, 1, 2, 3, 4, 5, 10, 15, 20, 25, 30, 35, 40, 45, 50, 100, 200, or 300 nucleotides in length. For example, a cell label can comprise between about 5 to about 200 nucleotides. As another example, a cell label can comprise between about 10 to about 150 nucleotides. As yet another example, a cell label can comprise between about 20 to about 125 nucleotides in length.

### Barcode Sequences

A barcode can comprise one or more barcode sequences. In some embodiments, a barcode sequence can comprise a nucleic acid sequence that provides identifying information for the specific type of target nucleic acid species hybridized to the barcode. A barcode sequence can comprise a nucleic acid sequence that provides a counter (e.g., that provides a rough approximation) for the specific occurrence of the target nucleic acid species hybridized to the barcode (e.g., target-binding region).

In some embodiments, a diverse set of barcode sequences are attached to a given solid support (e.g., a bead). In some embodiments, there can be, or be about, 10², 10³, 10⁴, 10⁵, 10⁶, 10⁷, 10⁸, 10⁹, or a number or a range between any two of these values, unique molecular label sequences. For example, a plurality of barcodes can comprise about 6561 barcodes sequences with distinct sequences. As another example, a plurality of barcodes can comprise about 65536 barcode sequences with distinct sequences. In some embodiments, there can be at least, or be at most, 10², 10³, 10⁴, 10⁵, 10⁶, 10⁷, 10⁸, or 10⁹, unique barcode sequences. The unique molecular label sequences can be attached to a given solid support (e.g., a bead). In some embodiments, the unique molecular label sequence is partially or entirely encompassed by a particle (e.g., a hydrogel bead).

The length of a barcode can be different in different implementations. For example, a barcode can be, or be about, 1, 2, 3, 4, 5, 10, 15, 20, 25, 30, 35, 40, 45, 50, or a number or a range between any two of these values, nucleotides in length. As another example, a barcode can be at least, or be at most, 1, 2, 3, 4, 5, 10, 15, 20, 25, 30, 35, 40, 45, 50, 100, 200, or 300 nucleotides in length.

### Molecular Labels

A barcode (e.g., a stochastic barcode) can comprise one or more molecular labels. Molecular labels can include barcode sequences. In some embodiments, a molecular label can comprise a nucleic acid sequence that provides identifying information for the specific type of target nucleic acid species hybridized to the barcode. A molecular label can comprise a nucleic acid sequence that provides a counter for the specific occurrence of the target nucleic acid species hybridized to the barcode (e.g., target-binding region).

In some embodiments, a diverse set of molecular labels are attached to a given solid support (e.g., a bead). In some embodiments, there can be, or be about, 10², 10³, 10⁴, 10⁵, 10⁶, 10⁷, 10⁸, 10⁹, or a number or a range between any two of these values, of unique molecular label sequences. For example, a plurality of barcodes can comprise about 6561 molecular labels with distinct sequences. As another example, a plurality of barcodes can comprise about 65536 molecular labels with distinct sequences. In some embodiments, there can be at least, or be at most, 10², 10³, 10⁴, 10⁵, 10⁶, 10⁷, 10⁸, or 10⁹, unique molecular label sequences. Barcodes with unique molecular label sequences can be attached to a given solid support (e.g., a bead).

For barcoding (e.g., stochastic barcoding) using a plurality of stochastic barcodes, the ratio of the number of different molecular label sequences and the number of occurrence of any of the targets can be, or be about, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 50:1, 60:1, 70:1, 80:1, 90:1, 100:1, or a number or a range between any two of these values. A target can be an mRNA species comprising mRNA molecules with identical or nearly identical sequences. In some embodiments, the ratio of the number of different molecular label sequences and the number of occurrence of any of the targets is at least, or is at most, 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 30:1, 40:1, 50:1, 60:1, 70:1, 80:1, 90:1, or 100:1.

A molecular label can be, or be about, 1, 2, 3, 4, 5, 10, 15, 20, 25, 30, 35, 40, 45, 50, or a number or a range between any two of these values, nucleotides in length. A molecular label can be at least, or be at most, 1, 2, 3, 4, 5, 10, 15, 20, 25, 30, 35, 40, 45, 50, 100, 200, or 300 nucleotides in length.

### Target-Binding Region

A barcode can comprise one or more target binding regions, such as capture probes. In some embodiments, a target-binding region can hybridize with a target of interest. In some embodiments, the target binding regions can comprise a nucleic acid sequence that hybridizes specifically to a target (e.g., target nucleic acid, target molecule, e.g., a cellular nucleic acid to be analyzed), for example to a specific gene sequence. In some embodiments, a target binding region can comprise a nucleic acid sequence that can attach (e.g., hybridize) to a specific location of a specific target nucleic acid. In some embodiments, the target binding region can comprise a nucleic acid sequence that is capable of specific hybridization to a restriction enzyme site overhang (e.g., an EcoRI sticky-end overhang). The barcode can then ligate to any nucleic acid molecule comprising a sequence complementary to the restriction site overhang.

In some embodiments, a target binding region can comprise a non-specific target nucleic acid sequence. A non-specific target nucleic acid sequence can refer to a sequence that can bind to multiple target nucleic acids, independent of the specific sequence of the target nucleic acid. For example, target binding region can comprise a random multimer sequence, a poly(dA) sequence, a poly(dT) sequence, a poly(dG) sequence, a poly(dC) sequence, or a combination thereof. For example, the target binding region can be an oligo(dT) sequence that hybridizes to the poly(A) sitail on mRNA molecules. A random multimer sequence can be, for example, a random dimer, trimer, quatramer, pentamer, hexamer, septamer, octamer, nonamer, decamer, or higher multimer sequence of any length. In some embodiments, the target binding region is the same for all barcodes attached to a given bead. In some embodiments, the target binding regions for the plurality of barcodes attached to a given bead can comprise two or more different target binding sequences. A target binding region can be, or be about, 5, 10, 15, 20, 25, 30, 35, 40, 45, 50, or a number or a range between any two of these values, nucleotides in length. A target binding region can be at most about 5, 10, 15, 20, 25, 30, 35, 40, 45, 50 or more nucleotides in length. For example, an mRNA molecule can be reverse transcribed using a reverse transcriptase, such as Moloney murine leukemia virus (MMLV) reverse transcriptase, to generate a cDNA molecule with a poly(dC) tail. A barcode can include a target binding region with a poly(dG) tail. Upon base pairing between the poly(dG) tail of the barcode and the poly(dC) tail of the cDNA molecule, the reverse transcriptase switches template strands, from cellular RNA molecule to the barcode, and continues replication to the 5′ end of the barcode. By doing so, the resulting cDNA molecule contains the sequence of the barcode (such as the molecular label) on the 3′ end of the cDNA molecule.

In some embodiments, a target-binding region can comprise an oligo(dT) which can hybridize with mRNAs comprising polyadenylated ends. A target-binding region can be gene-specific. For example, a target-binding region can be configured to hybridize to a specific region of a target. A target-binding region can be, or be about, 1, 2, 3, 4, 5, 6, 7, 8, 9, 10, 11, 12, 13, 14, 15, 16, 17, 18, 19, 20, 21, 22, 23, 24, 25, 26 27, 28, 29, 30, or a number or a range between any two of these values, nucleotides in length. A target-binding region can be at least, or be at most, 1, 2, 3, 4, 5, 6, 7, 8, 9, 10, 11, 12, 13, 14, 15, 16, 17, 18, 19, 20, 21, 22, 23, 24, 25, 26 27, 28, 29, or 30, nucleotides in length. A target-binding region can be about 5-30 nucleotides in length. When a barcode comprises a gene-specific target-binding region, the barcode can be referred to herein as a gene-specific barcode.

### Orientation Property

A stochastic barcode (e.g., a stochastic barcode) can comprise one or more orientation properties which can be used to orient (e.g., align) the barcodes. A barcode can comprise a moiety for isoelectric focusing. Different barcodes can comprise different isoelectric focusing points. When these barcodes are introduced to a sample, the sample can undergo isoelectric focusing in order to orient the barcodes into a known way. In this way, the orientation property can be used to develop a known map of barcodes in a sample. Exemplary orientation properties can include, electrophoretic mobility (e.g., based on size of the barcode), isoelectric point, spin, conductivity, and/or self-assembly. For example, barcodes with an orientation property of self-assembly, can self-assemble into a specific orientation (e.g., nucleic acid nanostructure) upon activation.

### Affinity Property

A barcode (e.g., a stochastic barcode) can comprise one or more affinity properties. For example, a spatial label can comprise an affinity property. An affinity property can include a chemical and/or biological moiety that can facilitate binding of the barcode to another entity (e.g., cell receptor). For example, an affinity property can comprise an antibody, for example, an antibody specific for a specific moiety (e.g., receptor) on a sample. In some embodiments, the antibody can guide the barcode to a specific cell type or molecule. Targets at and/or near the specific cell type or molecule can be labeled (e.g., stochastically labeled). The affinity property can, in some embodiments, provide spatial information in addition to the nucleotide sequence of the spatial label because the antibody can guide the barcode to a specific location. The antibody can be a therapeutic antibody, for example a monoclonal antibody or a polyclonal antibody. The antibody can be humanized or chimeric. The antibody can be a naked antibody or a fusion antibody.

The antibody can be a full-length (i.e., naturally occurring or formed by normal immunoglobulin gene fragment recombinatorial processes) immunoglobulin molecule (e.g., an IgG antibody) or an immunologically active (i.e., specifically binding) portion of an immunoglobulin molecule, like an antibody fragment.

The antibody fragment can be, for example, a portion of an antibody such as F(ab′)2, Fab′, Fab, Fv, sFv and the like. In some embodiments, the antibody fragment can bind with the same antigen that is recognized by the full-length antibody. The antibody fragment can include isolated fragments consisting of the variable regions of antibodies, such as the “Fv” fragments consisting of the variable regions of the heavy and light chains and recombinant single chain polypeptide molecules in which light and heavy variable regions are connected by a peptide linker (“scFv proteins”). Exemplary antibodies can include, but are not limited to, antibodies for cancer cells, antibodies for viruses, antibodies that bind to cell surface receptors (CD8, CD34, CD45), and therapeutic antibodies.

### Universal Adaptor Primer

A barcode can comprise one or more universal adaptor primers. For example, a gene-specific barcode, such as a gene-specific stochastic barcode, can comprise a universal adaptor primer. A universal adaptor primer can refer to a nucleotide sequence that is universal across all barcodes. A universal adaptor primer can be used for building gene-specific barcodes. A universal adaptor primer can be, or be about, 1, 2, 3, 4, 5, 6, 7, 8, 9, 10, 11, 12, 13, 14, 15, 16, 17, 18, 19, 20, 21, 22, 23, 24, 25, 26 27, 28, 29, 30, or a number or a range between any two of these nucleotides in length. A universal adaptor primer can be at least, or be at most, 1, 2, 3, 4, 5, 6, 7, 8, 9, 10, 11, 12, 13, 14, 15, 16, 17, 18, 19, 20, 21, 22, 23, 24, 25, 26 27, 28, 29, or 30 nucleotides in length. A universal adaptor primer can be from 5-30 nucleotides in length.

### Linker

When a barcode comprises more than one of a type of label (e.g., more than one cell label or more than one barcode sequence, such as one molecular label), the labels may be interspersed with a linker label sequence. A linker label sequence can be at least about 5, 10, 15, 20, 25, 30, 35, 40, 45, 50 or more nucleotides in length. A linker label sequence can be at most about 5, 10, 15, 20, 25, 30, 35, 40, 45, 50 or more nucleotides in length. In some instances, a linker label sequence is 12 nucleotides in length. A linker label sequence can be used to facilitate the synthesis of the barcode. The linker label can comprise an error-correcting (e.g., Hamming) code.

### Solid Supports

Barcodes, such as stochastic barcodes, disclosed herein can, in some embodiments, be associated with a solid support. The solid support can be, for example, a synthetic particle. In some embodiments, some or all of the barcode sequences, such as molecular labels for stochastic barcodes (e.g., the first barcode sequences) of a plurality of barcodes (e.g., the first plurality of barcodes) on a solid support differ by at least one nucleotide. The cell labels of the barcodes on the same solid support can be the same. The cell labels of the barcodes on different solid supports can differ by at least one nucleotide. For example, first cell labels of a first plurality of barcodes on a first solid support can have the same sequence, and second cell labels of a second plurality of barcodes on a second solid support can have the same sequence. The first cell labels of the first plurality of barcodes on the first solid support and the second cell labels of the second plurality of barcodes on the second solid support can differ by at least one nucleotide. A cell label can be, for example, about 5-20 nucleotides long. A barcode sequence can be, for example, about 5-20 nucleotides long. The synthetic particle can be, for example, a bead.

The bead can be, for example, a silica gel bead, a controlled pore glass bead, a magnetic bead, a Dynabead, a Sephadex/Sepharose bead, a cellulose bead, a polystyrene bead, or any combination thereof. The bead can comprise a material such as polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, or any combination thereof.

In some embodiments, the bead can be a polymeric bead, for example a deformable bead or a gel bead, functionalized with barcodes or stochastic barcodes (such as gel beads from 10X Genomics (San Francisco, CA). In some implementation, a gel bead can comprise a polymer based gels. Gel beads can be generated, for example, by encapsulating one or more polymeric precursors into droplets. Upon exposure of the polymeric precursors to an accelerator (e.g., tetramethylethylenediamine (TEMED)), a gel bead may be generated.

The particle can be disruptable (e.g., dissolvable, degradable). For example, the polymeric bead can dissolve, melt, or degrade, for example, under a desired condition. The desired condition can include an environmental condition. The desired condition may result in the polymeric bead dissolving, melting, or degrading in a controlled manner. A gel bead may dissolve, melt, or degrade due to a chemical stimulus, a physical stimulus, a biological stimulus, a thermal stimulus, a magnetic stimulus, an electric stimulus, a light stimulus, or any combination thereof.

Analytes and/or reagents, such as oligonucleotide barcodes, for example, may be coupled/immobilized to the interior surface of a gel bead (e.g., the interior accessible via diffusion of an oligonucleotide barcode and/or materials used to generate an oligonucleotide barcode) and/or the outer surface of a gel bead or any other microcapsule described herein. Coupling/immobilization may be via any form of chemical bonding (e.g., covalent bond, ionic bond) or physical phenomena (e.g., Van der Waals forces, dipole-dipole interactions, etc.). In some embodiments, coupling/immobilization of a reagent to a gel bead or any other microcapsule described herein may be reversible, such as, for example, via a labile moiety (e.g., via a chemical cross-linker, including chemical cross-linkers described herein). Upon application of a stimulus, the labile moiety may be cleaved and the immobilized reagent set free. In some embodiments, the labile moiety is a disulfide bond. For example, in the case where an oligonucleotide barcode is immobilized to a gel bead via a disulfide bond, exposure of the disulfide bond to a reducing agent can cleave the disulfide bond and free the oligonucleotide barcode from the bead. The labile moiety may be included as part of a gel bead or microcapsule, as part of a chemical linker that links a reagent or analyte to a gel bead or microcapsule, and/or as part of a reagent or analyte. In some embodiments, at least one barcode of the plurality of barcodes can be immobilized on the particle, partially immobilized on the particle, enclosed in the particle, partially enclosed in the particle, or any combination thereof.

In some embodiments, a gel bead can comprise a wide range of different polymers including but not limited to: polymers, heat sensitive polymers, photosensitive polymers, magnetic polymers, pH sensitive polymers, salt-sensitive polymers, chemically sensitive polymers, polyelectrolytes, polysaccharides, peptides, proteins, and/or plastics. Polymers may include but are not limited to materials such as poly(N-isopropylacrylamide) (PNIPAAm), poly(styrene sulfonate) (PSS), poly(allyl amine) (PAAm), poly(acrylic acid) (PAA), poly(ethylene imine) (PEI), poly(diallyldimethyl-ammonium chloride) (PDADMAC), poly(pyrolle) (PPy), poly(vinylpyrrolidone) (PVPON), poly(vinyl pyridine) (PVP), poly(methacrylic acid) (PMAA), poly(methyl methacrylate) (PMMA), polystyrene (PS), poly(tetrahydrofuran) (PTHF), poly(phthaladehyde) (PTHF), poly(hexyl viologen) (PHV), poly(L-lysine) (PLL), poly(L-arginine) (PARG), poly(lactic-co-glycolic acid) (PLGA).

Numerous chemical stimuli can be used to trigger the disruption, dissolution, or degradation of the beads. Examples of these chemical changes may include, but are not limited to pH-mediated changes to the bead wall, disintegration of the bead wall via chemical cleavage of crosslink bonds, triggered depolymerization of the bead wall, and bead wall switching reactions. Bulk changes may also be used to trigger disruption of the beads.

Bulk or physical changes to the microcapsule through various stimuli also offer many advantages in designing capsules to release reagents. Bulk or physical changes occur on a macroscopic scale, in which bead rupture is the result of mechano-physical forces induced by a stimulus. These processes may include, but are not limited to pressure induced rupture, bead wall melting, or changes in the porosity of the bead wall.

Biological stimuli may also be used to trigger disruption, dissolution, or degradation of beads. Generally, biological triggers resemble chemical triggers, but many examples use biomolecules, or molecules commonly found in living systems such as enzymes, peptides, saccharides, fatty acids, nucleic acids and the like. For example, beads may comprise polymers with peptide cross-links that are sensitive to cleavage by specific proteases. More specifically, one example may comprise a microcapsule comprising GFLGK peptide cross links. Upon addition of a biological trigger such as the protease Cathepsin B, the peptide cross links of the shell well are cleaved and the contents of the beads are released. In other cases, the proteases may be heat-activated. In another example, beads comprise a shell wall comprising cellulose. Addition of the hydrolytic enzyme chitosan serves as biologic trigger for cleavage of cellulosic bonds, depolymerization of the shell wall, and release of its inner contents.

The beads may also be induced to release their contents upon the application of a thermal stimulus. A change in temperature can cause a variety changes to the beads. A change in heat may cause melting of a bead such that the bead wall disintegrates. In other cases, the heat may increase the internal pressure of the inner components of the bead such that the bead ruptures or explodes. In still other cases, the heat may transform the bead into a shrunken dehydrated state. The heat may also act upon heat-sensitive polymers within the wall of a bead to cause disruption of the bead.

Inclusion of magnetic nanoparticles to the bead wall of microcapsules may allow triggered rupture of the beads as well as guide the beads in an array. A device of this disclosure may comprise magnetic beads for either purpose. In one example, incorporation of Fe₃O₄ nanoparticles into polyelectrolyte containing beads triggers rupture in the presence of an oscillating magnetic field stimulus.

A bead may also be disrupted, dissolved, or degraded as the result of electrical stimulation. Similar to magnetic particles described in the previous section, electrically sensitive beads can allow for both triggered rupture of the beads as well as other functions such as alignment in an electric field, electrical conductivity or redox reactions. In one example, beads containing electrically sensitive material are aligned in an electric field such that release of inner reagents can be controlled. In other examples, electrical fields may induce redox reactions within the bead wall itself that may increase porosity.

A light stimulus may also be used to disrupt the beads. Numerous light triggers are possible and may include systems that use various molecules such as nanoparticles and chromophores capable of absorbing photons of specific ranges of wavelengths. For example, metal oxide coatings can be used as capsule triggers. UV irradiation of polyelectrolyte capsules coated with SiO₂ may result in disintegration of the bead wall. In yet another example, photo switchable materials such as azobenzene groups may be incorporated in the bead wall. Upon the application of UV or visible light, chemicals such as these undergo a reversible cis-to-trans isomerization upon absorption of photons. In this aspect, incorporation of photon switches result in a bead wall that may disintegrate or become more porous upon the application of a light trigger.

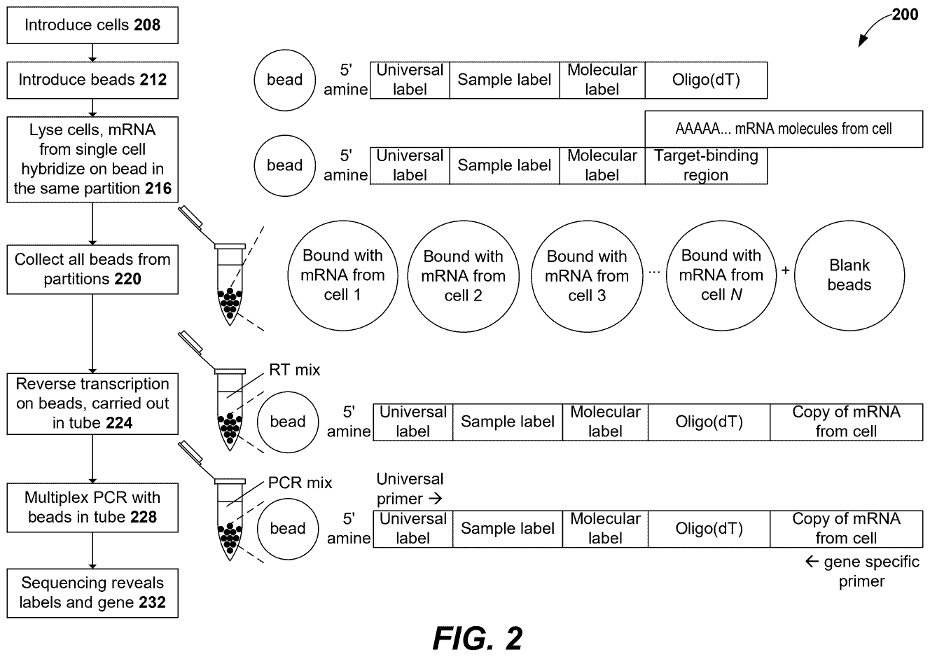

For example, in a non-limiting example of barcoding (e.g., stochastic barcoding) illustrated in FIG. 2, after introducing cells such as single cells onto a plurality of microwells of a microwell array at block 208, beads can be introduced onto the plurality of microwells of the microwell array at block 212. Each microwell can comprise one bead. The beads can comprise a plurality of barcodes. A barcode can comprise a 5′ amine region attached to a bead. The barcode can comprise a universal label, a barcode sequence (e.g., a molecular label), a target-binding region, or any combination thereof.

The barcodes disclosed herein can be associated with (e.g., attached to) a solid support (e.g., a bead). The barcodes associated with a solid support can each comprise a barcode sequence selected from a group comprising at least 100 or 1000 barcode sequences with unique sequences. In some embodiments, different barcodes associated with a solid support can comprise barcode with different sequences. In some embodiments, a percentage of barcodes associated with a solid support comprises the same cell label. For example, the percentage can be, or be about 60%, 70%, 80%, 85%, 90%, 95%, 97%, 99%, 100%, or a number or a range between any two of these values. As another example, the percentage can be at least, or be at most 60%, 70%, 80%, 85%, 90%, 95%, 97%, 99%, or 100%. In some embodiments, barcodes associated with a solid support can have the same cell label. The barcodes associated with different solid supports can have different cell labels selected from a group comprising at least 100 or 1000 cell labels with unique sequences.

The barcodes disclosed herein can be associated to (e.g., attached to) a solid support (e.g., a bead). In some embodiments, barcoding the plurality of targets in the sample can be performed with a solid support including a plurality of synthetic particles associated with the plurality of barcodes. In some embodiments, the solid support can include a plurality of synthetic particles associated with the plurality of barcodes. The spatial labels of the plurality of barcodes on different solid supports can differ by at least one nucleotide. The solid support can, for example, include the plurality of barcodes in two dimensions or three dimensions. The synthetic particles can be beads. The beads can be silica gel beads, controlled pore glass beads, magnetic beads, Dynabeads, Sephadex/Sepharose beads, cellulose beads, polystyrene beads, or any combination thereof. The solid support can include a polymer, a matrix, a hydrogel, a needle array device, an antibody, or any combination thereof. In some embodiments, the solid supports can be free floating. In some embodiments, the solid supports can be embedded in a semi-solid or solid array. The barcodes may not be associated with solid supports. The barcodes can be individual nucleotides. The barcodes can be associated with a substrate.

As used herein, the terms “tethered,” “attached,” and “immobilized,” are used interchangeably, and can refer to covalent or non-covalent means for attaching barcodes to a solid support. Any of a variety of different solid supports can be used as solid supports for attaching pre-synthesized barcodes or for in situ solid-phase synthesis of barcode.

In some embodiments, the solid support is a bead. The bead can comprise one or more types of solid, porous, or hollow sphere, ball, bearing, cylinder, or other similar configuration which a nucleic acid can be immobilized (e.g., covalently or non-covalently). The bead can be, for example, composed of plastic, ceramic, metal, polymeric material, or any combination thereof. A bead can be, or comprise, a discrete particle that is spherical (e.g., microspheres) or have a non-spherical or irregular shape, such as cubic, cuboid, pyramidal, cylindrical, conical, oblong, or disc-shaped, and the like. In some embodiments, a bead can be non-spherical in shape.

Beads can comprise a variety of materials including, but not limited to, paramagnetic materials (e.g., magnesium, molybdenum, lithium, and tantalum), superparamagnetic materials (e.g., ferrite (Fe₃O₄; magnetite) nanoparticles), ferromagnetic materials (e.g., iron, nickel, cobalt, some alloys thereof, and some rare earth metal compounds), ceramic, plastic, glass, polystyrene, silica, methylstyrene, acrylic polymers, titanium, latex, Sepharose, agarose, hydrogel, polymer, cellulose, nylon, or any combination thereof.

In some embodiments, the bead (e.g., the bead to which the labels are attached) is a hydrogel bead. In some embodiments, the bead comprises hydrogel.

Some embodiments disclosed herein include one or more particles (for example, beads). Each of the particles can comprise a plurality of oligonucleotides (e.g., barcodes). Each of the plurality of oligonucleotides can comprise a barcode sequence (e.g., a molecular label sequence), a cell label, and a target-binding region (e.g., an oligo(dT) sequence, a gene-specific sequence, a random multimer, or a combination thereof). The cell label sequence of each of the plurality of oligonucleotides can be the same. The cell label sequences of oligonucleotides on different particles can be different such that the oligonucleotides on different particles can be identified. The number of different cell label sequences can be different in different implementations. In some embodiments, the number of cell label sequences can be, or be about 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 106, 107, 108, 109, a number or a range between any two of these values, or more. In some embodiments, the number of cell label sequences can be at least, or be at most 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 106, 107, 108, or 109. In some embodiments, no more than 1, 2, 3, 4, 5, 6, 7, 8, 9, 10, 20, 30, 40, 50, 60, 70, 80, 90, 100, 200, 300, 400, 500, 600, 700, 800, 900, 1000, or more of the plurality of the particles include oligonucleotides with the same cell sequence. In some embodiment, the plurality of particles that include oligonucleotides with the same cell sequence can be at most 0.1%, 0.2%, 0.3%, 0.4%, 0.5%, 0.6%, 0.7%, 0.8%, 0.9%, 1%, 2%, 3%, 4%, 5%, 6%, 7%, 8%, 9%, 10%, or more. In some embodiments, none of the plurality of the particles has the same cell label sequence.

The plurality of oligonucleotides on each particle can comprise different barcode sequences (e.g., molecular labels). In some embodiments, the number of barcode sequences can be, or be about 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 106, 107, 108, 109, or a number or a range between any two of these values. In some embodiments, the number of barcode sequences can be at least, or be at most 10, 100, 200, 300, 400, 500, 600, 700, 800, 900, 1000, 2000, 3000, 4000, 5000, 6000, 7000, 8000, 9000, 10000, 20000, 30000, 40000, 50000, 60000, 70000, 80000, 90000, 100000, 106, 107, 108, or 109. For example, at least 100 of the plurality of oligonucleotides comprise different barcode sequences. As another example, in a single particle, at least 100, 500, 1000, 5000, 10000, 15000, 20000, 50000, a number or a range between any two of these values, or more of the plurality of oligonucleotides comprise different barcode sequences. Some embodiments provide a plurality of the particles comprising barcodes. In some embodiments, the ratio of an occurrence (or a copy or a number) of a target to be labeled and the different barcode sequences can be at least 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:30, 1:40, 1:50, 1:60, 1:70, 1:80, 1:90, or more. In some embodiments, each of the plurality of oligonucleotides further comprises a sample label, a universal label, or both. The particle can be, for example, a nanoparticle or microparticle.

The size of the beads can vary. For example, the diameter of the bead can range from 0.1 micrometer to 50 micrometer. In some embodiments, the diameter of the bead can be, or be about, 0.1, 0.5, 1, 2, 3, 4, 5, 6, 7, 8, 9, 10, 20, 30, 40, 50 micrometer, or a number or a range between any two of these values.

The diameter of the bead can be related to the diameter of the wells of the substrate. In some embodiments, the diameter of the bead can be, or be about, 10%, 20%, 30%, 40%, 50%, 60%, 70%, 80%, 90%, 100%, or a number or a range between any two of these values, longer or shorter than the diameter of the well. The diameter of the beads can be related to the diameter of a cell (e.g., a single cell entrapped by a well of the substrate). In some embodiments, the diameter of the bead can be at least, or be at most, 10%, 20%, 30%, 40%, 50%, 60%, 70%, 80%, 90%, or 100% longer or shorter than the diameter of the well. The diameter of the beads can be related to the diameter of a cell (e.g., a single cell entrapped by a well of the substrate). In some embodiments, the diameter of the bead can be, or be about, 10%, 20%, 30%, 40%, 50%, 60%, 70%, 80%, 90%, 100%, 150%, 200%, 250%, 300%, or a number or a range between any two of these values, longer or shorter than the diameter of the cell. In some embodiments, the diameter of the beads can be at least, or be at most, 10%, 20%, 30%, 40%, 50%, 60%, 70%, 80%, 90%, 100%, 150%, 200%, 250%, or 300% longer or shorter than the diameter of the cell.

A bead can be attached to and/or embedded in a substrate. A bead can be attached to and/or embedded in a gel, hydrogel, polymer and/or matrix. The spatial position of a bead within a substrate (e.g., gel, matrix, scaffold, or polymer) can be identified using the spatial label present on the barcode on the bead which can serve as a location address.

Examples of beads can include, but are not limited to, streptavidin beads, agarose beads, magnetic beads, Dynabeads®, MACS® microbeads, antibody conjugated beads (e.g., anti-immunoglobulin microbeads), protein A conjugated beads, protein G conjugated beads, protein A/G conjugated beads, protein L conjugated beads, oligo(dT) conjugated beads, silica beads, silica-like beads, anti-biotin microbeads, anti-fluorochrome microbeads, and BcMag™ Carboxyl-Terminated Magnetic Beads.

A bead can be associated with (e.g., impregnated with) quantum dots or fluorescent dyes to make it fluorescent in one fluorescence optical channel or multiple optical channels. A bead can be associated with iron oxide or chromium oxide to make it paramagnetic or ferromagnetic. Beads can be identifiable. For example, a bead can be imaged using a camera. A bead can have a detectable code associated with the bead. For example, a bead can comprise a barcode. A bead can change size, for example, due to swelling in an organic or inorganic solution. A bead can be hydrophobic. A bead can be hydrophilic. A bead can be biocompatible.

A solid support (e.g., a bead) can be visualized. The solid support can comprise a visualizing tag (e.g., fluorescent dye). A solid support (e.g., a bead) can be etched with an identifier (e.g., a number). The identifier can be visualized through imaging the beads.

A solid support can comprise an insoluble, semi-soluble, or insoluble material. A solid support can be referred to as “functionalized” when it includes a linker, a scaffold, a building block, or other reactive moiety attached thereto, whereas a solid support may be “nonfunctionalized” when it lack such a reactive moiety attached thereto. The solid support can be employed free in solution, such as in a microtiter well format; in a flow-through format, such as in a column; or in a dipstick.

The solid support can comprise a membrane, paper, plastic, coated surface, flat surface, glass, slide, chip, or any combination thereof. A solid support can take the form of resins, gels, microspheres, or other geometric configurations. A solid support can comprise silica chips, microparticles, nanoparticles, plates, arrays, capillaries, flat supports such as glass fiber filters, glass surfaces, metal surfaces (steel, gold silver, aluminum, silicon and copper), glass supports, plastic supports, silicon supports, chips, filters, membranes, microwell plates, slides, plastic materials including multiwell plates or membranes (e.g., formed of polyethylene, polypropylene, polyamide, polyvinylidenedifluoride), and/or wafers, combs, pins or needles (e.g., arrays of pins suitable for combinatorial synthesis or analysis) or beads in an array of pits or nanoliter wells of flat surfaces such as wafers (e.g., silicon wafers), wafers with pits with or without filter bottoms.

The solid support can comprise a polymer matrix (e.g., gel, hydrogel). The polymer matrix may be able to permeate intracellular space (e.g., around organelles). The polymer matrix may able to be pumped throughout the circulatory system.

### Substrates and Microwell Array

As used herein, a substrate can refer to a type of solid support. A substrate can refer to a solid support that can comprise barcodes or stochastic barcodes of the disclosure. A substrate can, for example, comprise a plurality of microwells. For example, a substrate can be a well array comprising two or more microwells. In some embodiments, a microwell can comprise a small reaction chamber of defined volume. In some embodiments, a microwell can entrap one or more cells. In some embodiments, a microwell can entrap only one cell. In some embodiments, a microwell can entrap one or more solid supports. In some embodiments, a microwell can entrap only one solid support. In some embodiments, a microwell entraps a single cell and a single solid support (e.g., a bead). A microwell can comprise barcode reagents of the disclosure.

### Methods of Barcoding

The disclosure provides for methods for estimating the number of distinct targets at distinct locations in a physical sample (e.g., tissue, organ, tumor, cell). The methods can comprise placing barcodes (e.g., stochastic barcodes) in close proximity with the sample, lysing the sample, associating distinct targets with the barcodes, amplifying the targets and/or digitally counting the targets. The method can further comprise analyzing and/or visualizing the information obtained from the spatial labels on the barcodes. In some embodiments, a method comprises visualizing the plurality of targets in the sample. Mapping the plurality of targets onto the map of the sample can include generating a two dimensional map or a three dimensional map of the sample. The two dimensional map and the three dimensional map can be generated prior to or after barcoding (e.g., stochastically barcoding) the plurality of targets in the sample. Visualizing the plurality of targets in the sample can include mapping the plurality of targets onto a map of the sample. Mapping the plurality of targets onto the map of the sample can include generating a two dimensional map or a three dimensional map of the sample. The two dimensional map and the three dimensional map can be generated prior to or after barcoding the plurality of targets in the sample, in some embodiments, the two dimensional map and the three dimensional map can be generated before or after lysing the sample. Lysing the sample before or after generating the two dimensional map or the three dimensional map can include heating the sample, contacting the sample with a detergent, changing the pH of the sample, or any combination thereof.

In some embodiments, barcoding the plurality of targets comprises hybridizing a plurality of barcodes with a plurality of targets to create barcoded targets (e.g., stochastically barcoded targets). Barcoding the plurality of targets can comprise generating an indexed library of the barcoded targets. Generating an indexed library of the barcoded targets can be performed with a solid support comprising the plurality of barcodes (e.g., stochastic barcodes).

### Contacting a Sample and a Barcode

The disclosure provides for methods for contacting a sample (e.g., cells) to a substrate of the disclosure. A sample comprising, for example, a cell, organ, or tissue thin section, can be contacted to barcodes (e.g., stochastic barcodes). The cells can be contacted, for example, by gravity flow wherein the cells can settle and create a monolayer. The sample can be a tissue thin section. The thin section can be placed on the substrate. The sample can be one-dimensional (e.g., forms a planar surface). The sample (e.g., cells) can be spread across the substrate, for example, by growing/culturing the cells on the substrate.

When barcodes are in close proximity to targets, the targets can hybridize to the barcode. The barcodes can be contacted at a non-depletable ratio such that each distinct target can associate with a distinct barcode of the disclosure. To ensure efficient association between the target and the barcode, the targets can be cross-linked to barcode.

### Cell Lysis

Following the distribution of cells and barcodes, the cells can be lysed to liberate the target molecules. Cell lysis can be accomplished by any of a variety of means, for example, by chemical or biochemical means, by osmotic shock, or by means of thermal lysis, mechanical lysis, or optical lysis. Cells can be lysed by addition of a cell lysis buffer comprising a detergent (e.g., SDS, Li dodecyl sulfate, Triton X-100, Tween-20, or NP-40), an organic solvent (e.g., methanol or acetone), or digestive enzymes (e.g., proteinase K, pepsin, or trypsin), or any combination thereof. To increase the association of a target and a barcode, the rate of the diffusion of the target molecules can be altered by for example, reducing the temperature and/or increasing the viscosity of the lysate.

In some embodiments, the sample can be lysed using a filter paper. The filter paper can be soaked with a lysis buffer on top of the filter paper. The filter paper can be applied to the sample with pressure which can facilitate lysis of the sample and hybridization of the targets of the sample to the substrate.

In some embodiments, lysis can be performed by mechanical lysis, heat lysis, optical lysis, and/or chemical lysis. Chemical lysis can include the use of digestive enzymes such as proteinase K, pepsin, and trypsin. Lysis can be performed by the addition of a lysis buffer to the substrate. A lysis buffer can comprise Tris HCl. A lysis buffer can comprise at least about 0.01, 0.05, 0.1, 0.5, or 1 M or more Tris HCl. A lysis buffer can comprise at most about 0.01, 0.05, 0.1, 0.5, or 1 M or more Tris HCL. A lysis buffer can comprise about 0.1 M Tris HCl. The pH of the lysis buffer can be at least about 1, 2, 3, 4, 5, 6, 7, 8, 9, 10, or more. The pH of the lysis buffer can be at most about 1, 2, 3, 4, 5, 6, 7, 8, 9, 10, or more. In some embodiments, the pH of the lysis buffer is about 7.5. The lysis buffer can comprise a salt (e.g., LiCl). The concentration of salt in the lysis buffer can be at least about 0.1, 0.5, or 1 M or more. The concentration of salt in the lysis buffer can be at most about 0.1, 0.5, or 1 M or more. In some embodiments, the concentration of salt in the lysis buffer is about 0.5M. The lysis buffer can comprise a detergent (e.g., SDS, Li dodecyl sulfate, triton X, tween, NP-40). The concentration of the detergent in the lysis buffer can be at least about 0.0001%, 0.0005%, 0.001%, 0.005%, 0.01%, 0.05%, 0.1%, 0.5%, 1%, 2%, 3%, 4%, 5%, 6%, or 7%, or more. The concentration of the detergent in the lysis buffer can be at most about 0.0001%, 0.0005%, 0.001%, 0.005%, 0.01%, 0.05%, 0.1%, 0.5%, 1%, 2%, 3%, 4%, 5%, 6%, or 7%, or more. In some embodiments, the concentration of the detergent in the lysis buffer is about 1% Li dodecyl sulfate. The time used in the method for lysis can be dependent on the amount of detergent used. In some embodiments, the more detergent used, the less time needed for lysis. The lysis buffer can comprise a chelating agent (e.g., EDTA, EGTA). The concentration of a chelating agent in the lysis buffer can be at least about 1, 5, 10, 15, 20, 25, or 30 mM or more. The concentration of a chelating agent in the lysis buffer can be at most about 1, 5, 10, 15, 20, 25, or 30 mM or more. In some embodiments, the concentration of chelating agent in the lysis buffer is about 10 mM. The lysis buffer can comprise a reducing reagent (e.g., beta-mercaptoethanol, DTT). The concentration of the reducing reagent in the lysis buffer can be at least about 1, 5, 10, 15, or 20 mM or more. The concentration of the reducing reagent in the lysis buffer can be at most about 1, 5, 10, 15, or 20 mM or more. In some embodiments, the concentration of reducing reagent in the lysis buffer is about 5 mM. In some embodiments, a lysis buffer can comprise about 0.1M Tris HCl, about pH 7.5, about 0.5M LiCl, about 1% lithium dodecyl sulfate, about 10 mM EDTA, and about 5 mM DTT.

Lysis can be performed at a temperature of about 4, 10, 15, 20, 25, or 30° C. Lysis can be performed for about 1, 5, 10, 15, or 20 or more minutes. A lysed cell can comprise at least about 100000, 200000, 300000, 400000, 500000, 600000, or 700000 or more target nucleic acid molecules. A lysed cell can comprise at most about 100000, 200000, 300000, 400000, 500000, 600000, or 700000 or more target nucleic acid molecules.

### Attachment of Barcodes to Target Nucleic Acid Molecules

Following lysis of the cells and release of nucleic acid molecules therefrom, the nucleic acid molecules can randomly associate with the barcodes of the co-localized solid support. Association can comprise hybridization of a barcode's target recognition region to a complementary portion of the target nucleic acid molecule (e.g., oligo(dT) of the barcode can interact with a poly(A) tail of a target). The assay conditions used for hybridization (e.g., buffer pH, ionic strength, temperature, etc.) can be chosen to promote formation of specific, stable hybrids. In some embodiments, the nucleic acid molecules released from the lysed cells can associate with the plurality of probes on the substrate (e.g., hybridize with the probes on the substrate). When the probes comprise oligo(dT), mRNA molecules can hybridize to the probes and be reverse transcribed. The oligo(dT) portion of the oligonucleotide can act as a primer for first strand synthesis of the cDNA molecule. For example, in a non-limiting example of barcoding illustrated in FIG. 2, at block 216, mRNA molecules can hybridize to barcodes on beads. For example, single-stranded nucleotide fragments can hybridize to the target-binding regions of barcodes.

Attachment can further comprise ligation of a barcode's target recognition region and a portion of the target nucleic acid molecule. For example, the target binding region can comprise a nucleic acid sequence that can be capable of specific hybridization to a restriction site overhang (e.g., an EcoRI sticky-end overhang). The assay procedure can further comprise treating the target nucleic acids with a restriction enzyme (e.g., EcoRI) to create a restriction site overhang. The barcode can then be ligated to any nucleic acid molecule comprising a sequence complementary to the restriction site overhang. A ligase (e.g., T4 DNA ligase) can be used to join the two fragments.

For example, in a non-limiting example of barcoding illustrated in FIG. 2, at block 220, the labeled targets from a plurality of cells (or a plurality of samples) (e.g., target-barcode molecules) can be subsequently pooled, for example, into a tube. The labeled targets can be pooled by, for example, retrieving the barcodes and/or the beads to which the target-barcode molecules are attached.

The retrieval of solid support-based collections of attached target-barcode molecules can be implemented by use of magnetic beads and an externally-applied magnetic field. Once the target-barcode molecules have been pooled, all further processing can proceed in a single reaction vessel. Further processing can include, for example, reverse transcription reactions, amplification reactions, cleavage reactions, dissociation reactions, and/or nucleic acid extension reactions. Further processing reactions can be performed within the microwells, that is, without first pooling the labeled target nucleic acid molecules from a plurality of cells.

### Reverse Transcription or Nucleic Acid Extension

The disclosure provides for a method to create a target-barcode conjugate using reverse transcription (e.g., at block 224 of FIG. 2) or nucleic acid extension. The target-barcode conjugate can comprise the barcode and a complementary sequence of all or a portion of the target nucleic acid (i.e., a barcoded cDNA molecule, such as a stochastically barcoded cDNA molecule). Reverse transcription of the associated RNA molecule can occur by the addition of a reverse transcription primer along with the reverse transcriptase. The reverse transcription primer can be an oligo(dT) primer, a random hexanucleotide primer, or a target-specific oligonucleotide primer. Oligo (dT) primers can be, or can be about, 12-18 nucleotides in length and bind to the endogenous poly(A) tail at the 3′ end of mammalian mRNA. Random hexanucleotide primers can bind to mRNA at a variety of complementary sites. Target-specific oligonucleotide primers typically selectively prime the mRNA of interest.

In some embodiments, reverse transcription of an mRNA molecule to a labeled-RNA molecule can occur by the addition of a reverse transcription primer. In some embodiments, the reverse transcription primer is an oligo(dT) primer, random hexanucleotide primer, or a target-specific oligonucleotide primer. Generally, oligo(dT) primers are 12-18 nucleotides in length and bind to the endogenous poly(A) tail at the 3′ end of mammalian mRNA. Random hexanucleotide primers can bind to mRNA at a variety of complementary sites. Target-specific oligonucleotide primers typically selectively prime the mRNA of interest.

In some embodiments, a target is a cDNA molecule. For example, an mRNA molecule can be reverse transcribed using a reverse transcriptase, such as Moloney murine leukemia virus (MMLV) reverse transcriptase, to generate a cDNA molecule with a poly(dC) tail. A barcode can include a target binding region with a poly(dG) tail. Upon base pairing between the poly(dG) tail of the barcode and the poly(dC) tail of the cDNA molecule, the reverse transcriptase switches template strands, from cellular RNA molecule to the barcode, and continues replication to the 5′ end of the barcode. By doing so, the resulting cDNA molecule contains the sequence of the barcode (such as the molecular label) on the 3′ end of the cDNA molecule.

Reverse transcription can occur repeatedly to produce multiple labeled-cDNA molecules. The methods disclosed herein can comprise conducting at least about 1, 2, 3, 4, 5, 6, 7, 8, 9, 10, 11, 12, 13, 14, 15, 16, 17, 18, 19, or 20 reverse transcription reactions. The method can comprise conducting at least about 25, 30, 35, 40, 45, 50, 55, 60, 65, 70, 75, 80, 85, 90, 95, or 100 reverse transcription reactions.

### Amplification

One or more nucleic acid amplification reactions (e.g., at block 228 of FIG. 2) can be performed to create multiple copies of the labeled target nucleic acid molecules. Amplification can be performed in a multiplexed manner, wherein multiple target nucleic acid sequences are amplified simultaneously. The amplification reaction can be used to add sequencing adaptors to the nucleic acid molecules. The amplification reactions can comprise amplifying at least a portion of a sample label, if present. The amplification reactions can comprise amplifying at least a portion of the cellular label and/or barcode sequence (e.g., a molecular label). The amplification reactions can comprise amplifying at least a portion of a sample tag, a cell label, a spatial label, a barcode sequence (e.g., a molecular label), a target nucleic acid, or a combination thereof. The amplification reactions can comprise amplifying 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, 100%, or a range or a number between any two of these values, of the plurality of nucleic acids. The method can further comprise conducting one or more cDNA synthesis reactions to produce one or more cDNA copies of target-barcode molecules comprising a sample label, a cell label, a spatial label, and/or a barcode sequence (e.g., a molecular label).

In some embodiments, amplification can be performed using a polymerase chain reaction (PCR). As used herein, PCR can refer to a reaction for the in vitro amplification of specific DNA sequences by the simultaneous primer extension of complementary strands of DNA. As used herein, PCR can encompass derivative forms of the reaction, including but not limited to, RT-PCR, real-time PCR, nested PCR, quantitative PCR, multiplexed PCR, digital PCR, and assembly PCR.

Amplification of the labeled nucleic acids can comprise non-PCR based methods. Examples of non-PCR based methods include, but are not limited to, multiple displacement amplification (MDA), transcription-mediated amplification (TMA), nucleic acid sequence-based amplification (NASBA), strand displacement amplification (SDA), real-time SDA, rolling circle amplification, or circle-to-circle amplification. Other non-PCR-based amplification methods include multiple cycles of DNA-dependent RNA polymerase-driven RNA transcription amplification or RNA-directed DNA synthesis and transcription to amplify DNA or RNA targets, a ligase chain reaction (LCR), and a QB replicase (QB) method, use of palindromic probes, strand displacement amplification, oligonucleotide-driven amplification using a restriction endonuclease, an amplification method in which a primer is hybridized to a nucleic acid sequence and the resulting duplex is cleaved prior to the extension reaction and amplification, strand displacement amplification using a nucleic acid polymerase lacking 5′ exonuclease activity, rolling circle amplification, and ramification extension amplification (RAM). In some embodiments, the amplification does not produce circularized transcripts.

In some embodiments, the methods disclosed herein further comprise conducting a polymerase chain reaction on the labeled nucleic acid (e.g., labeled-RNA, labeled-DNA, labeled-cDNA) to produce a labeled amplicon (e.g., a stochastically labeled amplicon). The labeled amplicon can be double-stranded molecule. The double-stranded molecule can comprise a double-stranded RNA molecule, a double-stranded DNA molecule, or a RNA molecule hybridized to a DNA molecule. One or both of the strands of the double-stranded molecule can comprise a sample label, a spatial label, a cell label, and/or a barcode sequence (e.g., a molecular label). The labeled amplicon can be a single-stranded molecule. The single-stranded molecule can comprise DNA, RNA, or a combination thereof. The nucleic acids of the disclosure can comprise synthetic or altered nucleic acids.

Amplification can comprise use of one or more non-natural nucleotides. Non-natural nucleotides can comprise photolabile or triggerable nucleotides. Examples of non-natural nucleotides can include, but are not limited to, peptide nucleic acid (PNA), morpholino and locked nucleic acid (LNA), as well as glycol nucleic acid (GNA) and threose nucleic acid (TNA). Non-natural nucleotides can be added to one or more cycles of an amplification reaction. The addition of the non-natural nucleotides can be used to identify products as specific cycles or time points in the amplification reaction.

Conducting the one or more amplification reactions can comprise the use of one or more primers. The one or more primers can comprise, for example, 1, 2, 3, 4, 5, 6, 7, 8, 9, 10, 11, 12, 13, 14, or 15 or more nucleotides. The one or more primers can comprise at least 1, 2, 3, 4, 5, 6, 7, 8, 9, 10, 11, 12, 13, 14, or 15 or more nucleotides. The one or more primers can comprise less than 12-15 nucleotides. The one or more primers can anneal to at least a portion of the plurality of labeled targets (e.g., stochastically labeled targets). The one or more primers can anneal to the 3′ end or 5′ end of the plurality of labeled targets. The one or more primers can anneal to an internal region of the plurality of labeled targets. The internal region can be at least about 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ ends the plurality of labeled targets. The one or more primers can comprise a fixed panel of primers. The one or more primers can comprise at least one or more custom primers. The one or more primers can comprise at least one or more control primers. The one or more primers can comprise at least one or more gene-specific primers.

The one or more primers can comprise a universal primer. The universal primer can anneal to a universal primer binding site. The one or more custom primers can anneal to a first sample label, a second sample label, a spatial label, a cell label, a barcode sequence (e.g., a molecular label), a target, or any combination thereof. The one or more primers can comprise a universal primer and a custom primer. The custom primer can be designed to amplify one or more targets. The targets can comprise a subset of the total nucleic acids in one or more samples. The targets can comprise a subset of the total labeled targets in one or more samples. The one or more primers can comprise at least 96 or more custom primers. The one or more primers can comprise at least 960 or more custom primers. The one or more primers can comprise at least 9600 or more custom primers. The one or more custom primers can anneal to two or more different labeled nucleic acids. The two or more different labeled nucleic acids can correspond to one or more genes.

Any amplification scheme can be used in the methods of the present disclosure. For example, in one scheme, the first round PCR can amplify molecules attached to the bead using a gene specific primer and a primer against the universal Illumina sequencing primer 1 sequence. The second round of PCR can amplify the first PCR products using a nested gene specific primer flanked by Illumina sequencing primer 2 sequence, and a primer against the universal Illumina sequencing primer 1 sequence. The third round of PCR adds P5 and P7 and sample index to turn PCR products into an Illumina sequencing library. Sequencing using 150 bp×2 sequencing can reveal the cell label and barcode sequence (e.g., molecular label) on read 1, the gene on read 2, and the sample index on index 1 read.

In some embodiments, nucleic acids can be removed from the substrate using chemical cleavage. For example, a chemical group or a modified base present in a nucleic acid can be used to facilitate its removal from a solid support. For example, an enzyme can be used to remove a nucleic acid from a substrate. For example, a nucleic acid can be removed from a substrate through a restriction endonuclease digestion. For example, treatment of a nucleic acid containing a dUTP or ddUTP with uracil-d-glycosylase (UDG) can be used to remove a nucleic acid from a substrate. For example, a nucleic acid can be removed from a substrate using an enzyme that performs nucleotide excision, such as a base excision repair enzyme, such as an apurinic/apyrimidinic (AP) endonuclease. In some embodiments, a nucleic acid can be removed from a substrate using a photocleavable group and light. In some embodiments, a cleavable linker can be used to remove a nucleic acid from the substrate. For example, the cleavable linker can comprise at least one of biotin/avidin, biotin/streptavidin, biotin/neutravidin, Ig-protein A, a photo-labile linker, acid or base labile linker group, or an aptamer.

When the probes are gene-specific, the molecules can hybridize to the probes and be reverse transcribed and/or amplified. In some embodiments, after the nucleic acid has been synthesized (e.g., reverse transcribed), it can be amplified. Amplification can be performed in a multiplex manner, wherein multiple target nucleic acid sequences are amplified simultaneously. Amplification can add sequencing adaptors to the nucleic acid.

In some embodiments, amplification can be performed on the substrate, for example, with bridge amplification. cDNAs can be homopolymer tailed in order to generate a compatible end for bridge amplification using oligo(dT) probes on the substrate. In bridge amplification, the primer that is complementary to the 3′ end of the template nucleic acid can be the first primer of each pair that is covalently attached to the solid particle. When a sample containing the template nucleic acid is contacted with the particle and a single thermal cycle is performed, the template molecule can be annealed to the first primer and the first primer is elongated in the forward direction by addition of nucleotides to form a duplex molecule consisting of the template molecule and a newly formed DNA strand that is complementary to the template. In the heating step of the next cycle, the duplex molecule can be denatured, releasing the template molecule from the particle and leaving the complementary DNA strand attached to the particle through the first primer. In the annealing stage of the annealing and elongation step that follows, the complementary strand can hybridize to the second primer, which is complementary to a segment of the complementary strand at a location removed from the first primer. This hybridization can cause the complementary strand to form a bridge between the first and second primers secured to the first primer by a covalent bond and to the second primer by hybridization. In the elongation stage, the second primer can be elongated in the reverse direction by the addition of nucleotides in the same reaction mixture, thereby converting the bridge to a double-stranded bridge. The next cycle then begins, and the double-stranded bridge can be denatured to yield two single-stranded nucleic acid molecules, each having one end attached to the particle surface via the first and second primers, respectively, with the other end of each unattached. In the annealing and elongation step of this second cycle, each strand can hybridize to a further complementary primer, previously unused, on the same particle, to form new single-strand bridges. The two previously unused primers that are now hybridized elongate to convert the two new bridges to double-strand bridges.

The amplification reactions can comprise amplifying at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 97%, or 100% of the plurality of nucleic acids.

Amplification of the labeled nucleic acids can comprise PCR-based methods or non-PCR based methods. Amplification of the labeled nucleic acids can comprise exponential amplification of the labeled nucleic acids. Amplification of the labeled nucleic acids can comprise linear amplification of the labeled nucleic acids. Amplification can be performed by polymerase chain reaction (PCR). PCR can refer to a reaction for the in vitro amplification of specific DNA sequences by the simultaneous primer extension of complementary strands of DNA. PCR can encompass derivative forms of the reaction, including but not limited to, RT-PCR, real-time PCR, nested PCR, quantitative PCR, multiplexed PCR, digital PCR, suppression PCR, semi-suppressive PCR and assembly PCR.

Amplification of the labeled nucleic acids can comprise non-PCR based methods, including, but not limited to, MDA, TMA, NASBA, SDA, real-time SDA, rolling circle amplification, circle-to-circle amplification, multiple cycles of DNA-dependent RNA polymerase-driven RNA transcription amplification or RNA-directed DNA synthesis and transcription to amplify DNA or RNA targets, a ligase chain reaction (LCR), a QB replicase (QB), use of palindromic probes, strand displacement amplification, oligonucleotide-driven amplification using a restriction endonuclease, an amplification method in which a primer is hybridized to a nucleic acid sequence and the resulting duplex is cleaved prior to the extension reaction and amplification, strand displacement amplification using a nucleic acid polymerase lacking 5′ exonuclease activity, rolling circle amplification, and/or RAM.

In some embodiments, the methods disclosed herein further comprise conducting a nested polymerase chain reaction on the amplified amplicon (e.g., target). The amplicon can be double-stranded molecule. The double-stranded molecule can comprise a double-stranded RNA molecule, a double-stranded DNA molecule, or a RNA molecule hybridized to a DNA molecule. One or both of the strands of the double-stranded molecule can comprise a sample tag or molecular identifier label. Alternatively, the amplicon can be a single-stranded molecule. The single-stranded molecule can comprise DNA, RNA, or a combination thereof. The nucleic acids of the present invention can comprise synthetic or altered nucleic acids.

In some embodiments, the method comprises repeatedly amplifying the labeled nucleic acid to produce multiple amplicons. The methods disclosed herein can comprise conducting at least about 1, 2, 3, 4, 5, 6, 7, 8, 9, 10, 11, 12, 13, 14, 15, 16, 17, 18, 19, or 20 amplification reactions. Alternatively, the method comprises conducting at least about 25, 30, 35, 40, 45, 50, 55, 60, 65, 70, 75, 80, 85, 90, 95, or 100 amplification reactions.

Amplification can further comprise adding one or more control nucleic acids to one or more samples comprising a plurality of nucleic acids. Amplification can further comprise adding one or more control nucleic acids to a plurality of nucleic acids. The control nucleic acids can comprise a control label.

Amplification can comprise use of one or more non-natural nucleotides. Non-natural nucleotides can comprise photolabile and/or triggerable nucleotides. Examples of non-natural nucleotides include, but are not limited to, peptide nucleic acid (PNA), morpholino and locked nucleic acid (LNA), as well as glycol nucleic acid (GNA) and threose nucleic acid (TNA). Non-natural nucleotides can be added to one or more cycles of an amplification reaction. The addition of the non-natural nucleotides can be used to identify products as specific cycles or time points in the amplification reaction.

Conducting the one or more amplification reactions can comprise the use of one or more primers. The one or more primers can comprise one or more oligonucleotides. The one or more oligonucleotides can comprise at least about 7-9 nucleotides. The one or more oligonucleotides can comprise less than 12-15 nucleotides. The one or more primers can anneal to at least a portion of the plurality of labeled nucleic acids. The one or more primers can anneal to the 3′ end and/or 5′ end of the plurality of labeled nucleic acids. The one or more primers can anneal to an internal region of the plurality of labeled nucleic acids. The internal region can be at least about 50, 100, 150, 200, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 650, 700, 750, 800, 850, 900 or 1000 nucleotides from the 3′ ends the plurality of labeled nucleic acids. The one or more primers can comprise a fixed panel of primers. The one or more primers can comprise at least one or more custom primers. The one or more primers can comprise at least one or more control primers. The one or more primers can comprise at least one or more housekeeping gene primers. The one or more primers can comprise a universal primer. The universal primer can anneal to a universal primer binding site. The one or more custom primers can anneal to the first sample tag, the second sample tag, the molecular identifier label, the nucleic acid or a product thereof. The one or more primers can comprise a universal primer and a custom primer. The custom primer can be designed to amplify one or more target nucleic acids. The target nucleic acids can comprise a subset of the total nucleic acids in one or more samples. In some embodiments, the primers are the probes attached to the array of the disclosure.

In some embodiments, barcoding (e.g., stochastically barcoding) the plurality of targets in the sample further comprises generating an indexed library of the barcoded targets (e.g., stochastically barcoded targets) or barcoded fragments of the targets. The barcode sequences of different barcodes (e.g., the molecular labels of different stochastic barcodes) can be different from one another. Generating an indexed library of the barcoded targets includes generating a plurality of indexed polynucleotides from the plurality of targets in the sample. For example, for an indexed library of the barcoded targets comprising a first indexed target and a second indexed target, the label region of the first indexed polynucleotide can differ from the label region of the second indexed polynucleotide by, by about, by at least, or by at most, 1, 2, 3, 4, 5, 6, 7, 8, 9, 10, 20, 30, 40, 50, or a number or a range between any two of these values, nucleotides. In some embodiments, generating an indexed library of the barcoded targets includes contacting a plurality of targets, for example mRNA molecules, with a plurality of oligonucleotides including a poly(T) region and a label region; and conducting a first strand synthesis using a reverse transcriptase to produce single-strand labeled cDNA molecules each comprising a cDNA region and a label region, wherein the plurality of targets includes at least two mRNA molecules of different sequences and the plurality of oligonucleotides includes at least two oligonucleotides of different sequences. Generating an indexed library of the barcoded targets can further comprise amplifying the single-strand labeled cDNA molecules to produce double-strand labeled cDNA molecules; and conducting nested PCR on the double-strand labeled cDNA molecules to produce labeled amplicons. In some embodiments, the method can include generating an adaptor-labeled amplicon.

Barcoding (e.g., stochastic barcoding) can include using nucleic acid barcodes or tags to label individual nucleic acid (e.g., DNA or RNA) molecules. In some embodiments, it involves adding DNA barcodes or tags to cDNA molecules as they are generated from mRNA. Nested PCR can be performed to minimize PCR amplification bias. Adaptors can be added for sequencing using, for example, next generation sequencing (NGS). The sequencing results can be used to determine cell labels, molecular labels, and sequences of nucleotide fragments of the one or more copies of the targets, for example at block 232 of FIG. 2.

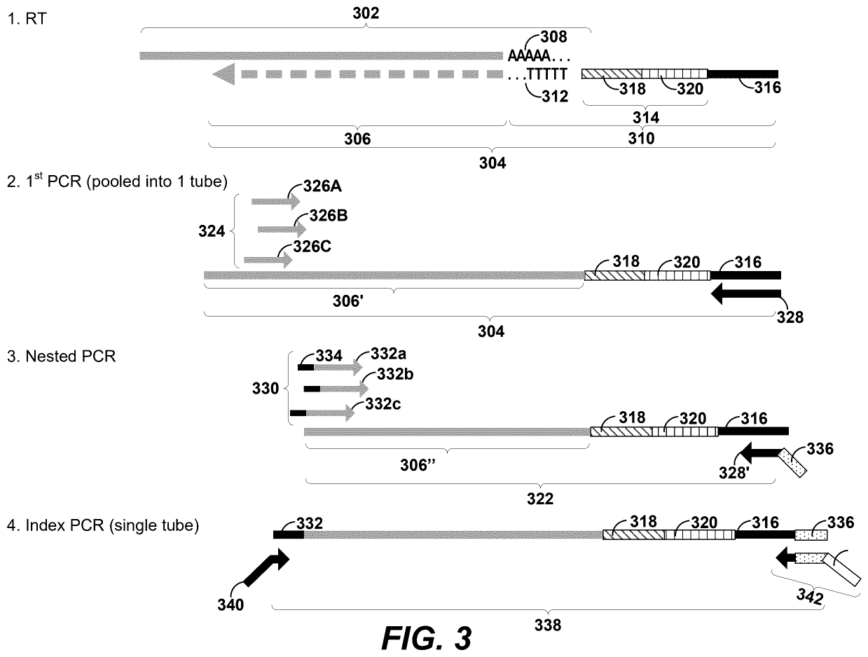

FIG. 3 is a schematic illustration showing a non-limiting exemplary process of generating an indexed library of the barcoded targets (e.g., stochastically barcoded targets), such as barcoded mRNAs or fragments thereof. As shown in step 1, the reverse transcription process can encode each mRNA molecule with a unique molecular label sequence, a cell label sequence, and a universal PCR site. In particular, RNA molecules 302 can be reverse transcribed to produce labeled cDNA molecules 304, including a cDNA region 306, by hybridization (e.g., stochastic hybridization) of a set of barcodes (e.g., stochastic barcodes) 310 to the poly(A) sitail region 308 of the RNA molecules 302. Each of the barcodes 310 can comprise a target-binding region, for example a poly(dT) region 312, a label region 314 (e.g., a barcode sequence or a molecule), and a universal PCR region 316.

In some embodiments, the cell label sequence can include 3 to 20 nucleotides. In some embodiments, the molecular label sequence can include 3 to 20 nucleotides. In some embodiments, each of the plurality of stochastic barcodes further comprises one or more of a universal label and a cell label, wherein universal labels are the same for the plurality of stochastic barcodes on the solid support and cell labels are the same for the plurality of stochastic barcodes on the solid support. In some embodiments, the universal label can include 3 to 20 nucleotides. In some embodiments, the cell label comprises 3 to 20 nucleotides.

In some embodiments, the label region 314 can include a barcode sequence or a molecular label 318 and a cell label 320. In some embodiments, the label region 314 can include one or more of a universal label, a dimension label, and a cell label. The barcode sequence or molecular label 318 can be, can be about, can be at least, or can be at most, 1, 2, 3, 4, 5, 6, 7, 8, 9, 10, 20, 30, 40, 50, 60, 70, 80, 90, 100, or a number or a range between any of these values, of nucleotides in length. The cell label 320 can be, can be about, can be at least, or can be at most, 1, 2, 3, 4, 5, 6, 7, 8, 9, 10, 20, 30, 40, 50, 60, 70, 80, 90, 100, or a number or a range between any of these values, of nucleotides in length. The universal label can be, can be about, can be at least, or can be at most, 1, 2, 3, 4, 5, 6, 7, 8, 9, 10, 20, 30, 40, 50, 60, 70, 80, 90, 100, or a number or a range between any of these values, of nucleotides in length. Universal labels can be the same for the plurality of stochastic barcodes on the solid support and cell labels are the same for the plurality of stochastic barcodes on the solid support. The dimension label can be, can be about, can be at least, or can be at most 1, 2, 3, 4, 5, 6, 7, 8, 9, 10, 20, 30, 40, 50, 60, 70, 80, 90, 100, or a number or a range between any of these values, of nucleotides in length.

In some embodiments, the label region 314 can comprise, comprise about, comprise at least, or comprise at most, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any of these values, different labels, such as a barcode sequence or a molecular label 318 and a cell label 320. Each label can be, can be about, can be at least, or can be at most 1, 2, 3, 4, 5, 6, 7, 8, 9, 10, 20, 30, 40, 50, 60, 70, 80, 90, 100, or a number or a range between any of these values, of nucleotides in length. A set of barcodes or stochastic barcodes 310 can contain, contain about, contain at least, or can be at most, 10, 20, 40, 50, 70, 80, 90, 10², 10³, 10⁴, 10⁵, 10⁶, 10⁷, 10⁸, 10⁹, 10¹⁰, 10¹¹, 10¹², 10¹³, 10¹⁴, 10¹⁵, 10²⁰, or a number or a range between any of these values, barcodes or stochastic barcodes 310. And the set of barcodes or stochastic barcodes 310 can, for example, each contain a unique label region 314. The labeled cDNA molecules 304 can be purified to remove excess barcodes or stochastic barcodes 310. Purification can comprise Ampure bead purification.

As shown in step 2, products from the reverse transcription process in step 1 can be pooled into 1 tube and PCR amplified with a 1^{st} PCR primer pool and a 1^{st} universal PCR primer. Pooling is possible because of the unique label region 314. In particular, the labeled cDNA molecules 304 can be amplified to produce nested PCR labeled amplicons 322.

Amplification can comprise multiplex PCR amplification. Amplification can comprise a multiplex PCR amplification with 96 multiplex primers in a single reaction volume. In some embodiments, multiplex PCR amplification can utilize, utilize about, utilize at least, or utilize at most, 10, 20, 40, 50, 70, 80, 90, 10², 10³, 10⁴, 10⁵, 10⁶, 10⁷, 10⁸, 10⁹, 10¹⁰, 10¹¹, 10¹², 10¹³, 10¹⁴, 10¹⁵, 10²⁰, or a number or a range between any of these values, multiplex primers in a single reaction volume. Amplification can comprise using a 1^{st} PCR primer pool 324 comprising custom primers 326A-C targeting specific genes and a universal primer 328. The custom primers 326 can hybridize to a region within the cDNA portion 306′ of the labeled cDNA molecule 304. The universal primer 328 can hybridize to the universal PCR region 316 of the labeled cDNA molecule 304.

As shown in step 3 of FIG. 3, products from PCR amplification in step 2 can be amplified with a nested PCR primers pool and a 2^{nd} universal PCR primer. Nested PCR can minimize PCR amplification bias. In particular, the nested PCR labeled amplicons 322 can be further amplified by nested PCR. The nested PCR can comprise multiplex PCR with nested PCR primers pool 330 of nested PCR primers 332a-c and a 2^{nd} universal PCR primer 328′ in a single reaction volume. The nested PCR primer pool 328 can contain, contain about, contain at least, or contain at most, 1, 2, 3, 4, 5, 6, 7, 8, 9, 10, 20, 30, 40, 50, 60, 70, 80, 90, 100, 200, 300, 400, 500, 600, 700, 800, 900, 1000, or a number or a range between any of these values, different nested PCR primers 330. The nested PCR primers 332 can contain an adaptor 334 and hybridize to a region within the cDNA portion 306″ of the labeled amplicon 322. The universal primer 328′ can contain an adaptor 336 and hybridize to the universal PCR region 316 of the labeled amplicon 322. Thus, step 3 produces adaptor-labeled amplicon 338. In some embodiments, nested PCR primers 332 and the 2^{nd} universal PCR primer 328′ may not contain the adaptors 334 and 336. The adaptors 334 and 336 can instead be ligated to the products of nested PCR to produce adaptor-labeled amplicon 338.

As shown in step 4, PCR products from step 3 can be PCR amplified for sequencing using library amplification primers. In particular, the adaptors 334 and 336 can be used to conduct one or more additional assays on the adaptor-labeled amplicon 338. The adaptors 334 and 336 can be hybridized to primers 340 and 342. The one or more primers 340 and 342 can be PCR amplification primers. The one or more primers 340 and 342 can be sequencing primers. The one or more adaptors 334 and 336 can be used for further amplification of the adaptor-labeled amplicons 338. The one or more adaptors 334 and 336 can be used for sequencing the adaptor-labeled amplicon 338. The primer 342 can contain a plate index 344 so that amplicons generated using the same set of barcodes or stochastic barcodes 310 can be sequenced in one sequencing reaction using next generation sequencing (NGS).

### Chambered Single Cell Barcoding

High throughput screening can be done through sample multiplexing kit, where different cell populations can be stained with an antibody containing a sample barcoded oligo. However, in some embodiments, these additional staining steps are not preferred as they may change the biological response. Provided herein include compositions, systems, and methods for chambered Rhapsody with unique barcodes (e.g., chamber indexing subsequence(s)). There are provided, compositions and methods for sorting into barcoded chambers on Rhapsody cartridge for single cell workflows. In order to correlate image or fluorescence based sorting with single cell data, it can be important to have a means of sorting cells into chambers such that the information is retained as to which population of cells (based on gating) is sorted into which chambers. Provided herein include compositions and methods comprising a chambered Rhapsody cartridge, where each chamber can be loaded with beads containing unique set of cell labels.

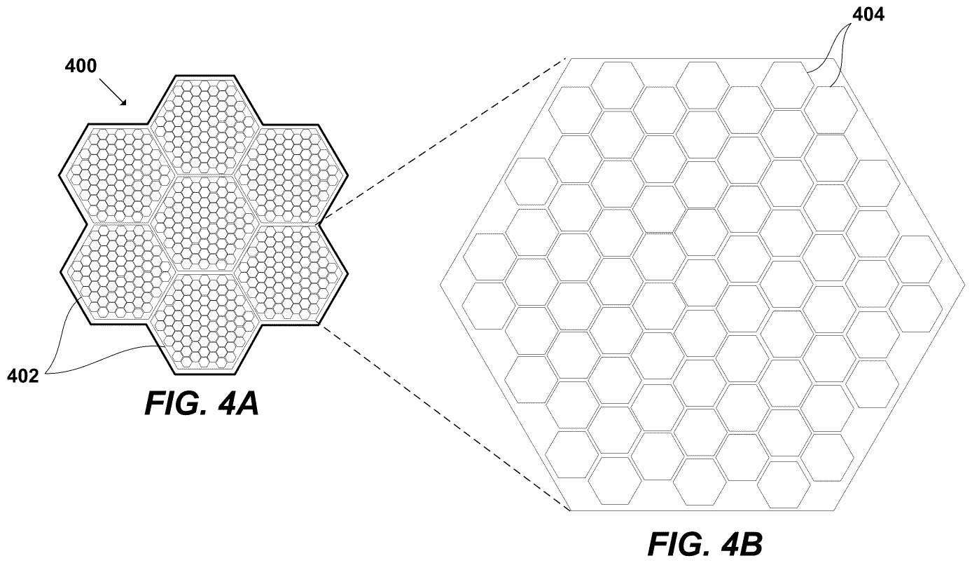

There are provided, in some embodiments, scheme for solid support (e.g., bead) manufacturing. There are provided, in some embodiments, x number of unique cell label sets, where each set contains unique number of y cell labels. For example, a 384-well chambered cartridge can comprise 384 unique sets of cell labels. There can be several thousand microwells within each of these chambers. Cell sorters can directly sort different cell populations into these chambers (e.g., as they would into 384 well plates). There are provided, in some embodiments, workflows to maintain cell viability in the cartridge and the rest of the single cell workflows (e.g., Rhapsody) provided herein. Some embodiments of the compositions and methods provided herein comprise automated liquid handling. FIGS. 4A-4B depict non-limiting exemplary schematics of a microwell array (FIG. 4A) and chamber (FIG. 4B) provided herein. FIG. 4A depicts a non-limiting exemplary schematic of a microwell array 400 comprising a plurality of chambers 402. Each of the chambers can comprise a plurality of partitions 404 (e.g., microwells).

There are provided, in some embodiments, methods and compositions enabling the differentiation of library samples after sequencing with cell barcodes, which can simplify user workflow(s). Disclosed herein include methods and compositions employing cell barcodes for sample and lane identification. In some embodiments, the methods comprise assigning specific cell barcodes (e.g., comprising chamber-specific chamber indexing subsequence(s)) to each chamber of a multi-chamber cartridge (e.g., HT Xpress System 8-Lane Cartridge) while maintaining high cell barcode diversity by manufacturing, e.g., 8 different bead lots. By assigning specific cell labels to each lane (e.g., chamber), users can identify their library samples when performing multi-lane experiments without the need for assigning specific forward and reverse primers to each lane and library type. This can enable an efficient workflow for users to prepare their libraries for sequencing.

Disclosed herein are pluralities of solid supports comprising chamber-specific chamber indexing subsequence(s) (e.g., 8 different barcoded beads for each HT lane), such that users can differentiate between the chambers (e.g., lanes) rather than having to use library primers with indices (e.g., 8 different reverse primers). Current methods require users to differentiate their library samples with the use of different forward and reverse primers. The methods and compositions provided herein can eliminate the need to use multiple different forward and reverse primers by enabling users to differentiate based on cell barcodes.

In some embodiments, cell label diversity is 384*384*384=56623104 different cell barcode combinations, where there are 384 Cell Label 1 (first cell label portion; CL1), 384 Cell Label 2 (second cell label portion; CL2) and 384 Cell Label 3 (third cell label portion; CL3). It has been simulated that at most 192*192*192=7077888 different cell barcode combinations are needed to maintain less than 1% cell barcode collision. In some embodiments, methods and compositions provided herein use the same 384 cell label portions across three different oligos but distributed in a way such that each bead lot has 7077888 different cell barcodes. Methods provided herein can generate 8 different bead lots with specific cell barcodes. Upfront users can assign specific bead lots to each lane. For 8 lanes, for example, users can use 8 different bead lots. Currently users assign different reverse primers to differentiate their lanes. For example, if a user were to do Whole Transcriptome Analysis (WTA) for 8 lanes with 8 different samples, instead of keeping track of 8 different reverse primers, using the provided compositions and methods as long as they input their beads into their respective lane, in the end they would only need to use 1 reverse primer and bioinformatically could tell their samples apart. For example, Table 1 illustrates the current method of using library indices on primers to distinguish the lane of origin (for Targeted or WTA Library Amplification).

TABLE 1
Current Library Indices Method

| Lane | Forward Primer | Reverse Primer |
|---|---|---|
| 1 | D50X | D701 |
| 2 | D50X | D702 |
| 3 | D50X | D703 |
| 4 | D50X | D704 |
| 5 | D50X | D705 |
| 6 | D50X | D706 |
| 7 | D50X | D707 |
| 8 | D50X | D708 |

Furthermore, with increasing library combination complexity, the number of forward and reverse indices to keep track of increases using the current methodology. Thus, for example, for 8 lanes for WTA only, 8 library indices are employed, but for 8 lanes for WTA+AbSeq, this increases to 16 library indices, and for 8 lanes for WTA+AbSeq+ST (e.g., sample tagging), this increases to 24 library indices. In contrast, Table 2 illustrates an exemplary setup using the compositions and methods provided herein—with different cell barcodes assigned per lane, users can only need one forward/reverse primer to keep track of for each library type.

TABLE 2
Improved Method Wherein Cell Barcodes Assigned Per Lane

| Lane | Forward Primer | Reverse Primer |
|---|---|---|
| 1 | D50X | D701 |
| 2 | | |
| 3 | | |
| 4 | | |
| 5 | | |
| 6 | | |
| 7 | | |
| 8 | | |

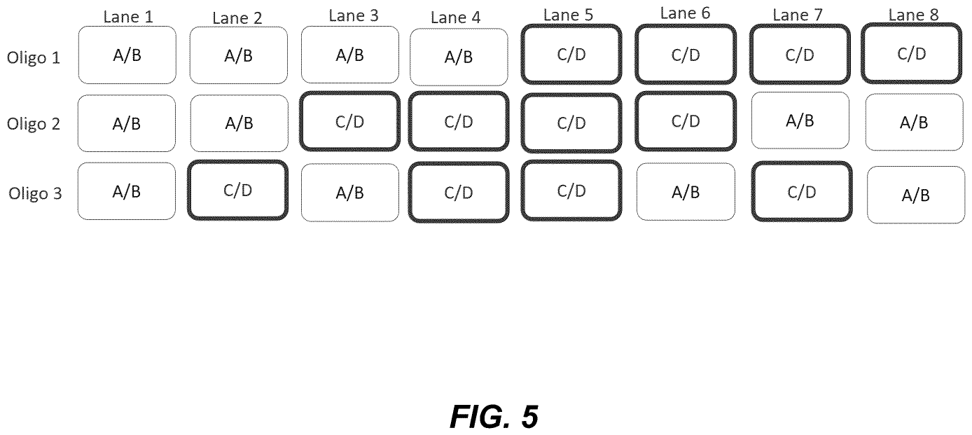

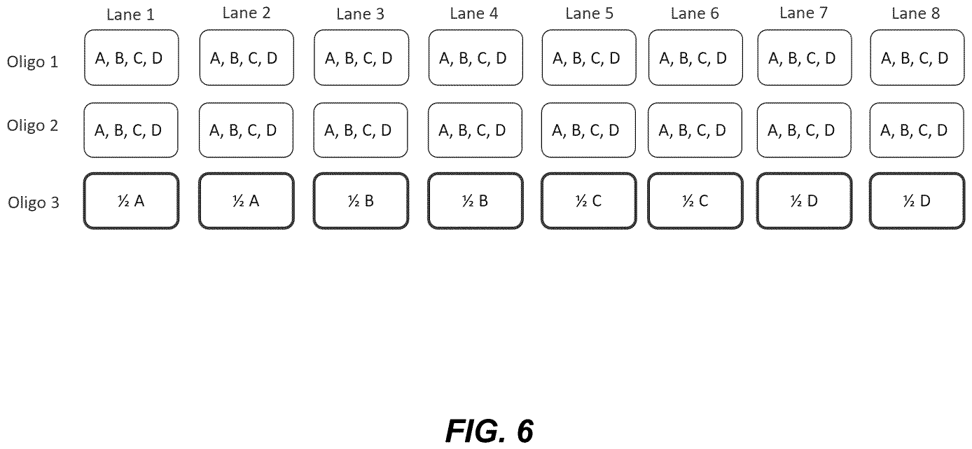

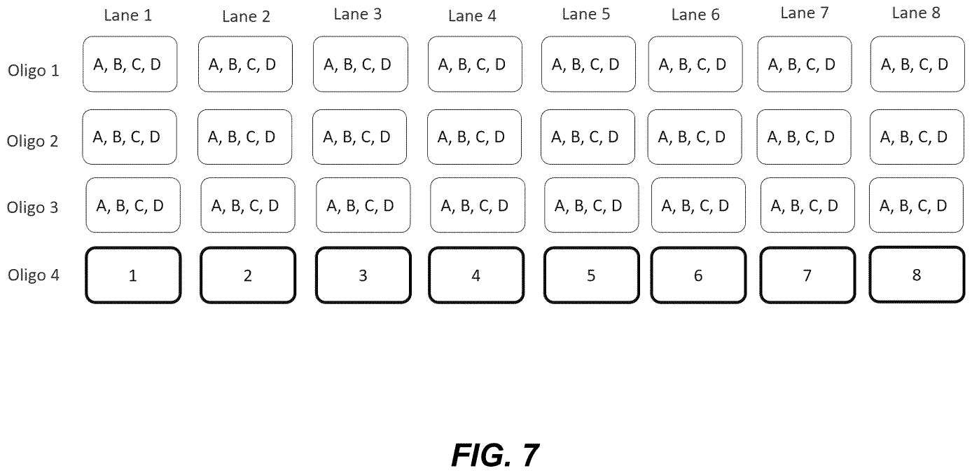

Provided herein include methods and compositions for making and using the oligonucleotide barcodes comprising chamber indexing subsequence(s) (e.g., solid supports comprising chamber-specific chamber indexing subsequence(s)) provided herein. FIG. 5 depicts a non-limiting exemplary method for manufacturing oligonucleotide barcodes comprising chamber indexing subsequence(s) provided herein. Each block represents 2*96-well plates (for total of 192 CL). The manufacturing of eight pluralities of solid supports (which can be employed by a user to distinguish 8 lanes/chambers) is depicted. This method can use (192 CL1)*(192 CL2)*(192 CL3) to generate 7077888 different combinations. The letters A, B, C, and D represent different populations of oligonucleotides with different cell label portion sequences. FIG. 6 depicts another non-limiting exemplary method for manufacturing oligonucleotide barcodes comprising chamber indexing subsequence(s) provided herein. A user can, for example, combine 384 CL1, 384 CL2, and 48 different CL3 for each lane. This method can simplifies bead manufacturing, and can maintain 7077888 cell barcode diversity (384*384*48). This manufacturing method can be similar to the current process, but pool only half plates at the Oligo 3 step. FIG. 7 depicts another non-limiting exemplary method for manufacturing oligonucleotide barcodes comprising chamber indexing subsequence(s) provided herein. This manufacturing method adds bulk ligation at the end of the current process to add a differentiating cell label with Oligo 4. Cell barcode diversity can be 384*384*384*1=~56 million. In some embodiments, Oligo 4 is configured to mitigate the increased sequencing read length, which can lower library diversity for RI in some embodiments. The sequences of cell label portions can be the same between first, second, and third oligonucleotides. For example, in some embodiments, a first oligonucleotide, and second oligonucleotide, and/or a third oligonucleotide can have first cell label portion, a second cell label portion, or a third cell label portion, respectively, comprising the sequence of AAAGG.

Provided herein are two or more pluralities of solid supports (e.g., first solid supports, second solid supports, third solid supports, etc.) wherein each plurality of solid supports has unique chamber specific chamber indexing subsequence(s). Each plurality of solid supports can be introduced into distinct chambers (e.g., first solid supports placed in lane 1, second solid supports placed in lane 2). Based on the chamber specific chamber indexing subsequence(s) unique to each plurality of solid supports, from the sequencing read it can be ascertained if it is a product of an oligonucleotide barcode associated with, e.g., a first solid support or a second solid support (and thereby ascertain if it is derived from lane 1 or lane 2). For example, first solid supports of a plurality of first solid supports can have first chamber indexing subsequence(s) that enables a user to uniquely identify from a sequencing read that is originated from a first solid support. In some embodiments, the first chamber indexing subsequence(s) is a single subsequence of the cell label. For example, referring to FIG. 7, in some embodiments the first chamber indexing subsequence(s) is a single cell label portion (such as the fourth cell label portion, as shown in FIG. 7), and the unique subsequence is the same across all first solid supports (e.g., is not selected from a set of chamber indexing subsequence(s)). In this case, only the fourth cell label portion of the cell label is employed to distinguish from solid supports (and thereby chamber). In some embodiments, such as shown in FIG. 6, a single subsequence of the cell label is employed to distinguish from solid supports (and thereby chamber), but the chamber indexing subsequence(s) are selected from a set of chamber indexing subsequence(s). For oligonucleotide barcodes derived from the method of FIG. 6, the chamber indexing subsequence can be the third cell label portion. If, for example, 48 different oligo 3 (comprising CL3; e.g., a first half of population A third oligonucleotides) are used for generation of first solid supports then the first chamber indexing subsequence has one of the sequences of a set of 48 chamber indexing subsequences. Second solid supports can have a CL3 derived from the second half of population A third oligonucleotides. Thus, second solid supports can have a second chamber indexing subsequence which has one of the sequences of a non-overlapping set of another 48 chamber indexing subsequences (the second half of population A of third oligonucleotides). Since the third cell label portion of first solid supports and second solid supports are derived from non-overlapping subsets of the third cell label portion sequences, a user can, based on the sequence of the third label portion, determine if the sequencing read derived from a first solid support or a second solid support. The chamber indexing subsequence is thus predetermined as a user knows, for both first solid supports and second solid supports, what the set of possible sequences chamber indexing subsequences is for each one.

In some embodiments, predetermined chamber indexing subsequence(s) can comprise multiple (or all) components of the cell label. For example, using the process shown in FIG. 5 eight pluralities of solid supports can be generated (to distinguish different chambers/lanes). Each plurality of solid supports can be derived from different combinations of populations of first oligonucleotides, second oligonucleotides, and third oligonucleotides. For example, with regards to the first solid supports (for use in chamber/lane 1), these can be derived from a first population of first oligonucleotides, a first population of second oligonucleotides, and a first population of third oligonucleotides, while second solid supports (for use in chamber/lane 2) can be derived from a second population of first oligonucleotides, a second population of second oligonucleotides, and a second population of third oligonucleotides. In this case, the first and second populations of first oligonucleotides are the same (A/B), the first and second populations of second oligonucleotides are the same (A/B), and the first and second populations of third oligonucleotides are different (A/B versus C/D). Since the CL3 sequence of the first and second populations of third oligonucleotides are different from each other, first solid support and second solid supports can be distinguished from each other on the basis of the third cell label portion (chamber indexing subsequence). All eight lanes differ from each other with respect to at least one of oligo 1, oligo 2, or oligo 3, and therefore can be distinguished on the basis of one, two, or three, chamber indexing subsequences.

The composition disclosed herein can comprise two or more pluralities of solid supports. The solid supports can each comprise a plurality of oligonucleotide barcodes each comprising a cell label sequence, wherein each cell label sequence comprises predetermined chamber indexing subsequence(s). In some embodiments, oligonucleotide barcodes associated with the same solid support comprise the same cell label sequence, wherein oligonucleotide barcodes associated with different solid supports comprise different cell label sequences.

In some embodiments, the two or more pluralities of solid supports comprise a plurality of first solid supports and a plurality of second solid supports, wherein oligonucleotide barcodes associated with plurality of first solid supports have a first predetermined chamber indexing subsequence(s), wherein oligonucleotide barcodes associated with plurality of second solid supports have a second predetermined chamber indexing subsequence(s), and wherein the first predetermined chamber indexing subsequence(s) and the second predetermined chamber indexing subsequence(s) are different.

In some embodiments, the two or more pluralities of solid supports comprise a plurality of first solid supports and a plurality of second solid supports, wherein the first predetermined chamber indexing subsequence(s) of the plurality of first solid supports are selected from a first set of chamber indexing subsequence(s), wherein the second predetermined chamber indexing subsequence(s) of the plurality of second solid supports are selected from a second set of chamber indexing subsequence(s), and wherein each of the chamber indexing subsequence(s) of first set of chamber indexing subsequence(s) differ from the chamber indexing subsequence(s) of the second set of chamber indexing subsequence(s). In some embodiments, based on the predetermined chamber indexing subsequence(s) of a sequencing read derived from an oligonucleotide barcode, or a product thereof, a user can determine if the oligonucleotide barcode is associated with the plurality of first solid supports or the plurality of second solid supports.

The two or more pluralities of solid supports can comprise: a plurality of third solid supports, optionally: (a) oligonucleotide barcodes associated with the plurality of third solid supports have a third chamber indexing subsequence(s) and/or (b) third chamber indexing subsequence(s) of the plurality of third solid supports are selected from a third set of chamber indexing subsequence(s); a plurality of fourth solid supports, optionally: (a) oligonucleotide barcodes associated with the plurality of fourth solid supports have a fourth chamber indexing subsequence(s) and/or (b) fourth chamber indexing subsequence(s) of the plurality of fourth solid supports are selected from a fourth set of chamber indexing subsequence(s); a plurality of fifth solid supports, optionally: (a) oligonucleotide barcodes associated with the plurality of fifth solid supports have a fifth chamber indexing subsequence(s) and/or (b) fifth chamber indexing subsequence(s) of the plurality of fifth solid supports are selected from a fifth set of chamber indexing subsequence(s); a plurality of sixth solid supports, optionally: (a) oligonucleotide barcodes associated with the plurality of sixth solid supports have a sixth chamber indexing subsequence(s) and/or (b) sixth chamber indexing subsequence(s) of the plurality of sixth solid supports are selected from a sixth set of chamber indexing subsequence(s); a plurality of seventh solid supports, optionally: (a) oligonucleotide barcodes associated with the plurality of seventh solid supports have a seventh chamber indexing subsequence(s) and/or (b) seventh chamber indexing subsequence(s) of the plurality of seventh solid supports are selected from a seventh set of chamber indexing subsequence(s); and/or a plurality of eighth solid supports, optionally: (a) oligonucleotide barcodes associated with the plurality of eighth solid supports have a eighth chamber indexing subsequence(s) and/or (b) eighth chamber indexing subsequence(s) of the plurality of eighth solid supports are selected from an eighth set of chamber indexing subsequence(s). In some embodiments, the first chamber indexing subsequence(s), the second chamber indexing subsequence(s), the third chamber indexing subsequence(s), the fourth chamber indexing subsequence(s), the fifth chamber indexing subsequence(s), the sixth chamber indexing subsequence(s), the seventh chamber indexing subsequence(s), and/or the eighth chamber indexing subsequence(s), do not share a sequence with each other. In some embodiments, based on the predetermined chamber indexing subsequence(s) of a sequencing read derived from an oligonucleotide barcode, or a product thereof, a user can determine if the oligonucleotide barcode is associated with a first solid support, a second solid support, a third solid support, a fourth solid support, a fifth solid support, a sixth solid support, a seventh solid support, or an eighth solid support.

In some embodiments, the cell label comprises a plurality of cell label portions and one or more linkers. In some embodiments, the cell label comprises a first cell label portion, a first linker, and a second cell label portion, optionally the cell label comprises a second linker and a third cell label portion, further optionally the cell label comprises a third linker and a fourth cell label portion. In some embodiments, the predetermined chamber indexing subsequence(s) comprise the first cell label portion, the second cell label portion, the third cell label portion, the fourth cell label portion, or any combination thereof. In some embodiments, the first, second, third, fourth, fifth, sixth, seventh, and/or eighth set of chamber indexing subsequence(s) comprises a set of less than about 960, about 864, about 768, about 672, about 576, about 480, about 384, about 288, about 192, about 96, or about 48, unique sequences distinct from the chamber indexing subsequence(s) of other sets of chamber indexing subsequence(s). In some embodiments, the first cell label portion, the second cell label portion, the third cell label portion, the fourth cell label portion, or any combination thereof, are selected from a set of less than about 480, about 384, about 288, about 192, about 96, or about 48, unique sequences. In some embodiments, based on the sequence(s) of the first cell label portion, the second cell label portion, the third cell label portion, the fourth cell label portion, or any combination thereof, of a sequencing read derived from an oligonucleotide barcode, or a product thereof, a user can determine if the oligonucleotide barcode is associated with a first solid support, a second solid support, a third solid support, a fourth solid support, a fifth solid support, a sixth solid support, a seventh solid support, or an eighth solid support.

Disclosed herein include methods. In some embodiments, the method comprises: distributing a plurality of precursor first solid supports and a first population of first oligonucleotides to a first plurality of first partitions, wherein colocalized solid supports and first oligonucleotides become associated; distributing a plurality of precursor second solid supports and a second population of first oligonucleotides to a first plurality of second partitions, wherein colocalized solid supports and first oligonucleotides become associated; pooling the plurality of precursor first solid supports associated with first oligonucleotides; pooling the plurality of precursor second solid supports associated with first oligonucleotides; distributing the plurality of precursor first solid supports associated with first oligonucleotides and a first population of second oligonucleotides to a second plurality of first partitions, wherein colocalized solid supports and second oligonucleotides become associated; distributing the plurality of precursor second solid supports associated with first oligonucleotides and a second population of second oligonucleotides to a second plurality of second partitions, wherein colocalized solid supports and second oligonucleotides become associated; pooling the plurality of precursor first solid supports associated with first and second oligonucleotides; pooling the plurality of precursor second solid supports associated with first and second oligonucleotides; distributing the plurality of precursor first solid supports associated with first and second oligonucleotides and a first population of third oligonucleotides to a third plurality of first partitions, wherein colocalized solid supports and third oligonucleotides become associated; and distributing the plurality of precursor second solid supports associated with first and second oligonucleotides and a second population of third oligonucleotides to a third plurality of second partitions, wherein colocalized solid supports and third oligonucleotides become associated. In some embodiments, the method comprises: pooling the plurality of precursor first solid supports associated with first, second, and third oligonucleotides to generate a plurality of first solid supports; and pooling the plurality of precursor second solid supports associated with first, second, and third oligonucleotides to generate a plurality of second solid supports.

In some embodiments, (i) the first and second populations of first oligonucleotides are the same, the first and second populations of second oligonucleotides are the same, and the first and second populations of third oligonucleotides are different; (ii) the first and second populations of first oligonucleotides are the same, the first and second populations of second oligonucleotides are different, and the first and second populations of third oligonucleotides are the same; (iii) the first and second populations of first oligonucleotides are the same, the first and second populations of second oligonucleotides are different, and the first and second populations of third oligonucleotides are different; (iv) the first and second populations of first oligonucleotides are different, the first and second populations of second oligonucleotides are different, and the first and second populations of third oligonucleotides are different; (v) the first and second populations of first oligonucleotides are different, the first and second populations of second oligonucleotides are different, and the first and second populations of third oligonucleotides are the same; (vi) the first and second populations of first oligonucleotides are different, the first and second populations of second oligonucleotides are the same, and the first and second populations of third oligonucleotides are different; or (vii) the first and second populations of first oligonucleotides are different, the first and second populations of second oligonucleotides are the same, and the first and second populations of third oligonucleotides are the same.

In some embodiments, the first and second populations of first oligonucleotides each comprise about 192 first cell label portions with distinct sequences. In some embodiments, the first and second populations of second oligonucleotides each comprise about 192 second cell label portions with distinct sequences. In some embodiments, the first and second populations of third oligonucleotides each comprise about 192 third cell label portions with distinct sequences.

n some embodiments, the first and second populations of first oligonucleotides are the same; the first and second populations of second oligonucleotides are the same; and the first and second populations of third oligonucleotides are different. In some embodiments, the first and second populations of third oligonucleotides comprise non-overlapping subsets of the cell label portion sequences of the first and second populations of first oligonucleotides and/or the first and second populations of second oligonucleotides.

In some embodiments, the first and second populations of first oligonucleotides each comprise about 384 first cell label portions with distinct sequences. In some embodiments, the first and second populations of second oligonucleotides each comprise about 384 second cell label portions with distinct sequences. In some embodiments, the first and second populations of third oligonucleotides each comprise about 48 third cell label portions with distinct sequences.

In some embodiments, the method comprises: distributing the plurality of precursor first solid supports associated with first, second, and third oligonucleotides and fourth oligonucleotides to a fourth plurality of first partitions, wherein colocalized solid supports and fourth oligonucleotides become associated; distributing the plurality of precursor second solid supports associated with first, second, and third oligonucleotides and a fifth oligonucleotides to a fourth plurality of second partitions, wherein colocalized solid supports and fifth oligonucleotides become associated; pooling the plurality of precursor first solid supports associated with first, second, third, and fourth oligonucleotides to generate a plurality of first solid supports; and pooling the plurality of precursor second solid supports associated with first, second, third, and fifth oligonucleotides to generate a plurality of second solid supports.

In some embodiments, the first and second populations of first oligonucleotides are the same; the first and second populations of second oligonucleotides are the same; the first and second populations of third oligonucleotides are the same; and the fifth oligonucleotides and sixth oligonucleotides are different.

In some embodiments, the first oligonucleotide comprises a first cell label portion and a first linker, or complements thereof. In some embodiments, the second oligonucleotide comprises a first linker, a second cell label portion, and a second linker, or complements thereof. In some embodiments, the third oligonucleotide comprises a second linker and a third cell label portion, or complements thereof, optionally the second oligonucleotide further comprises a third linker or complement thereof. In some embodiments, the fourth oligonucleotide comprises a third linker and a fourth cell label portion, or complements thereof.

In some embodiments, the first oligonucleotide and the second oligonucleotide are configured to connect via the first linker. In some embodiments, the second oligonucleotide and the third oligonucleotide are configured to connect via the second linker. In some embodiments, the third oligonucleotide and the fourth oligonucleotide are configured to connect via the third linker. In some embodiments, the plurality of first solid supports and/or the plurality of second solid supports comprise at least about 1000, about 10000, about 100000, about 1000000, about 7000000, about 10000000, about 56000000, unique cell label sequences. In some embodiments, the sequence of the first second cell label portion, the second cell label portion, and/or the third cell label portion is the same. In some embodiments, the sequence of the first second cell label portion, the second cell label portion, and/or the third cell label portion is different. In some embodiments, the first oligonucleotide, the second oligonucleotide, the third oligonucleotide, and/or the fourth oligonucleotide are single-stranded, double-stranded, and/or comprise one or two single-stranded overhangs. In some embodiments, the first solid supports and second solid supports each comprise a plurality of oligonucleotide barcodes each comprising a cell label sequence, wherein each cell label sequence comprises predetermined chamber indexing subsequence(s).

In some embodiments, oligonucleotide barcodes associated with plurality of first solid supports have a first predetermined chamber indexing subsequence(s), oligonucleotide barcodes associated with plurality of second solid supports have a second predetermined chamber indexing subsequence(s), and the first predetermined chamber indexing subsequence(s) and the second predetermined chamber indexing subsequence(s) are different.

In some embodiments, the first predetermined chamber indexing subsequence(s) of the plurality of first solid supports are selected from a first set of chamber indexing subsequence(s), the second predetermined chamber indexing subsequence(s) of the plurality of second solid supports are selected from a second set of chamber indexing subsequence(s), and each of the chamber indexing subsequence(s) of first set of chamber indexing subsequence(s) differ from the chamber indexing subsequence(s) of the second set of chamber indexing subsequence(s). The methods provided herein can generate the compositions provided herein.

In some embodiments, the method comprises: providing a microwell array comprising a plurality of chambers, wherein each chamber comprises a plurality of partitions; partitioning the plurality of first solid supports and the plurality of second solid supports to the partitions of an identified first chamber and identified second chamber of the plurality of chambers, respectively. In some embodiments, distributing oligonucleotides to a plurality of partitions comprises providing a plurality of partitions comprising said oligonucleotides. In some embodiments, the plurality of partitions comprise a 384-well plate, a 288-well plate, a 192-well plate, a 96-well plate or a 48-well plate.

In some embodiments, the solid support comprises a synthetic particle. In some embodiments, at least one oligonucleotide barcode of the plurality of oligonucleotide barcodes is immobilized on the synthetic particle, partially immobilized on the synthetic particle, enclosed in the synthetic particle, partially enclosed in the synthetic particle, or a combination thereof. In some embodiments, the synthetic particle is disruptable. In some embodiments, the synthetic particle comprises a bead. In some embodiments, the bead comprises a Sepharose bead, a streptavidin bead, an agarose bead, a magnetic bead, a conjugated bead, a protein A conjugated bead, a protein G conjugated bead, a protein A/G conjugated bead, a protein L conjugated bead, an oligo(dT) conjugated bead, a silica bead, a silica-like bead, an anti-biotin microbead, an anti-fluorochrome microbead, or any combination thereof. In some embodiments, the synthetic particle comprises a material selected from the group consisting of polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, and any combination thereof. In some embodiments, the synthetic particle comprises a disruptable hydrogel particle.

There are provided, in some embodiments, methods of assigning sequencing data to chambers. In some embodiments, the method comprises: providing a microwell array comprising a plurality of chambers, wherein each chamber comprises a plurality of partitions; partitioning each of two or more pluralities of solid supports to the partitions of an identified chamber of the plurality of chambers, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s); partitioning each of two or more populations of single cells to the partitions of an identified chamber of the plurality of chambers, wherein each of the two or more populations of single cells comprises a plurality of single cells, wherein the single cells comprise copies of a nucleic acid target, wherein single cells of the same population are situated within the same chamber, and wherein single cells of different populations are situated within different chambers; barcoding the copies of a nucleic acid target from at least one of the plurality of single cells of at least one population of single cells using the plurality of oligonucleotide barcodes to generate a plurality of barcoded nucleic acid targets; obtaining sequencing data comprising a plurality of sequencing reads of the plurality of barcoded nucleic acid targets, or products thereof; identifying the chamber indexing subsequence(s) of each cell label sequence in the sequencing data; and assigning each of the plurality of sequencing reads to a chamber of the plurality of chambers based on the chamber indexing subsequence(s) of each cell label sequence in the sequencing data.

There are provided, in some embodiments, methods of assigning sequencing data to chambers. In some embodiments, the method comprises: providing a microwell array comprising a plurality of chambers, wherein each chamber comprises a plurality of partitions, wherein each chamber comprises a predetermined plurality of solid supports selected from two or more pluralities of solid supports, wherein the solid supports are situated within the partitions of said chambers, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s); partitioning each of two or more populations of single cells to the partitions of an identified chamber of the plurality of chambers, wherein each of the two or more populations of single cells comprises a plurality of single cells, wherein the single cells comprise copies of a nucleic acid target, wherein single cells of the same population are situated within the same chamber, and wherein single cells of different populations are situated within different chambers; barcoding the copies of a nucleic acid target from at least one of the plurality of single cells of at least one population of single cells using the plurality of oligonucleotide barcodes to generate a plurality of barcoded nucleic acid targets; obtaining sequencing data comprising a plurality of sequencing reads of the plurality of barcoded nucleic acid targets, or products thereof; identifying the chamber indexing subsequence(s) of each cell label sequence in the sequencing data; and assigning each of the plurality of sequencing reads to a chamber of the plurality of chambers based on the chamber indexing subsequence(s) of each cell label sequence in the sequencing data.

Provided herein include methods of assigning sequencing data to a population of single cells. In some embodiments, the method comprises: providing a microwell array comprising a plurality of chambers, wherein each chamber comprises a plurality of partitions; partitioning each of two or more pluralities of solid supports to the partitions of an identified chamber of the plurality of chambers, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s); partitioning each of two or more populations of single cells to the partitions of an identified chamber of the plurality of chambers, wherein each of the two or more populations of single cells comprises a plurality of single cells, wherein the single cells comprise copies of a nucleic acid target, wherein single cells of the same population are situated within the same chamber, and wherein single cells of different populations are situated within different chambers; barcoding the copies of a nucleic acid target from at least one of the plurality of single cells of at least one population of single cells using the plurality of oligonucleotide barcodes to generate a plurality of barcoded nucleic acid targets; obtaining sequencing data comprising a plurality of sequencing reads of the plurality of barcoded nucleic acid targets, or products thereof; identifying the chamber indexing subsequence(s) of each cell label sequence in the sequencing data; assigning each of the plurality of sequencing reads to a chamber of the plurality of chambers based on the chamber indexing subsequence(s) of each cell label sequence in the sequencing data; and assigning each of the plurality of sequencing reads to a population of the two or more populations of single cells based on the chamber assigned to the sequencing read.

There are provided, in some embodiments, methods of assigning sequencing data to a population of single cells. In some embodiments, the method comprises: providing a microwell array comprising a plurality of chambers, wherein each chamber comprises a plurality of partitions, wherein each chamber comprises a predetermined plurality of solid supports selected from two or more pluralities of solid supports, wherein the solid supports are situated within the partitions of said chambers, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s); partitioning each of two or more populations of single cells to the partitions of an identified chamber of the plurality of chambers, wherein each of the two or more populations of single cells comprises a plurality of single cells, wherein the single cells comprise copies of a nucleic acid target, wherein single cells of the same population are situated within the same chamber, and wherein single cells of different populations are situated within different chambers; barcoding the copies of a nucleic acid target from at least one of the plurality of single cells of at least one population of single cells using the plurality of oligonucleotide barcodes to generate a plurality of barcoded nucleic acid targets; obtaining sequencing data comprising a plurality of sequencing reads of the plurality of barcoded nucleic acid targets, or products thereof; identifying the chamber indexing subsequence(s) of each cell label sequence in the sequencing data; and assigning each of the plurality of sequencing reads to a chamber of the plurality of chambers based on the chamber indexing subsequence(s) of each cell label sequence in the sequencing data; and assigning each of the plurality of sequencing reads to a population of the two or more populations of single cells based on the chamber assigned to the sequencing read.

Some embodiments provide methods of associating sequencing data and phenotypic data of a population of single cells. In some embodiments, the method comprises: obtaining phenotypic data of each of two or more populations of single cells; providing a microwell array comprising a plurality of chambers, wherein each chamber comprises a plurality of partitions; partitioning each of two or more pluralities of solid supports to the partitions of an identified chamber of the plurality of chambers, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s); partitioning each of two or more populations of single cells to the partitions of an identified chamber of the plurality of chambers, wherein each of the two or more populations of single cells comprises a plurality of single cells, wherein the single cells comprise copies of a nucleic acid target, wherein single cells of the same population are situated within the same chamber, and wherein single cells of different populations are situated within different chambers; barcoding the copies of a nucleic acid target from at least one of the plurality of single cells of at least one population of single cells using the plurality of oligonucleotide barcodes to generate a plurality of barcoded nucleic acid targets; obtaining sequencing data comprising a plurality of sequencing reads of the plurality of barcoded nucleic acid targets, or products thereof; identifying the chamber indexing subsequence(s) of each cell label sequence in the sequencing data; and associating the sequencing data and the phenotypic data of at least one population of single cells based on the chamber indexing subsequence(s) of each cell label sequence in the sequencing data.

There are provided, in some embodiments, methods of associating sequencing data and phenotypic data of a population of single cells. In some embodiments, the method comprises: obtaining phenotypic data of each of two or more populations of single cells; providing a microwell array comprising a plurality of chambers, wherein each chamber comprises a plurality of partitions, wherein each chamber comprises a predetermined plurality of solid supports selected from two or more pluralities of solid supports, wherein the solid supports are situated within the partitions of said chambers, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s); partitioning each of two or more populations of single cells to the partitions of an identified chamber of the plurality of chambers, wherein each of the two or more populations of single cells comprises a plurality of single cells, wherein the single cells comprise copies of a nucleic acid target, wherein single cells of the same population are situated within the same chamber, and wherein single cells of different populations are situated within different chambers; barcoding the copies of a nucleic acid target from at least one of the plurality of single cells of at least one population of single cells using the plurality of oligonucleotide barcodes to generate a plurality of barcoded nucleic acid targets; obtaining sequencing data comprising a plurality of sequencing reads of the plurality of barcoded nucleic acid targets, or products thereof; identifying the chamber indexing subsequence(s) of each cell label sequence in the sequencing data; and associating the sequencing data and the phenotypic data of at least one population of single cells based on the chamber indexing subsequence(s) of each cell label sequence in the sequencing data.

The method can comprise: assigning each of the plurality of sequencing reads to a population of the two or more populations of single cells based on the chamber assigned to the sequencing read. The method can comprise: obtaining phenotypic data of each of two or more populations of single cells; and associating the sequencing data and the phenotypic data of at least one population of single cells based on the chamber indexing subsequence(s) of each cell label sequence in the sequencing data.

In some embodiments, partitioning each of two or more pluralities of solid supports to the partitions of an identified chamber of the plurality of chambers can comprise partitioning a predetermined plurality of solid supports selected from two or more pluralities of solid supports to an identified chamber of the plurality of chambers. In some embodiments, barcoding the copies of a nucleic acid target comprises: contacting the plurality of oligonucleotide barcodes with the copies of a nucleic acid target for hybridization; and extending the plurality of oligonucleotide barcodes hybridized to the copies of a nucleic acid target to generate a plurality of barcoded nucleic acid targets.

A partition can be a microwell having a volume ranging from about 1,000 μm³ to about 786,000 μm³. The oligonucleotide barcodes each can comprise a molecular label sequence. In some embodiments, oligonucleotide barcodes of the same plurality of solid supports can comprise the same chamber indexing subsequence(s), and wherein oligonucleotide barcodes of different pluralities of solid supports can comprise different chamber indexing subsequence(s). Each cell label of the plurality of oligonucleotide barcodes can comprise at least 6 nucleotides. The cell label can comprise a plurality of portions and one or more linkers. The cell label can comprise a first cell label portion, a first linker, and a second cell label portion, optionally the cell label comprises a second linker and a third cell label portion, further optionally the cell label comprises a third linker and a fourth cell label portion. The first cell label portion can comprise the chamber indexing subsequence(s). In some embodiments, chamber indexing subsequence(s) can be 2-15 nucleotides in length. The two or more pluralities of solid supports can comprise about 2 to about 100 different pluralities of solid supports. Oligonucleotide barcodes of the same plurality of solid supports can comprise the same chamber indexing subsequence(s), and oligonucleotide barcodes of different pluralities of solid supports can comprise different chamber indexing subsequence(s).

The chamber indexing subsequence(s) can be selected from a diverse set of chamber indexing subsequence(s). The diverse set of chamber indexing subsequence(s) can comprise at least 20, at least 30, at least 40, at least 50, at least 60, at least 70, at least 80, at least 90, at least 100, at least 200, at least 300, at least 400, at least 500, at least 600, at least 700, at least 800, at least 900, at least 1,000, at least 2,000, at least 5,000, or more different chamber indexing subsequence(s). In some embodiments, the set of chamber indexing subsequence(s) is designed to have minimal sequence homology to the DNA or RNA sequences of the sample to be analyzed. In some embodiments, the sequences of the set of chamber indexing subsequence(s) are different from each other, or the complement thereof, by at least 1 nucleotide, at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, or more. In some embodiments, the sequences of the set of chamber indexing subsequence(s) are different from each other, or the complement thereof, by at least 3%, at least 5%, at least 8%, at least 10%, at least 15%, at least 20%, or more.

The populations of single cells can be distinct samples. Each population of the two or more populations of single cells can be a biological replicate sample, a technical replicate sample, a control sample, an experimental sample, or a combination thereof. The two or more populations of single cells can be derived from one or more samples separated on the basis of phenotypic data, optionally the plurality of single cells can comprise T cells, B cells, tumor cells, myeloid cells, blood cells, normal cells, fetal cells, maternal cells, or a mixture thereof. The plurality of chambers can be at least about 4 chambers, about 10 chambers, about 20 chambers, about 40 chambers, about 60 chambers, about 80 chambers, about 100 chambers, about 200 chambers, about 300 chambers, about 400 chambers, about 500 chambers, about 750 chambers, or about 1000 chambers. The plurality of partitions can be at least about 100 partitions, about 500 partitions, about 1000 partitions, about 5000 partitions, about 10000 partitions, about 25000 partitions, about 50000 partitions, about 75000 partitions, or about 100000 partitions.

In some embodiments, the sequencing of the cell label identifies the chamber of origin of each sequenced barcoded nucleic acid target, or products thereof, within the microwell array. A partition of the plurality of partitions can comprise a single cell of the plurality of single cells and a single solid support of the plurality of solid supports. The plurality of barcoded nucleic acid targets each can comprise a sequence complementary to at least a portion of the nucleic acid target and the molecular label. Each of the plurality of sequencing reads can comprise (1) a cell label sequence and (2) a molecular label sequence. Each oligonucleotide barcode can comprise a first universal sequence. The oligonucleotide barcode can comprise a target-binding region comprising a capture sequence. The target-binding region can comprise a gene-specific sequence, an oligo(dT) sequence, a random multimer, or any combination thereof. In some embodiments, partitioning a plurality of solid supports to the partitions of each of the plurality of chambers can comprise flow cytometrically depositing each plurality of solid supports into an identified chamber of the microwell array. In some embodiments, partitioning each of two or more populations of single cells to the partitions of an identified chamber of the plurality of chambers can comprise flow cytometrically depositing each population of single cells into an identified chamber of the microwell array. The method can comprise: aligning a sorting component of a flow cytometer with the microwell array.

The phenotypic data can comprise event data. The event data can comprise quantitative biological event data derived from a sorting device. The event data can comprise a side scatter signal, a forward scatter signal, one or more fluorescence signals, or any combination thereof. The method can comprise: correlation analysis of the phenotypic data and the sequencing data of the single cells. In some embodiments, the correlation analysis identifies one or more of the following: candidate biomarkers, candidate therapeutic agents, candidate doses of therapeutic agents, and/or cellular targets of candidate therapeutic agents. The method can comprise: lysing the one or more of the single cells. The viability of the single cells can be maintained for a period of time after partitioning and before lysis, optionally the period of time is at least about 10 min, 20 min, 30 min, 40 min, 50 min, 60 min, 100 min, 250 min, 500 min, 750 min, 1000 min, 2500 min, 5000 min, 7500 min, or 10000 min.

The solid support can comprise a synthetic particle. The solid support can comprise a planar surface. At least one of the plurality of oligonucleotide barcodes can be immobilized on, partially immobilized, enclosed in, or partially enclosed in the synthetic particle. The synthetic particle can be disruptable. The synthetic particle can comprise a bead. The bead can comprise a Sepharose bead, a streptavidin bead, an agarose bead, a magnetic bead, a conjugated bead, a protein A conjugated bead, a protein G conjugated bead, a protein A/G conjugated bead, a protein L conjugated bead, an oligo(dT) conjugated bead, a silica bead, a silica-like bead, an anti-biotin microbead, an anti-fluorochrome microbead, or any combination thereof. The synthetic particle can comprise a material selected from polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, and any combination thereof. The synthetic particle can comprise a disruptable hydrogel particle.

### Methods of Determining the Copy Number of a Nucleic Acid Target

The methods provided herein can comprise determining the copy number of the nucleic acid target in one or more of the plurality of single cells of at least one population of single cells. Determining the copy number of the nucleic acid target in one or more of the plurality of single cells can comprise determining the copy number of the nucleic acid target in the plurality of single cells based on the number of molecular labels with distinct sequences, complements thereof, or a combination thereof, associated with the plurality of barcoded nucleic acid targets, or products thereof. The method can comprise: contacting random primers with the plurality of barcoded nucleic acid targets, wherein each of the random primers comprises a second universal sequence, or a complement thereof; and extending the random primers hybridized to the plurality of barcoded nucleic acid targets to generate a plurality of extension products. The method can comprise: amplifying the plurality of extension products using primers capable of hybridizing to the first universal sequence or complements thereof, and primers capable of hybridizing the second universal sequence or complements thereof, thereby generating a first plurality of barcoded amplicons. Amplifying the plurality of extension products can comprise adding sequences of binding sites of sequencing primers and/or sequencing adaptors, complementary sequences thereof, and/or portions thereof, to the plurality of extension products. The method can comprise: determining the copy number of the nucleic acid target in one or more of the plurality of single cells based on the number of molecular labels with distinct sequences associated with the first plurality of barcoded amplicons, or products thereof. In some embodiments, determining the copy number of the nucleic acid target in one or more of the plurality of single cells can comprise determining the number of each of the plurality of nucleic acid targets in one or more of the plurality of single cells based on the number of the molecular labels with distinct sequences associated with barcoded amplicons of the first plurality of barcoded amplicons comprising a sequence of the each of the plurality of nucleic acid targets. The sequence of the each of the plurality of nucleic acid targets can comprise a subsequence of the each of the plurality of nucleic acid targets. The sequence of the nucleic acid target in the first plurality of barcoded amplicons can comprise a subsequence of the nucleic acid target. The method can comprise: amplifying the first plurality of barcoded amplicons using primers capable of hybridizing to the first universal sequence or complements thereof, and primers capable of hybridizing the second universal sequence or complements thereof, thereby generating a second plurality of barcoded amplicons. Amplifying the first plurality of barcoded amplicons can comprise adding sequences of binding sites of sequencing primers and/or sequencing adaptors, complementary sequences thereof, and/or portions thereof, to the first plurality of barcoded amplicons. The method can comprise: determining the copy number of the nucleic acid target in one or more of the plurality of single cells based on the number of molecular labels with distinct sequences associated with the second plurality of barcoded amplicons, or products thereof. The first plurality of barcoded amplicons and/or the second plurality of barcoded amplicons can comprise whole transcriptome amplification (WTA) products.

The method can comprise: synthesizing a third plurality of barcoded amplicons using the plurality of barcoded nucleic acid targets as templates to generate a third plurality of barcoded amplicons. Synthesizing a third plurality of barcoded amplicons can comprise performing polymerase chain reaction (PCR) amplification of the plurality of the barcoded nucleic acid targets. Synthesizing a third plurality of barcoded amplicons can comprise PCR amplification using primers capable of hybridizing to the first universal sequence, or a complement thereof, and a target-specific primer. The method can comprise: obtaining sequence data of the third plurality of barcoded amplicons, or products thereof, and optionally obtaining the sequence information comprises attaching sequencing adaptors to the third plurality of barcoded amplicons, or products thereof. The method can comprise: determining the copy number of the nucleic acid target in one or more of the plurality of single cells based on the number of molecular labels with distinct sequences associated with the third plurality of barcoded amplicons, or products thereof.

The nucleic acid target can comprise a nucleic acid molecule. The nucleic acid molecule can comprise ribonucleic acid (RNA), messenger RNA (mRNA), microRNA, small interfering RNA (siRNA), RNA degradation product, RNA comprising a poly(A) tail, or any combination thereof. The nucleic acid target can comprise a sample indexing oligonucleotide, and optionally the sample indexing oligonucleotide comprises a sample indexing sequence, and sample indexing sequences of at least two sample indexing compositions of a plurality of sample indexing compositions comprise different sequences. The nucleic acid target can comprise a cellular component-binding reagent specific oligonucleotide. A cellular component-binding reagent specific oligonucleotide can comprise a unique identifier sequence for a cellular component-binding reagent. In some embodiments of the methods and compositions provided herein the nucleic acid target is a binding reagent oligonucleotide (e.g., antibody oligonucleotide (“AbOligo” or “AbO”), binding reagent oligonucleotide, cellular component-binding reagent specific oligonucleotide, sample indexing oligonucleotide). Some embodiments disclosed herein provide a plurality of compositions each comprising a cellular component binding reagent (such as a protein binding reagent) that is conjugated with an oligonucleotide (e.g., a binding reagent oligonucleotide), wherein the oligonucleotide comprises a unique identifier for the cellular component binding reagent that it is conjugated with. Cellular component binding reagents (such as barcoded antibodies) and their uses (such as sample indexing of cells) have been described in US2018/0088112 and US2018/0346970; the content of each of these is incorporated herein by reference in its entirety.

Extending the plurality of oligonucleotide barcodes can comprise extending the plurality of oligonucleotide barcodes using a reverse transcriptase and/or a DNA polymerase lacking at least one of 5′ to 3′ exonuclease activity and 3′ to 5′ exonuclease activity. The DNA polymerase can comprise a Klenow Fragment. The reverse transcriptase can comprise a viral reverse transcriptase, optionally wherein the viral reverse transcriptase is a murine leukemia virus (MLV) reverse transcriptase or a Moloney murine leukemia virus (MMLV) reverse transcriptase. The first universal sequence and the second universal sequence can be the same. The first universal sequence and the second universal sequence can be different. The first universal sequence, and/or the second universal sequence can comprise the binding sites of sequencing primers and/or sequencing adaptors, complementary sequences thereof, and/or portions thereof. The sequencing adaptors can comprise a P5 sequence, a P7 sequence, complementary sequences thereof, and/or portions thereof. The sequencing primers can comprise a Read 1 sequencing primer, a Read 2 sequencing primer, complementary sequences thereof, and/or portions thereof. At least 10 of the plurality of oligonucleotide barcodes can comprise different molecular label sequences. Each molecular label of the plurality of oligonucleotide barcodes can comprise at least 6 nucleotides.

### Particle Analyzers

The phenotypic data can comprise data derived from a sorting device (e.g., a flow cytometer). The phenotypic data can comprise event data. The event data can comprise quantitative biological event data derived from a sorting device. The event data can comprise a side scatter signal, a forward scatter signal, one or more fluorescence signals, or any combination thereof. As used herein, the terms “event” or “event data” shall be given their ordinary meanings and shall also be interchangeable with one another, and shall also refer to the data measured from a single particle, such as cells or synthetic particles. Typically, the data measured from a single particle include a number of parameters, including one or more light scattering parameters, and at least one fluorescence intensity parameters. Thus, each event is represented as a vector of parameter measurements, wherein each measured parameter corresponds to one dimension of the data space. In some biological applications, event data may correspond to quantitative biological data indicating expression of a particular protein or gene. The method can comprise: correlation analysis of the phenotypic data and the sequencing data of the single cells. The correlation analysis can identify one or more of the following: candidate biomarkers, candidate therapeutic agents, candidate doses of therapeutic agents, and/or cellular targets of candidate therapeutic agents.

Particle analyzers, such as flow and scanning cytometers, are analytical tools that enable the characterization of particles (e.g., single cells) on the basis of optical parameters such as light scatter and fluorescence. In a flow cytometer, for example, particles, such as molecules, analyte-bound beads, or individual cells, in a fluid suspension are passed by a detection region in which the particles are exposed to an excitation light, typically from one or more lasers, and the light scattering and fluorescence properties of the particles are measured. Particles or components thereof typically are labeled with fluorescent dyes to facilitate detection. A multiplicity of different particles or components may be simultaneously detected by using spectrally distinct fluorescent dyes to label the different particles or components. In some implementations, a multiplicity of photodetectors, one for each of the scatter parameters to be measured, and one for each of the distinct dyes to be detected are included in the analyzer. The data obtained comprise the signals measured for each of the light scatter parameters and the fluorescence emissions.

The parameters measured using a flow cytometer typically include the excitation light that is scattered by the particle along a mostly forward direction, referred to as forward scatter (FSC), the excitation light that is scattered by the particle in a mostly sideways direction, referred to as side scatter (SSC), and the light emitted from fluorescent molecules in one or more channels (range of frequencies) of the spectrum, referred to as FL1, FL2, etc., or by the fluorescent dye that is primarily detected in that channel. Different cell types can be identified by the scatter parameters and the fluorescence emissions resulting from labeling various cell proteins with dye-labeled antibodies.

Both flow and scanning cytometers are commercially available from, for example, BD Biosciences (San Jose, Calif.). Flow cytometry is described in, for example, Landy et al. (eds.), Clinical Flow Cytometry, Annals of the New York Academy of Sciences Volume 677 (1993); Bauer et al. (eds.), Clinical Flow Cytometry: Principles and Applications, Williams & Wilkins (1993); Ormerod (ed.), Flow Cytometry: A Practical Approach, Oxford Univ. Press (1994); Jaroszeski et al. (eds.), Flow Cytometry Protocols, Methods in Molecular Biology No. 91, Humana Press (1997); and Practical Shapiro, Flow Cytometry, 4th ed., Wiley-Liss (2003); all incorporated herein by reference. Fluorescence imaging microscopy is described in, for example, Pawley (ed.), Handbook of Biological Confocal Microscopy, 2nd Edition, Plenum Press (1989), incorporated herein by reference.

The data obtained from an analysis of cells (or other particles) by multi-color flow cytometry are multidimensional, wherein each cell corresponds to a point in a multidimensional space defined by the parameters measured. Populations of cells or particles are identified as clusters of points in the data space. The identification of clusters and, thereby, populations can be carried out manually by drawing a gate around a population displayed in one or more 2-dimensional plots, referred to as “scatter plots” or “dot plots,” of the data. Alternatively, clusters can be identified, and gates that define the limits of the populations, can be determined automatically. Examples of methods for automated gating have been described in, for example, U.S. Pat. Nos. 4,845,653; 5,627,040; 5,739,000; 5,795,727; 5,962,238; 6,014,904; and 6,944,338; and U.S. Pat. Pub. No. 2012/0245889, each incorporated herein by reference.

Flow cytometry is a valuable method for the analysis and isolation of biological particles such as cells and constituent molecules. As such it has a wide range of diagnostic and therapeutic applications. The method utilizes a fluid stream to linearly segregate particles such that they can pass, single file, through a detection apparatus. Individual cells can be distinguished according to their location in the fluid stream and the presence of detectable markers. Thus, a flow cytometer can be used to produce a diagnostic profile of a population of biological particles.

Isolation of biological particles has been achieved by adding a sorting or collection capability to flow cytometers. Particles in a segregated stream, detected as having one or more desired characteristics, are individually isolated from the sample stream by mechanical or electrical removal. This method of flow sorting has been used to sort cells of different types, to separate sperm bearing X and Y chromosomes for animal breeding, to sort chromosomes for genetic analysis, and to isolate particular organisms from complex biological population.

In flow cytometry sorting, the use of index-sorting implies additional information is available that links the individual cell events to their destination locations in a plate or slide holder. This information may be used post-acquisition to do additional analysis of where cells are physically located on a plate device. It also allows users to see where those cells are located on bi-variate plots.

Index sorting is cell sorting where the sorting device may record the sort decision for each event (typically a cell or other particle suspended in a flow stream) and the data is available for post sort analysis. Typically index sorting is performed by detecting a property of a particle (such as color) and directing the particle into a collection plate. The plate may include several plate destinations (e.g., well locations). The soring may include directing the particle to a particular plate location (e.g., well) within the plate. The sorting device may record, in association with an identifier for the event, the destination plate and/or well location. Each sorted event thus has all the measurements from the detectors (PMTs, photodiodes) along with the well location and sort destination. A user can examine a sorted cell's data and correlate it with subsequent operations on the plate (e.g., gene expression derived from sequencing the sorted cells).

A sort mode may be specified for a given sample. The sort mode includes the parameters to control which events are sorted. For example, upon receiving a sample at the sorting device, the sorting device may receive a sort mode to control what properties to use for sorting and where detected values for those properties should be sorted. Sort modes may include a purity mode which may configure the cell sorter to ensure that the desired cell type and only the desired cell type are in the gate. Sort modes may include single cell mode which may configure the cell sorter to ensure that only a single cell is within the droplet. Since there can be uncertainty around the drop boundary of which droplet a cell may be in, often following drops are not sorted even if they could be. For single cell sorting, this may be desirable for certain experiments such as genomic based assays where users may want to correlate gene expression with flow cytometry derived measurements. In such instances, if there were multiple cells within the well, there would be ambiguity as to which cell the gene sequences came from. The sort mode may configure the cell sorter by setting a mask that examines where events fall within the droplet, and surrounding droplets. Some single cell sort mode configurations may include a state machine or examination of a queue of events that fall within a droplet.

In some implementations, a target gate may be used to identify events of interest. A target gate may be provided by selecting an area on a two dimensional plot. Events that are detected with property values within the selected area for the two dimensions are considered within the target gate and may be sorted to a particular location. An event may be within the target gate but under certain sorting modes (e.g., a purity or single cell mode), the event may not be sorted correctly such as if another event is within the same drop in the fluidic stream as the event. In some implementations, this may be referred to as entrainment or cohesion.

As part of recording the sort decisions, the sort electronics of the sorting device may transmit the sort destination along with the event raw data. Event raw data may include a detected property for the event (e.g., reflected light values, fluorescence information, light scatter information, time of the event, a sequence number for the event, sorting device operational characteristics at the time the event was analyzed (e.g., temperature, flow rate, sort mode, etc.), or the like). For index sorting, the current tray, plate, microscope slide, or other physical medium with spatially separated pools where drops including cells may be deposited, coordinates of the location where a cell for a particular event was deposited may also be transmitted.

Methods disclosed herein can, in some embodiments, include enriching a sample comprising a plurality of cells for cells of interest to produce an enriched cell sample comprising a plurality of single cells for analysis as provided herein. Enriching the sample can comprise focusing cells of interest in the sample; isolating one or more cells of interest in the enriched cell sample with a flow cytometer; and obtaining sequence information of one or more polynucleotides from each of the one or more isolated cells as described herein. Various focusing methods and techniques can be used, for example, hydrodynamic focusing, magnetic field focusing, electric field focusing, gravitational field focusing, optical field focusing, and any combination thereof. In some embodiments, enriching the sample includes depleting cells not of interest in the sample. In some embodiments, enriching the sample includes both acoustic focusing and depleting cells not of interest in the sample. In some embodiments, one or more of cells not of interest, interfering cells and debris in the sample can be depleted, for example using magnetic depletion.

### Compositions and Kits

Disclosed herein include compositions. In some embodiments, the composition comprises: a microwell array, wherein the microwell array comprises a plurality of chambers, wherein each chamber comprises a plurality of partitions, wherein each partition is a microwell having a volume ranging from about 1,000 μm³ to about 786,000 μm³. The composition can comprise: a cartridge, wherein the cartridge comprises at least one of: an inlet port, an outlet port, a pump, a valve, a vent, a reservoir, a sample collection chamber, a temperature control apparatus, or any combination thereof.

Disclosed herein include compositions. In some embodiments, the composition comprises: a cartridge, wherein the cartridge comprises at least one of: an inlet port, an outlet port, a pump, a valve, a vent, a reservoir, a sample collection chamber, a temperature control apparatus, or any combination thereof, wherein the cartridge comprises a microwell array, wherein the microwell array comprises a plurality of chambers, wherein each chamber comprises a plurality of partitions, wherein each partition is a microwell having a volume ranging from about 1,000 μm³ to about 786,000 μm³.

Disclosed herein include compositions. In some embodiments, the composition comprises: two or more pluralities of solid supports, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes of the same plurality of solid supports comprise the same chamber indexing subsequence(s), and wherein oligonucleotide barcodes of different pluralities of solid supports comprise different chamber indexing subsequence(s).

A partition of the plurality of partitions comprises a single solid support of a plurality of solid supports, wherein the solid supports each comprise a plurality of oligonucleotide barcodes each comprising a cell label sequence, wherein each cell label sequence comprises predetermined chamber indexing subsequence(s), wherein oligonucleotide barcodes associated with the same solid support comprise the same cell label sequence, wherein oligonucleotide barcodes associated with different solid supports comprise different cell label sequences, wherein oligonucleotide barcodes situated within the same chamber comprise the same chamber indexing subsequence(s), and wherein oligonucleotide barcodes situated within different chambers comprise different chamber indexing subsequence(s).

The composition can comprise: a chamber indexing subsequence(s) lookup table. In some embodiments, the chamber indexing subsequence(s) lookup table identifies the chamber indexing subsequence(s) associated with each solid support distributed in each microwell of the array.

The cartridge can be configured to maintain the viability of single cells partitioned within said microwells, optionally the period of time is at least about 10 min, 20 min, 30 min, 40 min, 50 min, 60 min, 100 min, 250 min, 500 min, 750 min, 1000 min, 2500 min, 5000 min, 7500 min, or 10000 min. The cartridge can comprise a transparent window for optical imaging of the microwells. The composition can comprise: an imaging system configured to capture and process images of all or a portion of the microwells, wherein the imaging system further comprises an illumination subsystem, an imaging subsystem, and a processor. The imaging system can be configured to perform bright-field, dark-field, fluorescence, or quantitative phase imaging.

The composition can comprise: a buffer. The composition can comprise: one or more reagents for a reverse transcription reaction, one or more reagents for an amplification reaction, or both. The oligonucleotide barcodes each can comprise a molecular label sequence. Each cell label of the plurality of oligonucleotide barcodes can comprise at least 6 nucleotides. The cell label can comprise a plurality of portions and one or more linkers. The cell label can comprise a first cell label portion, a first linker, and a second cell label portion, optionally the cell label comprises a second linker and a third cell label portion, further optionally the cell label comprises a third linker and a fourth cell label portion. The first cell label portion can comprise the chamber indexing subsequence(s). The chamber indexing subsequence(s) can be 2-15 nucleotides in length. The plurality of chambers can be at least about 4 chambers, about 10 chambers, about 20 chambers, about 40 chambers, about 60 chambers, about 80 chambers, about 100 chambers, about 200 chambers, about 300 chambers, about 400 chambers, about 500 chambers, about 750 chambers, or about 1000 chambers. The plurality of partitions can be at least about 100 partitions, about 500 partitions, about 1000 partitions, about 5000 partitions, about 10000 partitions, about 25000 partitions, about 50000 partitions, about 75000 partitions, or about 100000 partitions.

The composition can comprise: a plurality of solid supports each comprising a plurality of oligonucleotide barcodes. The oligonucleotide barcodes each can comprise a molecular label and a cell label. In some embodiments, oligonucleotide barcodes associated with the same solid support can comprise the same cell label sequence, and wherein oligonucleotide barcodes associated with different solid supports can comprise different cell label sequences. Each oligonucleotide barcode can comprise a first universal sequence. The oligonucleotide barcode can comprise a target-binding region comprising a capture sequence. The target-binding region can comprise a gene-specific sequence, an oligo(dT) sequence, a random multimer, or any combination thereof. Each molecular label of the plurality of oligonucleotide barcodes can comprise at least 6 nucleotides. The solid support can comprise a planar surface. The solid support can comprise a synthetic particle. At least one oligonucleotide barcode of the plurality of oligonucleotide barcodes can be immobilized on the synthetic particle, partially immobilized on the synthetic particle, enclosed in the synthetic particle, partially enclosed in the synthetic particle, or a combination thereof. The synthetic particle can be disruptable. The synthetic particle can comprise a bead. The bead can comprise a Sepharose bead, a streptavidin bead, an agarose bead, a magnetic bead, a conjugated bead, a protein A conjugated bead, a protein G conjugated bead, a protein A/G conjugated bead, a protein L conjugated bead, an oligo(dT) conjugated bead, a silica bead, a silica-like bead, an anti-biotin microbead, an anti-fluorochrome microbead, or any combination thereof. The synthetic particle can comprise a material selected from polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, and a combination thereof. The synthetic particle can comprise a disruptable hydrogel particle. In some embodiments, the composition further comprises instructions for use.

### Terminology

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

One skilled in the art will appreciate that, for this and other processes and methods disclosed herein, the functions performed in the processes and methods can be implemented in differing order. Furthermore, the outlined steps and operations are only provided as examples, and some of the steps and operations can be optional, combined into fewer steps and operations, or expanded into additional steps and operations without detracting from the essence of the disclosed embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

What is claimed is:

1. A method of assigning sequencing data to chambers, comprising:
providing a microwell array comprising a plurality of chambers, wherein each chamber comprises a plurality of partitions;
partitioning each of two or more pluralities of solid supports to the partitions of an identified chamber of the plurality of chambers, wherein the solid supports each comprise a plurality of oligonucleotide barcodes each comprising a cell label sequence, wherein each cell label sequence comprises predetermined chamber indexing subsequence(s),
wherein oligonucleotide barcodes associated with the same solid support comprise the same cell label sequence, wherein oligonucleotide barcodes associated with different solid supports comprise different cell label sequences,
wherein oligonucleotide barcodes situated within the same chamber comprise the same chamber indexing subsequence(s), and wherein oligonucleotide barcodes situated within different chambers comprise different chamber indexing subsequence(s);
partitioning each of two or more populations of single cells to the partitions of an identified chamber of the plurality of chambers,
wherein each of the two or more populations of single cells comprises a plurality of single cells, wherein the single cells comprise copies of a nucleic acid target,
wherein single cells of the same population are situated within the same chamber, and wherein single cells of different populations are situated within different chambers;
barcoding the copies of a nucleic acid target from at least one of the plurality of single cells of at least one population of single cells using the plurality of oligonucleotide barcodes to generate a plurality of barcoded nucleic acid targets;
obtaining sequencing data comprising a plurality of sequencing reads of the plurality of barcoded nucleic acid targets, or products thereof;
identifying the chamber indexing subsequence(s) of each cell label sequence in the sequencing data; and
assigning each of the plurality of sequencing reads to a chamber of the plurality of chambers based on the chamber indexing subsequence(s) of each cell label sequence in the sequencing data.

2. A method of assigning sequencing data to chambers, comprising:
providing a microwell array comprising a plurality of chambers, wherein each chamber comprises a plurality of partitions, wherein each chamber comprises a predetermined plurality of solid supports selected from two or more pluralities of solid supports, wherein the solid supports are situated within the partitions of said chambers, wherein the solid supports each comprise a plurality of oligonucleotide barcodes each comprising a cell label sequence, wherein each cell label sequence comprises predetermined chamber indexing subsequence(s),
wherein oligonucleotide barcodes associated with the same solid support comprise the same cell label sequence, wherein oligonucleotide barcodes associated with different solid supports comprise different cell label sequences,
wherein oligonucleotide barcodes situated within the same chamber comprise the same chamber indexing subsequence(s), and wherein oligonucleotide barcodes situated within different chambers comprise different chamber indexing subsequence(s);
partitioning each of two or more populations of single cells to the partitions of an identified chamber of the plurality of chambers,
wherein each of the two or more populations of single cells comprises a plurality of single cells, wherein the single cells comprise copies of a nucleic acid target,
wherein single cells of the same population are situated within the same chamber, and wherein single cells of different populations are situated within different chambers;
barcoding the copies of a nucleic acid target from at least one of the plurality of single cells of at least one population of single cells using the plurality of oligonucleotide barcodes to generate a plurality of barcoded nucleic acid targets;
obtaining sequencing data comprising a plurality of sequencing reads of the plurality of barcoded nucleic acid targets, or products thereof;
identifying the chamber indexing subsequence(s) of each cell label sequence in the sequencing data; and
assigning each of the plurality of sequencing reads to a chamber of the plurality of chambers based on the chamber indexing subsequence(s) of each cell label sequence in the sequencing data.

3. A method of assigning sequencing data to a population of single cells, comprising:
providing a microwell array comprising a plurality of chambers, wherein each chamber comprises a plurality of partitions;
partitioning each of two or more pluralities of solid supports to the partitions of an identified chamber of the plurality of chambers, wherein the solid supports each comprise a plurality of oligonucleotide barcodes each comprising a cell label sequence, wherein each cell label sequence comprises predetermined chamber indexing subsequence(s),
wherein oligonucleotide barcodes associated with the same solid support comprise the same cell label sequence, wherein oligonucleotide barcodes associated with different solid supports comprise different cell label sequences,
wherein oligonucleotide barcodes situated within the same chamber comprise the same chamber indexing subsequence(s), and wherein oligonucleotide barcodes situated within different chambers comprise different chamber indexing subsequence(s);
partitioning each of two or more populations of single cells to the partitions of an identified chamber of the plurality of chambers,
wherein each of the two or more populations of single cells comprises a plurality of single cells, wherein the single cells comprise copies of a nucleic acid target,
wherein single cells of the same population are situated within the same chamber, and wherein single cells of different populations are situated within different chambers;
barcoding the copies of a nucleic acid target from at least one of the plurality of single cells of at least one population of single cells using the plurality of oligonucleotide barcodes to generate a plurality of barcoded nucleic acid targets;
obtaining sequencing data comprising a plurality of sequencing reads of the plurality of barcoded nucleic acid targets, or products thereof;
identifying the chamber indexing subsequence(s) of each cell label sequence in the sequencing data;
assigning each of the plurality of sequencing reads to a chamber of the plurality of chambers based on the chamber indexing subsequence(s) of each cell label sequence in the sequencing data; and
assigning each of the plurality of sequencing reads to a population of the two or more populations of single cells based on the chamber assigned to the sequencing read.

4. A method of assigning sequencing data to a population of single cells, comprising:
providing a microwell array comprising a plurality of chambers, wherein each chamber comprises a plurality of partitions, wherein each chamber comprises a predetermined plurality of solid supports selected from two or more pluralities of solid supports, wherein the solid supports are situated within the partitions of said chambers, wherein the solid supports each comprise a plurality of oligonucleotide barcodes each comprising a cell label sequence, wherein each cell label sequence comprises predetermined chamber indexing subsequence(s),
wherein oligonucleotide barcodes associated with the same solid support comprise the same cell label sequence, wherein oligonucleotide barcodes associated with different solid supports comprise different cell label sequences,
wherein oligonucleotide barcodes situated within the same chamber comprise the same chamber indexing subsequence(s), and wherein oligonucleotide barcodes situated within different chambers comprise different chamber indexing subsequence(s);
partitioning each of two or more populations of single cells to the partitions of an identified chamber of the plurality of chambers,
wherein each of the two or more populations of single cells comprises a plurality of single cells, wherein the single cells comprise copies of a nucleic acid target,
wherein single cells of the same population are situated within the same chamber, and wherein single cells of different populations are situated within different chambers;
barcoding the copies of a nucleic acid target from at least one of the plurality of single cells of at least one population of single cells using the plurality of oligonucleotide barcodes to generate a plurality of barcoded nucleic acid targets;
obtaining sequencing data comprising a plurality of sequencing reads of the plurality of barcoded nucleic acid targets, or products thereof;
identifying the chamber indexing subsequence(s) of each cell label sequence in the sequencing data; and
assigning each of the plurality of sequencing reads to a chamber of the plurality of chambers based on the chamber indexing subsequence(s) of each cell label sequence in the sequencing data; and
assigning each of the plurality of sequencing reads to a population of the two or more populations of single cells based on the chamber assigned to the sequencing read.

5. A method of associating sequencing data and phenotypic data of a population of obtaining phenotypic data of each of two or more populations of single cells;
providing a microwell array comprising a plurality of chambers, wherein each chamber comprises a plurality of partitions;
partitioning each of two or more pluralities of solid supports to the partitions of an identified chamber of the plurality of chambers, wherein the solid supports each comprise a plurality of oligonucleotide barcodes each comprising a cell label sequence, wherein each cell label sequence comprises predetermined chamber indexing subsequence(s),
wherein oligonucleotide barcodes associated with the same solid support comprise the same cell label sequence, wherein oligonucleotide barcodes associated with different solid supports comprise different cell label sequences,
wherein oligonucleotide barcodes situated within the same chamber comprise the same chamber indexing subsequence(s), and wherein oligonucleotide barcodes situated within different chambers comprise different chamber indexing subsequence(s);
partitioning each of two or more populations of single cells to the partitions of an identified chamber of the plurality of chambers,
wherein each of the two or more populations of single cells comprises a plurality of single cells, wherein the single cells comprise copies of a nucleic acid target,
wherein single cells of the same population are situated within the same chamber, and wherein single cells of different populations are situated within different chambers;
barcoding the copies of a nucleic acid target from at least one of the plurality of single cells of at least one population of single cells using the plurality of oligonucleotide barcodes to generate a plurality of barcoded nucleic acid targets;
obtaining sequencing data comprising a plurality of sequencing reads of the plurality of barcoded nucleic acid targets, or products thereof;
identifying the chamber indexing subsequence(s) of each cell label sequence in the sequencing data; and
associating the sequencing data and the phenotypic data of at least one population of single cells based on the chamber indexing subsequence(s) of each cell label sequence in the sequencing data.

6. A method of associating sequencing data and phenotypic data of a population of single cells, comprising:
obtaining phenotypic data of each of two or more populations of single cells;
providing a microwell array comprising a plurality of chambers, wherein each chamber comprises a plurality of partitions, wherein each chamber comprises a predetermined plurality of solid supports selected from two or more pluralities of solid supports, wherein the solid supports are situated within the partitions of said chambers, wherein the solid supports each comprise a plurality of oligonucleotide barcodes each comprising a cell label sequence, wherein each cell label sequence comprises predetermined chamber indexing subsequence(s),
wherein oligonucleotide barcodes associated with the same solid support comprise the same cell label sequence, wherein oligonucleotide barcodes associated with different solid supports comprise different cell label sequences,
wherein oligonucleotide barcodes situated within the same chamber comprise the same chamber indexing subsequence(s), and wherein oligonucleotide barcodes situated within different chambers comprise different chamber indexing subsequence(s);
partitioning each of two or more populations of single cells to the partitions of an identified chamber of the plurality of chambers,
wherein each of the two or more populations of single cells comprises a plurality of single cells, wherein the single cells comprise copies of a nucleic acid target,
wherein single cells of the same population are situated within the same chamber, and wherein single cells of different populations are situated within different chambers;
barcoding the copies of a nucleic acid target from at least one of the plurality of single cells of at least one population of single cells using the plurality of oligonucleotide barcodes to generate a plurality of barcoded nucleic acid targets;
obtaining sequencing data comprising a plurality of sequencing reads of the plurality of barcoded nucleic acid targets, or products thereof;
identifying the chamber indexing subsequence(s) of each cell label sequence in the sequencing data; and
associating the sequencing data and the phenotypic data of at least one population of single cells based on the chamber indexing subsequence(s) of each cell label sequence in the sequencing data.

7. The method of any one of claims 1-6, wherein the two or more pluralities of solid supports comprise a plurality of first solid supports and a plurality of second solid supports,
wherein oligonucleotide barcodes associated with plurality of first solid supports have a first predetermined chamber indexing subsequence(s),
wherein oligonucleotide barcodes associated with plurality of second solid supports have a second predetermined chamber indexing subsequence(s), and
wherein the first predetermined chamber indexing subsequence(s) and the second predetermined chamber indexing subsequence(s) are different.

8. The composition of any one of claims 1-7, wherein the two or more pluralities of solid supports comprise a plurality of first solid supports and a plurality of second solid supports,
wherein the first predetermined chamber indexing subsequence(s) of the plurality of first solid supports are selected from a first set of chamber indexing subsequence(s),
wherein the second predetermined chamber indexing subsequence(s) of the plurality of second solid supports are selected from a second set of chamber indexing subsequence(s), and
wherein each of the chamber indexing subsequence(s) of first set of chamber indexing subsequence(s) differ from the chamber indexing subsequence(s) of the second set of chamber indexing subsequence(s).

9. The composition of any one of claims 1-8, wherein, based on the predetermined chamber indexing subsequence(s) of a sequencing read derived from an oligonucleotide barcode, or a product thereof, a user can determine if the oligonucleotide barcode is associated with the plurality of first solid supports or the plurality of second solid supports.

10. The method of any one of claim 1-2 or 5-6, comprising:
assigning each of the plurality of sequencing reads to a population of the two or more populations of single cells based on the chamber assigned to the sequencing read.

11. The method of any one of claim 1-4 or 10, comprising:
obtaining phenotypic data of each of two or more populations of single cells; and
associating the sequencing data and the phenotypic data of at least one population of single cells based on the chamber indexing subsequence(s) of each cell label sequence in the sequencing data.

12. The method of any one of claims 1-11, wherein partitioning each of two or more pluralities of solid supports to the partitions of an identified chamber of the plurality of chambers comprises partitioning a predetermined plurality of solid supports selected from two or more pluralities of solid supports to an identified chamber of the plurality of chambers.

13. The method of any one of claims 1-12, wherein barcoding the copies of a nucleic acid target comprises:
contacting the plurality of oligonucleotide barcodes with the copies of a nucleic acid target for hybridization; and
extending the plurality of oligonucleotide barcodes hybridized to the copies of a nucleic acid target to generate a plurality of barcoded nucleic acid targets.

14. The method of any one of claims 1-13, wherein a partition is a microwell having a volume ranging from about 1,000 μm³ to about 786,000 μm³.

15. The method of any one of claims 1-14, wherein the oligonucleotide barcodes each comprise a molecular label sequence.

16. The method of any one of claims 1-15, wherein oligonucleotide barcodes of the same plurality of solid supports comprise the same chamber indexing subsequence(s), and wherein oligonucleotide barcodes of different pluralities of solid supports comprise different chamber indexing subsequence(s).

17. The method of any one of claims 1-16, wherein:
each cell label of the plurality of oligonucleotide barcodes comprises at least 6 nucleotides;
the cell label comprises a plurality of portions and one or more linkers;
the cell label comprises a first cell label portion, a first linker, and a second cell label portion, optionally the cell label comprises a second linker and a third cell label portion, further optionally the cell label comprises a third linker and a fourth cell label portion; and/or
the first cell label portion, the second cell label portion, the third cell label portion, and/or the fourth cell label portion comprises the chamber indexing subsequence(s); and/or
chamber indexing subsequence(s) is 2-80 nucleotides in length.

18. The method of any one of claims 1-17, wherein the populations of single cells are distinct samples.

19. The method of any one of claims 1-18, wherein each population of the two or more populations of single cells is a biological replicate sample, a technical replicate sample, a control sample, an experimental sample, or a combination thereof.

20. The method of any one of claims 1-19, wherein the two or more populations of single cells are derived from one or more samples separated on the basis of phenotypic data, optionally the plurality of single cells comprises T cells, B cells, tumor cells, myeloid cells, blood cells, normal cells, fetal cells, maternal cells, or a mixture thereof.

21. The method of any one of claims 1-20, wherein the plurality of chambers is at least about 4 chambers, about 10 chambers, about 20 chambers, about 40 chambers, about 60 chambers, about 80 chambers, about 100 chambers, about 200 chambers, about 300 chambers, about 400 chambers, about 500 chambers, about 750 chambers, or about 1000 chambers, optionally the plurality of chambers comprises 8 lanes.

22. The method of any one of claims 1-21, wherein the plurality of partitions is at least about 100 partitions, about 500 partitions, about 1000 partitions, about 5000 partitions, about 10000 partitions, about 25000 partitions, about 50000 partitions, about 75000 partitions, or about 100000 partitions.

23. The method of any one of claims 1-22, wherein the sequencing of the cell label identifies the chamber of origin of each sequenced barcoded nucleic acid target, or products thereof, within the microwell array.

24. The method of any one of claims 1-23, wherein a partition of the plurality of partitions comprises a single cell of the plurality of single cells and a single solid support of the plurality of solid supports.

25. The method of any one of claims 1-24, wherein the plurality of barcoded nucleic acid targets each comprise a sequence complementary to at least a portion of the nucleic acid target and the molecular label.

26. The method of any one of claims 1-25, wherein each of the plurality of sequencing reads comprise (1) a cell label sequence and (2) a molecular label sequence.

27. The method of any one of claims 1-26, wherein each oligonucleotide barcode comprises a first universal sequence.

28. The method of any one of claims 1-27, wherein the oligonucleotide barcode comprises a target-binding region comprising a capture sequence.

29. The method of claim 28, wherein the target-binding region comprises a gene-specific sequence, an oligo(dT) sequence, a random multimer, or any combination thereof.

30. The method of any one of claims 1-29, wherein partitioning a plurality of solid supports to the partitions of each of the plurality of chambers comprises flow cytometrically depositing each plurality of solid supports into an identified chamber of the microwell array.

31. The method of any one of claims 1-30, wherein partitioning each of two or more populations of single cells to the partitions of an identified chamber of the plurality of chambers comprises flow cytometrically depositing each population of single cells into an identified chamber of the microwell array.

32. The method of any one of claims 1-31, further comprising aligning a sorting component of a flow cytometer with the microwell array.

33. The method of any one of claims 1-32, wherein the phenotypic data comprises event data.

34. The method of claim 33, wherein the event data comprises quantitative biological event data derived from a sorting device.

35. The method of any one of claims 33-34, wherein the event data comprises a side scatter signal, a forward scatter signal, one or more fluorescence signals, or any combination thereof.

36. The method of any one of claims 1-35, comprising correlation analysis of the phenotypic data and the sequencing data of the single cells.

37. The method of claim 36, wherein the correlation analysis identifies one or more of the following: candidate biomarkers, candidate therapeutic agents, candidate doses of therapeutic agents, and/or cellular targets of candidate therapeutic agents.

38. The method of any one of claims 1-37, comprising lysing the one or more of the single cells.

39. The method of any one of claims 1-38, wherein the viability of the single cells is maintained for a period of time after partitioning and before lysis, optionally the period of time is at least about 10 min, 20 min, 30 min, 40 min, 50 min, 60 min, 100 min, 250 min, 500 min, 750 min, 1000 min, 2500 min, 5000 min, 7500 min, or 10000 min.

40. The method of any one of claims 1-39, further comprising determining the copy number of the nucleic acid target in one or more of the plurality of single cells of at least one population of single cells.

41. The method of claim 40, wherein determining the copy number of the nucleic acid target in one or more of the plurality of single cells comprises determining the copy number of the nucleic acid target in the plurality of single cells based on the number of molecular labels with distinct sequences, complements thereof, or a combination thereof, associated with the plurality of barcoded nucleic acid targets, or products thereof.

42. The method of any one of claims 40-41, comprising:
contacting random primers with the plurality of barcoded nucleic acid targets, wherein each of the random primers comprises a second universal sequence, or a complement thereof; and
extending the random primers hybridized to the plurality of barcoded nucleic acid targets to generate a plurality of extension products.

43. The method of claim 42, comprising amplifying the plurality of extension products using primers capable of hybridizing to the first universal sequence or complements thereof, and primers capable of hybridizing the second universal sequence or complements thereof, thereby generating a first plurality of barcoded amplicons.

44. The method of claim 43, wherein amplifying the plurality of extension products comprises adding sequences of binding sites of sequencing primers and/or sequencing adaptors, complementary sequences thereof, and/or portions thereof, to the plurality of extension products.

45. The method of any one of claims 43-44, comprising determining the copy number of the nucleic acid target in one or more of the plurality of single cells based on the number of molecular labels with distinct sequences associated with the first plurality of barcoded amplicons, or products thereof.

46. The method of any one of claims 43-45, wherein determining the copy number of the nucleic acid target in one or more of the plurality of single cells comprises determining the number of each of the plurality of nucleic acid targets in one or more of the plurality of single cells based on the number of the molecular labels with distinct sequences associated with barcoded amplicons of the first plurality of barcoded amplicons comprising a sequence of the each of the plurality of nucleic acid targets.

47. The method of claim 46, wherein the sequence of the each of the plurality of nucleic acid targets comprises a subsequence of the each of the plurality of nucleic acid targets.

48. The method of any one of claims 43-47, wherein the sequence of the nucleic acid target in the first plurality of barcoded amplicons comprises a subsequence of the nucleic acid target.

49. The method of any one of claims 43-48, comprising amplifying the first plurality of barcoded amplicons using primers capable of hybridizing to the first universal sequence or complements thereof, and primers capable of hybridizing the second universal sequence or complements thereof, thereby generating a second plurality of barcoded amplicons.

50. The method of claim 49, wherein amplifying the first plurality of barcoded amplicons comprises adding sequences of binding sites of sequencing primers and/or sequencing adaptors, complementary sequences thereof, and/or portions thereof, to the first plurality of barcoded amplicons.

51. The method of any one of claims 49-50, comprising determining the copy number of the nucleic acid target in one or more of the plurality of single cells based on the number of molecular labels with distinct sequences associated with the second plurality of barcoded amplicons, or products thereof.

52. The method of any one of claims 43-51, wherein the first plurality of barcoded amplicons and/or the second plurality of barcoded amplicons comprise whole transcriptome amplification (WTA) products.

53. The method of any one of claims 1-52, comprising synthesizing a third plurality of barcoded amplicons using the plurality of barcoded nucleic acid targets as templates to generate a third plurality of barcoded amplicons, optionally:
wherein synthesizing a third plurality of barcoded amplicons comprises performing polymerase chain reaction (PCR) amplification of the plurality of the barcoded nucleic acid targets;
wherein synthesizing a third plurality of barcoded amplicons comprises PCR amplification using primers capable of hybridizing to the first universal sequence, or a complement thereof, and a target-specific primer;
comprising obtaining sequence data of the third plurality of barcoded amplicons, or products thereof, and optionally obtaining the sequence information comprises attaching sequencing adaptors to the third plurality of barcoded amplicons, or products thereof; and/or
comprising determining the copy number of the nucleic acid target in one or more of the plurality of single cells based on the number of molecular labels with distinct sequences associated with the third plurality of barcoded amplicons, or products thereof.

54. The method of any one of claims 1-53, wherein:
the nucleic acid target comprises a nucleic acid molecule, optionally the nucleic acid molecule comprises ribonucleic acid (RNA), messenger RNA (mRNA), microRNA, small interfering RNA (siRNA), RNA degradation product, RNA comprising a poly(A) tail, or any combination thereof;
the nucleic acid target comprises a sample indexing oligonucleotide, and optionally the sample indexing oligonucleotide comprises a sample indexing sequence, and sample indexing sequences of at least two sample indexing compositions of a plurality of sample indexing compositions comprise different sequences; and/or
the nucleic acid target comprises a cellular component-binding reagent specific oligonucleotide, optionally a cellular component-binding reagent specific oligonucleotide comprises a unique identifier sequence for a cellular component-binding reagent.

55. The method of any one of claims 1-54, wherein:
extending the plurality of oligonucleotide barcodes comprises extending the plurality of oligonucleotide barcodes using a reverse transcriptase and/or a DNA polymerase lacking at least one of 5′ to 3′ exonuclease activity and 3′ to 5′ exonuclease activity;
the DNA polymerase comprises a Klenow Fragment;
the reverse transcriptase comprises a viral reverse transcriptase, optionally wherein the viral reverse transcriptase is a murine leukemia virus (MLV) reverse transcriptase or a Moloney murine leukemia virus (MMLV) reverse transcriptase;
the first universal sequence and the second universal sequence are the same;
the first universal sequence and the second universal sequence are different;
the first universal sequence, and/or the second universal sequence comprise the binding sites of sequencing primers and/or sequencing adaptors, complementary sequences thereof, and/or portions thereof;
the sequencing adaptors comprise a P5 sequence, a P7 sequence, complementary sequences thereof, and/or portions thereof;
the sequencing primers comprise a Read 1 sequencing primer, a Read 2 sequencing primer, complementary sequences thereof, and/or portions thereof;
at least 10 of the plurality of oligonucleotide barcodes comprise different molecular label sequences; and/or
each molecular label of the plurality of oligonucleotide barcodes comprises at least 6 nucleotides.

56. The method of any one of claims 1-55, wherein the solid support comprises a planar surface or a synthetic particle, optionally wherein:
at least one of the plurality of oligonucleotide barcodes is immobilized on, partially immobilized, enclosed in, or partially enclosed in the synthetic particle;
the synthetic particle is disruptable;
the synthetic particle comprises a bead;
the bead comprises a Sepharose bead, a streptavidin bead, an agarose bead, a magnetic bead, a conjugated bead, a protein A conjugated bead, a protein G conjugated bead, a protein A/G conjugated bead, a protein L conjugated bead, an oligo(dT) conjugated bead, a silica bead, a silica-like bead, an anti-biotin microbead, an anti-fluorochrome microbead, or any combination thereof;
the synthetic particle comprises a material selected from the group consisting of polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, and any combination thereof; and/or
the synthetic particle comprises a disruptable hydrogel particle.

57. A composition, comprising:
two or more pluralities of solid supports,
wherein the solid supports each comprise a plurality of oligonucleotide barcodes each comprising a cell label sequence, wherein each cell label sequence comprises predetermined chamber indexing subsequence(s), and
wherein oligonucleotide barcodes associated with the same solid support comprise the same cell label sequence, wherein oligonucleotide barcodes associated with different solid supports comprise different cell label sequences.

58. The composition of claim 57, wherein the two or more pluralities of solid supports comprise a plurality of first solid supports and a plurality of second solid supports,
wherein oligonucleotide barcodes associated with plurality of first solid supports have a first predetermined chamber indexing subsequence(s),
wherein oligonucleotide barcodes associated with plurality of second solid supports have a second predetermined chamber indexing subsequence(s), and
wherein the first predetermined chamber indexing subsequence(s) and the second predetermined chamber indexing subsequence(s) are different.

59. The composition of any one of claims 57-58, wherein the two or more pluralities of solid supports comprise a plurality of first solid supports and a plurality of second solid supports,
wherein the first predetermined chamber indexing subsequence(s) of the plurality of first solid supports are selected from a first set of chamber indexing subsequence(s),
wherein the second predetermined chamber indexing subsequence(s) of the plurality of second solid supports are selected from a second set of chamber indexing subsequence(s), and
wherein each of the chamber indexing subsequence(s) of first set of chamber indexing subsequence(s) differ from the chamber indexing subsequence(s) of the second set of chamber indexing subsequence(s).

60. The composition of any one of claims 57-59, wherein, based on the predetermined chamber indexing subsequence(s) of a sequencing read derived from an oligonucleotide barcode, or a product thereof, a user can determine if the oligonucleotide barcode is associated with the plurality of first solid supports or the plurality of second solid supports.

61. The composition of any one of claims 57-60, wherein the two or more pluralities of solid supports comprise:
a plurality of third solid supports, optionally: (a) oligonucleotide barcodes associated with the plurality of third solid supports have a third chamber indexing subsequence(s) and/or (b) third chamber indexing subsequence(s) of the plurality of third solid supports are selected from a third set of chamber indexing subsequence(s);
a plurality of fourth solid supports, optionally: (a) oligonucleotide barcodes associated with the plurality of fourth solid supports have a fourth chamber indexing subsequence(s) and/or (b) fourth chamber indexing subsequence(s) of the plurality of fourth solid supports are selected from a fourth set of chamber indexing subsequence(s);
a plurality of fifth solid supports, optionally: (a) oligonucleotide barcodes associated with the plurality of fifth solid supports have a fifth chamber indexing subsequence(s) and/or (b) fifth chamber indexing subsequence(s) of the plurality of fifth solid supports are selected from a fifth set of chamber indexing subsequence(s);
a plurality of sixth solid supports, optionally: (a) oligonucleotide barcodes associated with the plurality of sixth solid supports have a sixth chamber indexing subsequence(s) and/or (b) sixth chamber indexing subsequence(s) of the plurality of sixth solid supports are selected from a sixth set of chamber indexing subsequence(s);
a plurality of seventh solid supports, optionally: (a) oligonucleotide barcodes associated with the plurality of seventh solid supports have a seventh chamber indexing subsequence(s) and/or (b) seventh chamber indexing subsequence(s) of the plurality of seventh solid supports are selected from a seventh set of chamber indexing subsequence(s); and/or
a plurality of eighth solid supports, optionally: (a) oligonucleotide barcodes associated with the plurality of eighth solid supports have a eighth chamber indexing subsequence(s) and/or (b) eighth chamber indexing subsequence(s) of the plurality of eighth solid supports are selected from an eighth set of chamber indexing subsequence(s), and
wherein the first chamber indexing subsequence(s), the second chamber indexing subsequence(s), the third chamber indexing subsequence(s), the fourth chamber indexing subsequence(s), the fifth chamber indexing subsequence(s), the sixth chamber indexing subsequence(s), the seventh chamber indexing subsequence(s), and/or the eighth chamber indexing subsequence(s), do not share a sequence with each other.

62. The composition of any one of claims 57-61, wherein, based on the predetermined chamber indexing subsequence(s) of a sequencing read derived from an oligonucleotide barcode, or a product thereof, a user can determine if the oligonucleotide barcode is associated with a first solid support, a second solid support, a third solid support, a fourth solid support, a fifth solid support, a sixth solid support, a seventh solid support, or an eighth solid support.

63. The composition of any one of claims 57-62, wherein the cell label comprises a plurality of cell label portions and one or more linkers.

64. The composition of any one of claims 57-63, wherein the cell label comprises a first cell label portion, a first linker, and a second cell label portion, optionally the cell label comprises a second linker and a third cell label portion, further optionally the cell label comprises a third linker and a fourth cell label portion.

65. The composition of any one of claims 57-64, wherein the predetermined chamber indexing subsequence(s) comprise the first cell label portion, the second cell label portion, the third cell label portion, the fourth cell label portion, or any combination thereof.

66. The composition of any one of claims 57-65, wherein the first, second, third, fourth, fifth, sixth, seventh, and/or eighth set of chamber indexing subsequence(s) comprises a set of less than about 960, about 864, about 768, about 672, about 576, about 480, about 384, about 288, about 192, about 96, or about 48, unique sequences distinct from the chamber indexing subsequence(s) of other sets of chamber indexing subsequence(s).

67. The composition of any one of claims 57-66, wherein the first cell label portion, the second cell label portion, the third cell label portion, the fourth cell label portion, or any combination thereof, are selected from a set of less than about 480, about 384, about 288, about 192, about 96, or about 48, unique sequences.

68. The composition of any one of claims 57-67, wherein, based on the sequence(s) of the first cell label portion, the second cell label portion, the third cell label portion, the fourth cell label portion, or any combination thereof, of a sequencing read derived from an oligonucleotide barcode, or a product thereof, a user can determine if the oligonucleotide barcode is associated with a first solid support, a second solid support, a third solid support, a fourth solid support, a fifth solid support, a sixth solid support, a seventh solid support, or an eighth solid support.

69. A method, comprising:
distributing a plurality of precursor first solid supports and a first population of first oligonucleotides to a first plurality of first partitions, wherein colocalized solid supports and first oligonucleotides become associated;
distributing a plurality of precursor second solid supports and a second population of first oligonucleotides to a first plurality of second partitions, wherein colocalized solid supports and first oligonucleotides become associated;
pooling the plurality of precursor first solid supports associated with first oligonucleotides;
pooling the plurality of precursor second solid supports associated with first oligonucleotides;
distributing the plurality of precursor first solid supports associated with first oligonucleotides and a first population of second oligonucleotides to a second plurality of first partitions, wherein colocalized solid supports and second oligonucleotides become associated;
distributing the plurality of precursor second solid supports associated with first oligonucleotides and a second population of second oligonucleotides to a second plurality of second partitions, wherein colocalized solid supports and second oligonucleotides become associated;
pooling the plurality of precursor first solid supports associated with first and second oligonucleotides;
pooling the plurality of precursor second solid supports associated with first and second oligonucleotides;
distributing the plurality of precursor first solid supports associated with first and second oligonucleotides and a first population of third oligonucleotides to a third plurality of first partitions, wherein colocalized solid supports and third oligonucleotides become associated; and
distributing the plurality of precursor second solid supports associated with first and second oligonucleotides and a second population of third oligonucleotides to a third plurality of second partitions, wherein colocalized solid supports and third oligonucleotides become associated.

70. The method of claim 69, comprising:
pooling the plurality of precursor first solid supports associated with first, second, and third oligonucleotides to generate a plurality of first solid supports; and
pooling the plurality of precursor second solid supports associated with first, second, and third oligonucleotides to generate a plurality of second solid supports.

71. The method of any one of claims 69-70, wherein:
(i) the first and second populations of first oligonucleotides are the same, the first and second populations of second oligonucleotides are the same, and the first and second populations of third oligonucleotides are different;
(ii) the first and second populations of first oligonucleotides are the same, the first and second populations of second oligonucleotides are different, and the first and second populations of third oligonucleotides are the same;
(iii) the first and second populations of first oligonucleotides are the same, the first and second populations of second oligonucleotides are different, and the first and second populations of third oligonucleotides are different;
(iv) the first and second populations of first oligonucleotides are different, the first and second populations of second oligonucleotides are different, and the first and second populations of third oligonucleotides are different;
(v) the first and second populations of first oligonucleotides are different, the first and second populations of second oligonucleotides are different, and the first and second populations of third oligonucleotides are the same;
(vi) the first and second populations of first oligonucleotides are different, the first and second populations of second oligonucleotides are the same, and the first and second populations of third oligonucleotides are different; or
(vii) the first and second populations of first oligonucleotides are different, the first and second populations of second oligonucleotides are the same, and the first and second populations of third oligonucleotides are the same.

72. The method of any one of claims 69-71, wherein:
wherein the first and second populations of first oligonucleotides each comprise about 192 first cell label portions with distinct sequences;
wherein the first and second populations of second oligonucleotides each comprise about 192 second cell label portions with distinct sequences; and
wherein the first and second populations of third oligonucleotides each comprise about 192 third cell label portions with distinct sequences.

73. The method of any one of claims 69-72, wherein:
the first and second populations of first oligonucleotides are the same;
the first and second populations of second oligonucleotides are the same; and
the first and second populations of third oligonucleotides are different,
optionally the first and second populations of third oligonucleotides comprise non-overlapping subsets of the cell label portion sequences of the first and second populations of first oligonucleotides and/or the first and second populations of second oligonucleotides.

74. The method of any one of claims 69-73, wherein:
wherein the first and second populations of first oligonucleotides each comprise about 384 first cell label portions with distinct sequences;
wherein the first and second populations of second oligonucleotides each comprise about 384 second cell label portions with distinct sequences; and
wherein the first and second populations of third oligonucleotides each comprise about 48 third cell label portions with distinct sequences.

75. The method of any one of claims 69-74, comprising:
distributing the plurality of precursor first solid supports associated with first, second, and third oligonucleotides and fourth oligonucleotides to a fourth plurality of first partitions, wherein colocalized solid supports and fourth oligonucleotides become associated;
distributing the plurality of precursor second solid supports associated with first, second, and third oligonucleotides and a fifth oligonucleotides to a fourth plurality of second partitions, wherein colocalized solid supports and fifth oligonucleotides become associated;
pooling the plurality of precursor first solid supports associated with first, second, third, and fourth oligonucleotides to generate a plurality of first solid supports; and
pooling the plurality of precursor second solid supports associated with first, second, third, and fifth oligonucleotides to generate a plurality of second solid supports.

76. The method of any one of claims 69-75, wherein:
the first and second populations of first oligonucleotides are the same;
the first and second populations of second oligonucleotides are the same;
the first and second populations of third oligonucleotides are the same; and
the fifth oligonucleotides and sixth oligonucleotides are different.

77. The method of any one of claims 69-76, wherein:
the first oligonucleotide comprises a first cell label portion and a first linker, or complements thereof;
the second oligonucleotide comprises a first linker, a second cell label portion, and a second linker, or complements thereof;
the third oligonucleotide comprises a second linker and a third cell label portion, or complements thereof, optionally the second oligonucleotide further comprises a third linker or complement thereof; and/or
the fourth oligonucleotide comprises a third linker and a fourth cell label portion, or complements thereof.

78. The method of any one of claims 69-77, wherein:
the first oligonucleotide and the second oligonucleotide are configured to connect via the first linker;
the second oligonucleotide and the third oligonucleotide are configured to connect via the second linker; and/or
the third oligonucleotide and the fourth oligonucleotide are configured to connect via the third linker.

79. The method of any one of claims 69-78, wherein the plurality of first solid supports and/or the plurality of second solid supports comprise at least about 1000, about 10000, about 100000, about 1000000, about 7000000, about 10000000, about 56000000, unique cell label sequences.

80. The method of any one of claims 69-79, wherein the sequence of the first second cell label portion, the second cell label portion, and/or the third cell label portion is the same.

81. The method of any one of claims 69-80, wherein the sequence of the first second cell label portion, the second cell label portion, and/or the third cell label portion is different.

82. The method of any one of claims 69-81, wherein the first oligonucleotide, the second oligonucleotide, the third oligonucleotide, and/or the fourth oligonucleotide are single-stranded, double-stranded, and/or comprise one or two single-stranded overhangs.

83. The method of any one of claims 69-82, wherein the first solid supports and second solid supports each comprise a plurality of oligonucleotide barcodes each comprising a cell label sequence, wherein each cell label sequence comprises predetermined chamber indexing subsequence(s).

84. The method of any one of claims 69-83,
wherein oligonucleotide barcodes associated with plurality of first solid supports have a first predetermined chamber indexing subsequence(s),
wherein oligonucleotide barcodes associated with plurality of second solid supports have a second predetermined chamber indexing subsequence(s), and
wherein the first predetermined chamber indexing subsequence(s) and the second predetermined chamber indexing subsequence(s) are different.

85. The method of any one of claims 69-84,
wherein the first predetermined chamber indexing subsequence(s) of the plurality of first solid supports are selected from a first set of chamber indexing subsequence(s),
wherein the second predetermined chamber indexing subsequence(s) of the plurality of second solid supports are selected from a second set of chamber indexing subsequence(s), and
wherein each of the chamber indexing subsequence(s) of first set of chamber indexing subsequence(s) differ from the chamber indexing subsequence(s) of the second set of chamber indexing subsequence(s).

86. The method of any one of claims 69-85, wherein the method generates the composition of any one of claims 57-68.

87. The method of any one of claims 69-86, further comprising:
providing a microwell array comprising a plurality of chambers, wherein each chamber comprises a plurality of partitions; and
partitioning the plurality of first solid supports and the plurality of second solid supports to the partitions of an identified first chamber and identified second chamber of the plurality of chambers, respectively.

88. The method of any one of claims 69-87, wherein distributing oligonucleotides to a plurality of partitions comprises providing a plurality of partitions comprising said oligonucleotides.

89. The method of any one of claims 69-88, wherein the plurality of partitions comprise a 384-well plate, a 288-well plate, a 192-well plate, a 96-well plate or a 48-well plate.

90. The method or composition of any one of claims 57-89, wherein the solid support comprises a synthetic particle, optionally wherein:
at least one oligonucleotide barcode of the plurality of oligonucleotide barcodes is immobilized on the synthetic particle, partially immobilized on the synthetic particle, enclosed in the synthetic particle, partially enclosed in the synthetic particle, or a combination thereof;
the synthetic particle is disruptable;
the synthetic particle comprises a bead, optionally the bead comprises a Sepharose bead, a streptavidin bead, an agarose bead, a magnetic bead, a conjugated bead, a protein A conjugated bead, a protein G conjugated bead, a protein A/G conjugated bead, a protein L conjugated bead, an oligo(dT) conjugated bead, a silica bead, a silica-like bead, an anti-biotin microbead, an anti-fluorochrome microbead, or any combination thereof;
the synthetic particle comprises a material selected from the group consisting of polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, and any combination thereof; and/or
the synthetic particle comprises a disruptable hydrogel particle.

91. The method of any one of claims 1-56, wherein the two or more pluralities of solid supports are the two or more pluralities of solid supports of any one of claims 57-68 and/or are generated by the method of any one of claims 69-89.

92. A composition comprising:
a microwell array,
wherein the microwell array comprises a plurality of chambers, wherein each chamber comprises a plurality of partitions, wherein each partition is a microwell having a volume ranging from about 1,000 μm³ to about 786,000 μm³, optionally:
further comprising a cartridge, wherein the cartridge comprises at least one of: an inlet port, an outlet port, a pump, a valve, a vent, a reservoir, a sample collection chamber, a temperature control apparatus, or any combination thereof.

93. A composition comprising:
a cartridge,
wherein the cartridge comprises at least one of: an inlet port, an outlet port, a pump, a valve, a vent, a reservoir, a sample collection chamber, a temperature control apparatus, or any combination thereof, wherein the cartridge comprises a microwell array, wherein the microwell array comprises a plurality of chambers, wherein each chamber comprises a plurality of partitions, wherein each partition is a microwell having a volume ranging from about 1,000 μm³ to about 786,000 μm³.

94. A composition comprising:
two or more pluralities of solid supports, wherein the solid supports each comprise a plurality of oligonucleotide barcodes each comprising a cell label sequence, wherein each cell label sequence comprises predetermined chamber indexing subsequence(s),
wherein oligonucleotide barcodes associated with the same solid support comprise the same cell label sequence, wherein oligonucleotide barcodes associated with different solid supports comprise different cell label sequences,
wherein oligonucleotide barcodes of the same plurality of solid supports comprise the same chamber indexing subsequence(s), and wherein oligonucleotide barcodes of different pluralities of solid supports comprise different chamber indexing subsequence(s).

95. The composition of any one of claims 92-94, wherein a partition of the plurality of partitions comprises a single solid support of a plurality of solid supports,
wherein the solid supports each comprise a plurality of oligonucleotide barcodes each comprising a cell label sequence, wherein each cell label sequence comprises predetermined chamber indexing subsequence(s),
wherein oligonucleotide barcodes associated with the same solid support comprise the same cell label sequence, wherein oligonucleotide barcodes associated with different solid supports comprise different cell label sequences,
wherein oligonucleotide barcodes situated within the same chamber comprise the same chamber indexing subsequence(s), and wherein oligonucleotide barcodes situated within different chambers comprise different chamber indexing subsequence(s).

96. The composition of any one of claims 92-95, wherein:
the cartridge is configured to maintain the viability of single cells partioned within said microwells, optionally the period of time is at least about 10 min, 20 min, 30 min, 40 min, 50 min, 60 min, 100 min, 250 min, 500 min, 750 min, 1000 min, 2500 min, 5000 min, 7500 min, or 10000 min;
the cartridge comprises a transparent window for optical imaging of the microwells;
the oligonucleotide barcodes each comprise a molecular label sequence;
each cell label of the plurality of oligonucleotide barcodes comprises at least 6 nucleotides;
the cell label comprises a plurality of portions and one or more linkers;
the cell label comprises a first cell label portion, a first linker, and a second cell label portion, optionally the cell label comprises a second linker and a third cell label portion, further optionally the cell label comprises a third linker and a fourth cell label portion;
the first cell label portion comprises the chamber indexing subsequence(s);
chamber indexing subsequence(s) is 2-15 nucleotides in length;
the plurality of chambers is at least about 4 chambers, about 10 chambers, about 20 chambers, about 40 chambers, about 60 chambers, about 80 chambers, about 100 chambers, about 200 chambers, about 300 chambers, about 400 chambers, about 500 chambers, about 750 chambers, or about 1000 chambers;
the plurality of partitions is at least about 100 partitions, about 500 partitions, about 1000 partitions, about 5000 partitions, about 10000 partitions, about 25000 partitions, about 50000 partitions, about 75000 partitions, or about 100000 partitions;
each oligonucleotide barcode comprises a first universal sequence;
the oligonucleotide barcode comprises a target-binding region comprising a capture sequence, optionally the target-binding region comprises a gene-specific sequence, an oligo(dT) sequence, a random multimer, or any combination thereof; and/or
each molecular label of the plurality of oligonucleotide barcodes comprises at least 6 nucleotides.

97. The composition of any one of claims 92-96, wherein the solid support comprises a planar surface or a synthetic particle, optionally wherein:
at least one oligonucleotide barcode of the plurality of oligonucleotide barcodes is immobilized on the synthetic particle, partially immobilized on the synthetic particle, enclosed in the synthetic particle, partially enclosed in the synthetic particle, or a combination thereof;
the synthetic particle is disruptable;
the synthetic particle comprises a bead, optionally the bead comprises a Sepharose bead, a streptavidin bead, an agarose bead, a magnetic bead, a conjugated bead, a protein A conjugated bead, a protein G conjugated bead, a protein A/G conjugated bead, a protein L conjugated bead, an oligo(dT) conjugated bead, a silica bead, a silica-like bead, an anti-biotin microbead, an anti-fluorochrome microbead, or any combination thereof;
the synthetic particle comprises a material selected from the group consisting of polydimethylsiloxane (PDMS), polystyrene, glass, polypropylene, agarose, gelatin, hydrogel, paramagnetic, ceramic, plastic, glass, methylstyrene, acrylic polymer, titanium, latex, Sepharose, cellulose, nylon, silicone, and any combination thereof; and/or
the synthetic particle comprises a disruptable hydrogel particle.

98. The composition of any one of claims 92-97, wherein the two or more pluralities of solid supports are the two or more pluralities of solid supports of any one of claims 57-68 and/or are generated by the method of any one of claims 69-89.

99. The composition of any one of claims 92-98, further comprising:
instructions for use;
a chamber indexing subsequence(s) lookup table, optionally wherein the chamber indexing subsequence(s) lookup table identifies the chamber indexing subsequence(s) associated with each solid support distributed in each microwell of the array;
an imaging system configured to capture and process images of all or a portion of the microwells, wherein the imaging system further comprises an illumination subsystem, an imaging subsystem, and a processor, optionally the imaging system is configured to perform bright-field, dark-field, fluorescence, or quantitative phase imaging;
a buffer; and/or
one or more reagents for a reverse transcription reaction, one or more reagents for an amplification reaction, or both.
